(19) United States
(12) Patent Application Publication
(10) Pub. No.: US 2018/0363035 A1
(43) Pub. Date: Dec. 20, 2018

(54) Methods and Compositions for Target Detection in a Nanopore Using a Labelled Polymer Scaffold

(71) Applicant: Two Pore Guys, Inc., Santa Cruz, CA (US)

(72) Inventors: Trevor J. Morin, Santa Cruz, CA (US); Daniel A. Heller, Santa Cruz, CA (US); William B. Dunbar, Santa Cruz, CA (US)

(21) Appl. No.: 15/573,630

(22) PCT Filed: May 16, 2016
(86) PCT No.: PCT/US16/32784

§ 371 Date: Nov. 13, 2017

Related U.S. Application Data
(60) Provisional application No. 62/162,619, filed on May 15, 2015.

(51) Int. Cl. C12Q 1/6818

(52) U.S. Cl. CPC C12Q 1/6818

(57) ABSTRACT

Provided are methods and compositions for detecting a target analyte suspected to be present in a sample with background molecules using a nanopore device. A plurality of probes for polymer scaffold identification or for target analyte binding and detection are provided.

### CROSS-REFERENCE TO RELATED APPLICATIONS

This application claims the benefit under 35 U.S.C § 119(e) to U.S. provisional application No. 62/162,619, filed May 15, 2015, the contents of which are incorporated by reference in their entirety.

### BACKGROUND

Methods and systems for highly sensitive detection of analytes, such as molecules, tumor cells, pathogenic organisms, have broad applications, in particular, clinically, for pathogen detection and disease diagnosis, for instance. Additionally, such detection can: allow for the personalization of medical treatments and health programs; facilitate the search for effective pharmaceutical drug compounds and biotherapeutics; and enable clinicians to identify abnormal hormones, ions, proteins, or other molecules produced by a patient's body and/or identify the presence of poisons, illegal drugs, or other harmful chemicals ingested or injected into a patient.

Nanopores have shown great promise as a low cost, low-energy, tiny sensor capable of detecting biological molecules for a range of purposes, from sequencing DNA to detecting target analytes that indicate the presence of diseases, pathogens, or other biomarkers of interest. A nanopore device can detect a molecule passing through a nanopore by a current impedance signal. The problem has been that the current impedance (or equivalent, e.g., current or voltage) information produced by a nanopore does not have sufficient resolution to distinguish the molecule. Several different molecules that pass through produce such similar electrical signals, so that it is nearly impossible to discriminate one from another.

There have been attempts to bind additional molecules to target analytes, so as to create larger current impedance (or equivalent) signals that can then be more easily identified, but this has shown to be insufficient when combined with original (even filtered) natural fluids (blood, saliva, urine, etc.), which have a vast population of background molecules that produce false positives, generating a high error rate of detection. Adding sophisticated sample preparation to filter out non-target markers technically helps, but the added cost and complexity exceeds that of existing non-nanopore technologies in use today. What is needed, therefore are methods and compositions for improved accuracy of detection of biological molecules and other analytes from a sample.

### SUMMARY

Various aspects disclosed herein can fulfill one or more of the above-mentioned needs. The systems and methods described herein each have several aspects, no single one of which is solely responsible for its desirable attributes. Without limiting the scope of this disclosure as expressed by the claims that follow, the more prominent features will now be discussed briefly. After considering this discussion, and particularly after reading the section entitled “Detailed Description,” one will understand how the sample features described herein provide for improved systems and methods.

In some embodiments, provided herein is a method of detecting a target analyte suspected to be present in a mixed sample, the method comprising: providing a nanopore device comprising a nanopore that separates an interior space of the device into a first volume and a second volume; loading a mixed sample suspected to contain a target analyte into the first volume of said nanopore device; loading a polymer scaffold into the first volume of said nanopore device; configuring the device to pass the polymer scaffold through the nanopore from the first volume to the second volume, wherein said polymer scaffold comprises a label or a detectable tag, and wherein said polymer scaffold comprises a target analyte binding site adapted to bind to said target analyte; recording an electrical signal generated by passage of said polymer scaffold through said nanopore from the first volume to the second volume; and analyzing said electrical signal to determine the presence or absence of a label and the presence or absence of a bound target analyte.

In some embodiments, the analysis comprises detecting a step transition within an event. In some embodiments, detecting a step transition event comprises identifying changes in said electrical signal wherein the finite difference exceeds a defined threshold. In some embodiments, the method further comprises detecting the presence of at least 2, 3, 4, 5, 6, or 7 levels in an electrical signal. In some embodiments, the method further comprises identifying the duration and amplitude of each of said levels. In some embodiments, the method further comprises assigning at least one of said levels to a physical status of the polymer scaffold. In some embodiments, the physical status of the polymer scaffold is selected from the group consisting of: unfolded, folded, label-bound, label-unbound, target analyte-unbound, and target analyte-bound. In some embodiments, the physical status is assigned using a binning scheme to correlate said level with said physical status.

In some embodiments, the analysis of the electrical signal obtained by the method of detecting a target analyte comprises linear filtering of the electronic signal. In some embodiments, the analysis of the electrical signal comprises fitting a multi-level approximation to said electrical signal. In some embodiments, the analysis of the electrical signal distinguishes detection of secondary structure of said polymer scaffold from said label bound to said polymer scaffold. In some embodiments, the analysis of the electrical signal is computer-implemented.

In some embodiments, the polymer scaffold is bound to a fusion molecule comprising said target analyte binding site. In some embodiments, the fusion molecule comprises a modified nucleic acid. In some embodiments, the modified nucleic acid is a conformationally-stabilized nucleic acid. In some embodiments, the modified nucleic acid is selected from the group consisting of: PNA, LNA, modified DNA, and BNA.

In some embodiments, the fusion molecule comprises an antigen or antibody. In some embodiments, the polymer scaffold forms a complex comprising said polymer scaffold bound to said fusion molecule bound to said target analyte in the presence of said target analyte, and wherein said complex is adapted to translocate through said nanopore from the first volume to the second volume under an applied voltage.

In some embodiments, the label comprises a modified nucleic acid. In some embodiments, the modified nucleic acid is a conformationally stabilized nucleic acid. In some embodiments, the modified nucleic acid is selected from the group consisting of: PNA, LNA, BNA, RNA, and DNA

In some embodiments, the label comprises a molecule selected from the group consisting of: PEG, protein, antibody, DNA, and structured DNA.

In some embodiments, the polymer scaffold comprises dsDNA. In some embodiments, the polymer scaffold comprises at least one fusion molecule binding domain capable of binding to the fusion molecule. In some embodiments, the polymer scaffold comprises at least one label binding domain capable of binding to the label.

In some embodiments, the fusion molecule comprises a scaffold binding domain capable of binding to the polymer scaffold at a first target. In some embodiments, the label comprises a scaffold binding domain capable of binding to the polymer scaffold at a second target

In some embodiments, the fusion molecule provides a unique and detectable electrical signal in a target analyte-bound state as compared to a target analyte-unbound state upon translocation through the nanopore when bound to said polymer scaffold. In some embodiments, the fusion molecule comprises PNA bound to a molecule comprising a target binding moiety. In some embodiments, the molecule comprising a target binding moiety comprises an antibody, an aptamer, an antibody fragment, an affibody, a nanobody, an epitope, a hormone, a neurotransmitter, a cytokine, a growth factor, a cell recognition molecule, a nucleic acid, a peptide, a chemical group, chemical modification, or a receptor.

In some embodiments, the target analyte comprises a protein, a peptide, a polynucleotide, a hormone, steroid, intra/extra cellular vesicle, liposome, endosome, nucleated or enucleated cell, mitochondria, virus, viral particle, bacterium, a chemical compound, an ion, or an element.

In some embodiments, the mixed sample comprises an environmental sample or a biological sample. In some embodiments, the mixed sample comprises whole blood, red blood cells, white blood cells, hair, nails, swabs, urine, sputum, saliva, semen, lymphatic fluid, amniotic fluid, cerebrospinal fluid, peritoneal effusions, pleural effusions, fluid from cysts, synovial fluid, vitreous humor, aqueous humor, bursa fluid, eye washes, eye aspirates, plasma, serum, pulmonary lavage, lung aspirates, liver, spleen, kidney, lung, intestine, brain, heart, muscle, pancreas, primary cell lines, secondary cell lines, or any combination thereof. In some embodiments, the mixed sample comprises food, water, soil, or waste.

In some embodiments, the device comprises at least two nanopores in series, and wherein said polymer scaffold is simultaneously in said at least two nanopores during translocation.

In an embodiment, the present disclosure provides a method for detecting a target analyte suspected to be present in a mixed sample, the method comprising: (a) loading a polymer scaffold, a fusion molecule or compound, a label, and a mixed sample suspected to contain a target analyte into a device comprising a nanopore that separates an interior space of the device into two volumes, under conditions that allow said label to bind to said polymer scaffold, that allow said fusion molecule or compound to bind to said polymer scaffold, and that allow said fusion molecule or compound to bind to said target analyte, wherein said polymer scaffold comprises at least one fusion molecule binding domain capable of binding to the fusion molecule or compound, wherein said polymer scaffold comprises at least one label or label binding domain capable of binding to the label, wherein said fusion molecule or compound comprises a target binding domain capable of binding to the target analyte, and wherein said fusion molecule comprises a scaffold binding domain capable of binding to the polymer scaffold at a first target, and wherein said label comprises a scaffold binding domain capable of binding to the polymer scaffold at a second target; (b) configuring the device to pass the polymer scaffold in any orientation through the nanopore from one volume to the other volume; and (c) collecting an electrical signal correlated to passage of said polymeric scaffold in any orientation through the nanopore.

In certain embodiments, the polymer scaffold is dsDNA. In some embodiments, the polymer scaffold has a plurality of ordered label binding domains for increased resolution of detection of the polymer scaffold in a bulk sample with a plurality of background molecules. In some embodiments, the polymer scaffold has a plurality of unique fusion molecule binding domains to allow multiplexing of target detection.

In some embodiments, the fusion molecule provides a provides a unique and detectable electrical signal in a bound state as compared to an unbound state upon translocation through the nanopore when bound to said polymer scaffold. In some embodiments, the fusion molecule comprises PNA bound to a molecule comprising a target binding moiety. In some embodiments, the fusion molecule comprises nucleic acid, or modified nucleic acid (e.g. LNA, BNA, or any other XNA, where X=modified base), peptide, protein, aptamer, DNA, or RNA, or small molecule or chemical group bound to a molecule comprising a target binding moiety. In some embodiments, the modified nucleotide is a conformationally or sterically stabilized nucleic acid (e.g., PNA, LNA, or BNA). In some embodiments the fusion molecule creates a triplex formation. In some embodiments the fusion molecule is in duplex formation with a scaffold.

In some embodiments, the label comprises PNA LNA, BNA, or another XNA (X=base modification). In some embodiments, the label comprises modified nucleic acid, peptide, protein, aptamer, DNA, or RNA, or small molecule or chemical group. In some embodiments the label creates a triplex formation. In some embodiments the fusion molecule is in duplex formation with a scaffold. In certain embodiments, the PNA is bound to a detectable tag, such as a PEG. In certain embodiments, the size, shape, and or charge of the detectable tag can be modified to increase resolution based on current impedance (or equivalent signals) in a pore of a specific shape or size.

Also provided are methods of analyzing data from a nanopore device to detect the presence of a target analyte in a mixed sample, the method comprising: (a) obtaining an electrical signal from an event generated by a nanopore analysis of a mixture, wherein said mixture comprises a sample suspected of containing a target analyte, a polymer scaffold comprising at least one fusion molecule binding domain and at least one label binding domain or label, and a fusion molecule capable of binding said fusion molecule binding domain and said target analyte; (b) analyzing said electrical signal to detect the presence of a first signature curve indicating detection of a label bound to the polymer scaffold; and (c) analyzing said electrical signal to detect the presence of a second signature curve indicating detection of a target analyte bound to said polymer scaffold.

Also provided are compositions for enhancing detection of analytes form a mixed sample using a nanopore. Thus, in an embodiment, provided is a polymeric scaffold comprising at least one fusion molecule binding domain and at least one label binding domain or label. In certain embodiments, the polymeric scaffold comprises a plurality of fusion molecule binding domains for multiplex analysis of analytes. In other embodiments, the polymeric scaffold comprises a plurality of fusion molecule binding domains for increased resolution of detection of a single analyte. In an embodiment, the polymeric scaffold comprises a plurality of label binding domains for increased resolution of identification of the polymeric scaffold.

In some embodiments, provided is a polymeric scaffold bound to a plurality of probes. In an embodiment, the probe is a fusion molecule. In another embodiment, the probe is a label. In some embodiments, the fusion molecule has a target analyte binding moiety. In some embodiments, the fusion molecule is bound to the polymeric scaffold and to a target analyte. In some embodiments, the fusion molecule is bound to the target analyte through an intermediary.

Also provided are methods of encoding one or more bit(s) of information by placing one or more molecules along a polymer in such a fashion that the original information can be retrieved by passing the polymer through a nanopore and examining the current impedance signatures curves.

Also provided are kits, packages or mixtures that detect the presence of a target molecule or particle. In an embodiment, the kit comprises a polymer scaffold comprising at least one fusion molecule binding domain and at least one label binding domain, a label capable of binding to said binding domain, and a fusion molecule capable of binding to a target ligand and to said fusion molecule binding domain. In some aspects, the kit, package or mixture further comprises a sample suspected of containing the target molecule or particle. In some aspects, the sample further comprises a detectable label capable of binding to the target molecule, particle, ligand/target complex, or ligand/particle complex.

Also provided are method of analyzing data to detect the presence of a target analyte in a mixed sample, comprising (a) obtaining an electrical signal from an event generated by a nanopore analysis of a mixture, wherein said mixture comprises a sample suspected of containing a target analyte, a polymer scaffold comprising at least one fusion molecule binding domain and at least one label binding domain or label, and a fusion molecule capable of binding said fusion molecule binding domain and said target analyte; (b) analyzing said electrical signal to detect the presence of a first signature curve indicating detection of a label bound to the polymer scaffold; and (c) analyzing said electrical signal to detect the presence of a second signature curve indicating detection of a target analyte bound to said polymer scaffold.

In addition, provided is a method for identifying binding sequences on a polymer scaffold, comprising: (a) providing a polymer scaffold comprising a label binding domain; (b) loading said polymer scaffold and a label adapted to bind to said label binding domain into a device comprising a nanopore that separates an interior space of the device into two volumes, under conditions that allow said label to bind to said label binding sequence; (c) configuring the device to pass the polymer scaffold through the nanopore from one volume to the other volume; and (d) collecting an electrical signal correlated to passage of said polymeric scaffold through the nanopore.

Also provided are kits, packages or mixtures to store and/or read information on a polymer scaffold. In an embodiment, the kit comprises two or more labels each having different size, charge and/or shape and a polymer scaffold encoding information to be read. In some embodiments, the kit further comprises a nanopore device comprising a nanopore that separates and connects two volumes in the nanopore device, wherein the nanopore device is adapted to identify each of the labels when the label is bound to said polymeric scaffold and said polymeric scaffold translocates through said nanopore.

### BRIEF DESCRIPTION OF THE DRAWINGS

Provided as embodiments of this disclosure are drawings that illustrate features by exemplification only, and not limitation.

FIG. 1 illustrates how a nanopore is adapted to detect ligands bound to a nucleotide.

FIGS. 2A and 2B shows a PNA ligand that has been modified as to increase ligand size, and therefore facilitate detection. FIG. 2C shows a DNA scaffold that contains a reactive moiety and conjugates to a molecule that has compatible reactivity for covalent coupling

FIG. 3A illustrates the detection of a target molecule or particle with fusion molecules according to an embodiment of the method. FIG. 3B shows a fusion molecule that has an antibody analyte capture domain fused to a Azide reactive group through a PEG linker.

FIG. 4 shows representative and idealized current profiles of three example molecules, demonstrating that binding between a target molecule (or particle) and a fusion molecule can be detected when passing through a nanopore, since it has a different current profile, compared to that of the fusion molecule alone or the DNA alone. Specifically, FIG. 4A shows current profiles consistent with higher salt concentrations (>0.4 M KCl, for example at 1M KCl) in the experimental buffer and a positive applied voltage, generating a positive current flow through the pore. By another example, FIG. 4B shows current profiles consistent with lower salt concentrations (<0.4 M KCl, for example at 100 mM KCl) in the experimental buffer and again at a positive applied voltage. By another example, FIG. 4C shows current profiles consistent with lower salt concentrations (<0.4 M KCl, for example at 100 mM KCl) in the experimental buffer and a negative applied voltage.

FIG. 5 illustrates the multiplexing capability of the present technology by including different binding motifs in the polymer scaffold. Such multiplexing can be accomplished with one nanopore or more than one nanopore.

FIG. 6 provides the illustration of a more specific example, where a double-stranded DNA is used as the polymer scaffold, and a human immunodeficiency virus (HIV) envelope protein is used as the ligand. The combination is used to detect an anti-HIV antibody.

FIG. 7 illustrates a nanopore device with at least two pores separating multiple chambers. Specifically, FIG. 7A is a schematic of a dual-pore chip and a dual-amplifier electronics configuration for independent voltage control (V₁ or V₂) and current measurement (l₁, or l₂) of each pore. Three chambers, A-C, are shown and are volumetrically separated except by common pores. FIG. 7B is a schematic where electrically, V₁ and V₂ are principally applied across the resistance of each nanopore by constructing a device that minimizes all access resistances to effectively decouple l₂ and l₂. FIG. 7C depicts a schematic in which competing voltages are used for control, with arrows showing the direction of each voltage force.

FIG. 8 illustrates a nanopore device having one pore connecting two chambers and example results from its use. Specifically, panel (a) depicts a schematic diagram of the nanopore device. Panel (b) depicts a representative current trace showing a blockade event resulting from the passage of a double-stranded DNA passing through the pore. The current amplitude shift amount (Δl=l₀−l_{B}) and duration to are used to quantify the passage event. Panel (c) depicts a scatter plot showing the change in current amount (Δl) vs. translocation time (t_{D}) for all blockade events recorded over 16 minutes.

FIG. 9 depict current traces measured within an embodiment of a nanopore device fabricated in accordance with the present invention. The provided current traces show that unbound dsDNA causes current enhancement events at KCl concentrations below 0.4 M. Current enhancements appeared as downward shifts in the provided experiment, since the voltage and current are both negative (as in FIG. 3C). Specifically, in DNA alone control experiments using a 10-11 nm diameter pore in 0.1M KCl at −200 mV, 5.6 kb dsDNA scaffold (panel (a)) causes brief current enhancement events that are 50-70 pA in amplitude and 10-200 microseconds in duration. Likewise, 48 kb Lambda DNA (panel (b)) causes current enhancement events 50-70 pA in amplitude and 50-2000 microseconds in duration.

FIG. 10 illustrates a gel showing the sequence specificity of binding of a bisPNA to a dsDNA polymer scaffold.

FIG. 11 shows representative electrical signals from nanopore detection of a polymer scaffold (panel (a)) not bound to bisPNA, and panels (b), (c) bound to bisPNA.

FIG. 12 is a gel showing binding of PNA without (lane 2) or with a detectable tag of PEG 5 k (lane 3) or PEG 10 k (lane 4).

FIG. 13 shows representative electrical signals from nanopore detection of a polymer scaffold bound to (panel (a)) PNA alone, (panel (b)) PNA with a PEG 5 k detectable tag, or (panel (c)) PNA with a PEG 10 k detectable tag.

FIG. 14 shows the results of a gel shift assay shows that a single (lane 3) or two (lane 4) gammaPNA-PEG 5 kDa can bind to the same fragment molecule.

FIG. 15 shows the results of a gel shift assay shows that one (lane 3) or two (lane 4) monostrepatavidin proteins can bind to a single dsDNA polymer scaffold with multiple label (monostreptavidin) binding sites.

FIG. 16 illustrates detection of multiple labels on a dsDNA scaffold. Panel (a) shows a gel shift assay. Panel (a) is an image from a DNA-(PNA-biotin)-Neutravidin (DPN) EMSA in labeling buffer, with the following lanes (left to right): sizing ladder with top rung 5 kb; 5.6 kb DNA only; DNA-PNA with 3×, 7×, 16× and 36× excess Neutravidin to biotin; and DNA-PNA. Panel (b) is a schematic of one PNA-biotin-Neutravidin region on the 5.6 kb dsDNA scaffold, and a representative translocation event recorded from each of three consecutive experiments using the same pore at 200 mV in 1M KCl: DNA alone, Neutravidin alone, and then DPN complexes with 10× excess Neutravidin to biotin. Panel (c) is a scatter plot of ΔG versus duration for the three consecutive experiments (D, N, and DPN). Panel (d) is a horizontal probability histogram of ΔG for the three data sets, with the inset histogram for the 578 DPN events with duration longer than 0.08 ms.

FIG. 17 shows a prototype illustration of an electrical signal generated upon the translocation of a polymer scaffold with PNA molecules bound to 5K PEGs on either end of the polymer scaffold, with a fusion molecules and target analyte in the middle, through the nanopore.

FIG. 18 shows a dsDNA scaffold with events 0.1-0.5 ms, and with a single antibody acting as a label at one end, and the absence or presence of a separate target analyte antibody at the other end. Event signatures have a single “spike” when only the label antibody is present, and two “spikes” when the target analyte antibody is present, signaling detection of the target for that molecule.

FIG. 19 shows a dsDNA scaffold with events 0.5-10 ms, and with a single antibody acting as a label at one end, and the absence or presence of a separate target analyte antibody at the other end. Event signatures have a single “spike” when only the label antibody is present, and two “spikes” when the target analyte antibody is present, signaling detection of the target for that molecule.

FIGS. 20A and 20B depict reverse phase high-performance liquid chromatography (RP-HPLC) chromatograms of reagents used in the PNA-PEG conjugations.

FIG. 21 shows the absorbance trace at 270 nm of the reaction products that result from the incubation of PNA with a 10 kDa PEG molecule. Peaks at this absorbance indicate the presence of PNA.

FIG. 22 shows the MALDI-TOF mass spectra of the PNA molecule alone, indicating a molar mass of 7860.365 daltons.

FIG. 23 depicts the MALDI-TOF mass spectra of the reaction product that results from the reaction of PNA with a 10 kDa PEG as shown on RP-HPLC (FIG. 21, 37.8 min). The product shows a broad mass ranging from approximately 18,500 Da to 20,200 Da. A broad peak is indicative of a polydisperse PEG.

FIGS. 24A and 24B depict the gel shift assays between 550 bp DNA and purified PNA-PEG or PNA-HIV peptide conjugates.

FIG. 25 shows an EMSA assay with DNA that has been invaded by PNA-HIV peptide and subsequently titrated with an increasing amount of HIV antibody. All of the DNA-PNA-HIV bait reagent is fully bound with antibody at a 5-fold molar excess of antibody.

FIG. 26 shows the 3250 bp DNA scaffold with single PNA-PEG payload site location, and the 5631 bp DNA scaffold with two PNA-PEG payload site locations.

FIG. 27 shows event plots and histograms for 3250 bp DNA without (black squares) and with (blue circles) a single PNA-PEG payload, passing through a ˜23 nm diameter nanopore.

FIG. 28 shows an all point histogram for all events shown in FIG. 27.

FIG. 29 shows the percentage of events in FIG. 27 with a max ΔG>3 nS.

FIG. 30 shows all N=1 level events from the 3250 bp DNA alone experiment, comprised of fully folded and unfolded translocation events through the pore.

FIG. 31 shows all N=2 level events from the 3250 bp DNA alone experiment, comprised of partially folded events with a first level in the folded state and a second level in the unfolded state (second level shows depth vs. cumulative duration).

FIG. 32 shows one of only five N=3 level events from the 3250 bp DNA alone experiment.

FIG. 33 compares the minimum sample within N=1 level events for the 3250 bp DNA alone experiment and for the 3250 bp DNA with PNA-PEG experiment.

FIG. 34 shows N=2 level events from the 3250 bp DNA with PNA-PEG experiment, with a fraction for which the third level (a terminal “spike”) is often missed by the algorithm, and other events in which a terminal “spike” signaling the payload is the second level.

FIG. 35 shows three representative events from the set of N=2 level events from the 3250 bp DNA with PNA-PEG experiment, showing a third level (consistent with the payload) that the algorithm failed to detect.

FIG. 36 shows representative N=3 level events from the DNA-PNA-PEG reagent experiment.

FIG. 37 shows all N=3 level identified events from the DNA-PNA-PEG reagent experiment.

FIG. 38 shows the event plots and histograms for 5631 bp DNA without (red diamonds) and with PNA-PEG (10 kDa) at 10× (squares) and 25× (circles) the number of sites (2 sites per DNA, FIG. 26), sequentially tested on the same 18-19 nm diameter pore.

FIG. 39 shows an all point histogram for all events shown in FIG. 38.

FIG. 40 shows the percentage of events in FIG. 38 with a max ΔG>3 nS.

FIG. 41 shows all N=2 level identified events from the 5.6 kb DNA experiment.

FIG. 42 shows all N=2 level identified events from the 5.6 kb DNA with 10×PNA-PEG experiment.

FIG. 43 shows the rare (4.3% or total) N=3 level identified events from the 5.6 kb DNA experiment.

FIG. 44 shows representative events with N=1, 2, 3, 4 and 5 levels identified from the 5.6 kb DNA with 10×PNA-PEG experiment.

FIG. 45 shows all N=3 level identified events (19.3% of total) from the 5.6 kb DNA with 10×PNA-PEG experiment.

FIG. 46 shows all N=4 level identified events (11.8% of total) from the 5.6 kb DNA with 10×PNA-PEG experiment.

FIG. 47 shows all N=5 level identified events (6.2% of total) from the 5.6 kb DNA with 10×PNA-PEG experiment.

FIG. 48 shows the breakdown (by percentage) of the events by number of identified levels, for DNA alone, DNA with 10×PNA-PEG and DNA with 25×PNA-PEG data sets.

FIGS. 49A and 49B shows the FT-ICR-MS profile of a maleimide-tagged 10 kDa PEG molecule.

FIGS. 50A, 50B, and 50C depicts the results of a gel-shift analysis of 3250 bp DNA with and without a PNA-PEG on a Tapestation 2200 instrument.

FIGS. 51A and 51B demonstrate the absorbance trace of LNA at 260 nm before and after reduction by TCEP.

FIG. 52 depicts the absorbance profile of 10 kDa PEG alone upon elution in reverse phase chromatography.

FIG. 53 shows the absorbance profiles at 260 nm of the reaction products between LNA and a 10 kDa PEG by HPLC. An intense, new peak is observed 20.34 min, while the reduced peak previously seen at 12.74 min is no longer present, indicating successful conjugation of LNA to the 10 kDa PEG.

FIG. 54 shows schematics of complexes with representative nanopore event signatures, an electromobility shift assay (EMSA) and nanopore event plots (middle), all showing that only the bulkier HIV Ab-bound scaffold/fusion complex generates deeper and longer event signatures.

FIG. 55 shows the event plots and histograms for 5631 bp DNA alone (circle), DNA with 10×PNA-PEG (PP) (square and diamond), DNA with 10×PP and with 10×PNA-peptide for the V3 loop (PV3B) (triangle), and DNA with 10×PP, 10×PV3B and 2×HIV Ab (star), sequentially tested on the same 24.5-25.5 nm diameter pore.

FIG. 56 shows an all point histogram for all events shown in FIG. 55.

FIG. 57 shows the percentage of events in FIG. 55 with a max ΔG>3 nS.

FIG. 58 shows the breakdown (by percentage) of the events by number of identified levels for the reagents displayed (data from FIG. 55).

FIGS. 59A and 59B shows representative events with N=3 and 4 levels identified, respectively, from 5.6 kb DNA with 10×PNA-PEG (PP) and from DNA with 10×PNA-PEG (PP) and 10×PNA-peptide (PV3B).

FIG. 60 shows A) a typical HIV Ab-bound event, with N=4 detected levels (after optimization-based fitting), and B) HIV Ab-bound events with N=6 detected levels for which the antibody-bound level(s) and total event times were faster than normal.

FIG. 61 shows all N=4 level identified events (15.6% of total) from the 5.6 kb DNA with 10×PNA-PEG and 10×PNA-peptide.

FIG. 62 shows all N=4 level identified events (14% of total) from the 5.6 kb DNA with 10×PNA-PEG and 10×PNA-peptide and 2×HIV antibody.

FIG. 63 depicts the HPLC trace of the BNA molecule alone at an absorbance wavelength of 260 nm.

FIG. 64 depicts the HPLC trace of the reaction mixture of BNA and a 10 kDa PEG at an absorbance wavelength of 260 nm.

FIG. 65 shows the HPLC trace of the HIV peptide alone at an absorbance wavelength of 260 nm.

FIG. 66 shows the HPLC trace of the reaction mixture of LNA and the HIV peptide at an absorbance of 260 nm.

FIG. 67 depicts a schematic of the hybridization assay of an LNA or BNA based probe with competing oligonucleotides to a dsDNA fragment.

FIGS. 68A, 68B, and 68C depict the change in electrophoretic mobility of DNA in a hybridization approach with labeled LNA probes. Specifically, FIG. 68A shows the gel shift exhibited by a hybridization assay complete with an LNA-HIV conjugate with and without downstream labeling with an HIV-specific antibody. FIG. 68B demonstrates the shift in mobility between a biotin labeled LNA probe, and one that has also been subsequently labeled with monostreptavidin. FIG. 68C shows the change in mobility between an unlabeled LNA probe, and one that was previously conjugated to 10 kDa PEG.

Some or all of the figures are schematic representations for exemplification; hence, they do not necessarily depict the actual relative sizes or locations of the elements shown. The figures are presented for the purpose of illustrating one or more embodiments with the explicit understanding that they will not be used to limit the scope or the meaning of the claims that follow below.

### DETAILED DESCRIPTION

Throughout this application, the text refers to various embodiments of the present devices, compositions, systems, and methods. The various embodiments described are meant to provide a variety of illustrative examples and should not be construed as descriptions of alternative species. Rather, it should be noted that the descriptions of various embodiments provided may be of overlapping scope. The embodiments discussed herein are merely illustrative and are not meant to limit the scope of the present invention.

Also throughout this disclosure, various publications, patents and published patent specifications are referenced by an identifying citation. The disclosures of these publications, patents and published patent specifications are hereby incorporated by reference into the present disclosure in their entireties.

As used in the specification and claims, the singular form “a”, “an” and “the” include plural references unless the context clearly dictates otherwise. For example, the term “an electrode” includes a plurality of electrodes, including mixtures thereof.

As used herein, the term “comprising” is intended to mean that the systems, devices, and methods include the recited components or steps, but not excluding others. “Consisting essentially of” when used to define systems, devices, and methods, shall mean excluding other components or steps of any essential significance to the combination. “Consisting of” shall mean excluding other components or steps. Embodiments defined by each of these transition terms are within the scope of this invention.

All numerical designations, e.g., distance, size, temperature, time, voltage and concentration, including ranges, are approximations which are varied (+) or (−) by increments of 0.1. It is to be understood, although not always explicitly stated that all numerical designations are preceded by the term “about”. It also is to be understood, although not always explicitly stated, that the components described herein are merely exemplary, and that equivalents of such are known in the art.

As used herein, “a device comprising a nanopore that separates an interior space” shall refer to a device having a pore that comprises an opening within a structure, the structure separating an interior space into more than one volume or chamber.

As used herein, the term “scaffold” or “polymer scaffold” refers to a charged polymer capable of binding probes (e.g., labels, payload molecules, or fusion molecules) and translocating through a pore upon application of voltage. In some aspects, the polymer scaffold comprises a deoxyribonucleic acid (DNA), a ribonucleic acid (RNA), a peptide nucleic acid (PNA), a DNA/RNA hybrid, or a polypeptide. The scaffold can also be a chemically synthesized polymer, and not a naturally occurring or biological molecule. In a preferred embodiment, the polymer scaffold is dsDNA to allow more predictable signals upon translocation through the nanopore and reduce secondary structure present in ssDNA or RNA. The polymer scaffold comprises probe binding domains, e.g., label binding domains and/or fusion molecule binding domains. These domains can reside on the ends of the DNA as chemical modification to which labels or analyte detection molecules are chemically tethered or bound. These domains can reside within the scaffold as a base or series of bases, or a chemically modified bases or bases.

As used herein, the term “binding domain” when referring to a segment on the polymer scaffold, e.g., a fusion molecule binding domain or a label binding domain, refers to a domain that binds under relaxed to stringent conditions to another molecule or compound. In the case of several embodiments of this invention, the binding domain comprises a specific sequence on the polymer scaffold which binds to a probe. Other embodiments, the binding domain is a modification to the end of the scaffold to enable probe attachment or binding. In some embodiments, the binding domain is a base or series of bases, or a chemically modified bases or bases. The address/bit location of each binding domain can be determined by detection of the binding of the probes to the polymer scaffold in a nanopore device.

As used herein, the term “probes” refers to molecules or compounds that bind to a binding domain on or at the terminal ends of a polymer scaffold. In several embodiments of this invention, the probes are fusion molecules or compounds, or labels.

As used herein, the term “labels” refer to molecules or compounds that bind to a specific label binding domain on the polymer scaffold or at the terminal ends of a polymer scaffold. Labels are also referred to herein as “payload” molecules. These compounds are adapted to be detectable by a nanopore by measuring current impedance. In preferred embodiments, labels can comprise a molecule for binding to the polymer scaffold, such as a PNA molecule, bound to one or more“detectable tags” which are detectable in a nanopore when bound to a polymer scaffold due to their size, shape, hydrophobicity, hydrophilicity, or charge providing a detectable effect on current impedance. Thus, the detectable tag can be used to enhance resolution of detection of the label in a nanopore or to provide unique characteristics for identification in a nanopore via a unique electrical signal. The detectable tag can be bound, either covalently or non-covalently, to the a molecule comprising a polymer-scaffold binding domain, or can bind directly to the polymer scaffold.

As used herein, the term “fusion molecule” refers to molecules or compounds that bind to a specific fusion molecule binding domain on, or bind or react with chemical groups at the termini of a polymer scaffold, and also bind to a target analyte. Upon translocation through the nanopore, a fusion molecule bound to the polymer scaffold can generate an electrical signal that is capable of discriminating whether or not the fusion molecule is bound to a target analyte. In this way, a target analyte in a solution can be detected and/or quantified.

As used herein, the term “target analyte” refers to a molecule, compound, virus, cell, or other entity of interest to be detected in a sample. The target analyte may be detected by binding to an analyte binding domain on a fusion molecule bound to a polymer scaffold that translocates through a nanopore, providing a defined electrical signal.

As used herein, the term “electrical signature” encompasses a series of data collected on current, impedance/resistance, or voltage over time depending on configuration of the electronic circuitry. Conventionally, current is measured in a “voltage clamp” configuration; voltage is measured in a “current clamp” configuration, and resistance measurements can be derived in either configuration using Ohm's law V=IR. Impedance can also be generated by measured from current or voltage data collected from the nanopore device. Types of electrical signals referenced herein include current signatures and current impedance signatures, although various other electrical signatures may be used to detect particles in a nanopore.

As used herein, the term “nanopore” refers to an opening (hole or channel) of sufficient size to allow the passage of particularly sized polymers. Voltage is applied to drive negatively charged polymers through the nanopore.

As used herein, the term “sensor” refers to a device that collects a signal from a nanopore device. In many embodiments, the sensor includes a pair of electrodes placed at two sides of a pore to measure an ionic current across the pore when a molecule or other entity, in particular a polymer scaffold, moves through the pore. In addition to the electrodes, an additional sensor, e.g., an optical sensor, may be to detect an optical signal in the nanopore device. Other sensors may be used to detect such properties as current blockade, electron tunneling current, charge-induced field effect, nanopore transit time, optical signal, light scattering, and plasmon resonance.

As used herein, the term “current measurement” refers to a series of measurements of current flow at an applied voltage through the nanopore over time. The current is expressed as an x,y value where x represents a point in time, and y represents the amount of current impeded in the channel. Current measurement is an electrical signal related to current impedance/resistance and voltage (other electrical signals) through Ohm's law.

As used herein, the term “open channel” refers to the baseline level of current through a nanopore channel within a noise range where the current does not deviate from a threshold of value defined by the analysis software.

As used herein, the term “event” refers to a set of current impedance measurements that begins when the y value of a current measurement deviates from the open channel value by a defined threshold, and ends when the y value returns to within a threshold of the open channel value.

As used herein, the term “current impedance signature” refers to a collection of current measurements where the first such measurement begins when the value of y exceeds a given threshold defined by the software, and ends when the value of y returns past that same threshold. This threshold may be used to identify multiple signatures within an event (i.e., since a polymer may have one or more molecules bound to it, an event may contain one or more signatures).

As used herein, the term “signature curve” refers to the product of a mathematical formula applied to all the x,y points in a single signature. This formula may be as simple as a simple average of all the points (yielding a single line at y), or as a moving average of every N number of points (yielding a simple curve), or another mathematical formula. Step fitting algorithms are another example of a formula to apply to each signature. The number of steps or their properties can be used to infer properties about the signature curve or curves. (See, e.g., C Raillon, P Granjon, M Graf, L J Steinbock, and A Radenovic. Fast and automatic processing of multi-level events in nanopore translocation experiments. Nanoscale, 4(16):4916, 2012, incorporated by reference in entirety). Since nanopores are inherently non-deterministic, electrical signals may vary considerably each time the same type of molecule passes through. Therefore, the software that analyzes measurements may employ enough flexibility to assure a consistent signature curve each time the same molecule is read.

As used herein, the term “optical sensor” refers to an apparatus that captures light within a fixed field of view that may reside at or adjacent to the nanopore.

As used herein, the term “optical event” refers to a set of optical measurements captured by the sensor from a single polymer that may contain one or more tagged molecules. Because the sensor cannot discern between the beginning and end of a polymer using optics, the ends of the polymer may be detected by using current impedance measurements to determine when a polymer enters (when the measurement's y value exceeds the open channel threshold, or by adding tagged molecules that will produce a known optical measurement to the each end of the polymer.

As used herein, the term “optical measurement” refers to a value obtained by that optical sensor within a fixed period of time. This measurement may include, but not be limited to, one or more of individual values, such as color, luminescence, and intensity.

As used herein, the term “symbol” refers to the assembly of one or more optical signatures within an event so as to comprise a single abstraction. E.g., “red, green, red, green” may equate to the letter “A.”

### Polymer Scaffold Binding and Identification

The present disclosure provides methods and systems for identification of polymer scaffolds comprising target analyte binding domains in a nanopore. The methods and systems can also be adapted to measure the affinity of a molecule binding with another molecule. Further, such detection, quantitation, and measurement can be carried out in a multiplexed manner, greatly increasing its efficiency.

The present disclosure, in an embodiment, provides devices and methods for identifying a polymer scaffold, such as a DNA, RNA, PNA or polypeptide molecule, using a nanopore. The methods employ a plurality of detectable labels that specifically bind to a particular sequence (referred to as a “label binding domain”) on the polymer scaffold. The labels can differ from each other by size, shape, hydrophobicity, hydrophilicity, or charge. Therefore, when a polymer scaffold bound to a set of labels is passed through a suitably configured nanopore, the labels can be identified or at least distinguished from each other by measuring the current impedance as each label passes through the nanopore. Orientation of the polymer scaffold as it translocates through the nanopore is not limited to a specific direction, as electrical signals for labels may be identified based on translocation in either orientation.

By virtue of the binding specificity between the detectable labels and the label binding domains, the relative locations and order of the label binding domains on the polymer scaffold can be derived from the bound labels that generate a unique current impedance in the nanopore. Thus, the nanopore device does not need to identify each monomer of the entire polymer scaffold or even a portion of the polymer scaffold. Therefore, if a polymer scaffold is encoded with information in a format of sequences of label binding domains, the detection of the labels bound to the label binding domains “decodes” such information.

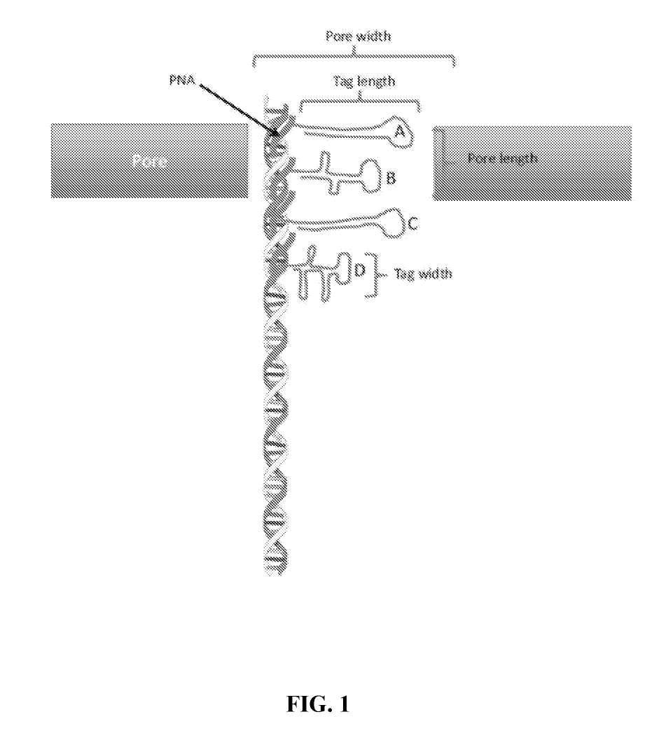

As illustrated in FIG. 1, in certain embodiments, labels A, B, C and D each specifically binds to a label binding domain on a DNA molecule. In the embodiment shown, each label comprises a PNA molecule bound to a detectable tag. These labels can be identified and distinguished from each other by their current impedance when passing through the nanopore. This current impedance is affected by width, length, size, hydrophobicity, and/or charge of the label. In the embodiment provided in FIG. 1, these parameters are determined by the width, length, size, and/or charge of the detectable tag bound to the PNA molecule. Thus, each label may provide a unique electrical signal upon passage through the nanopore, allowing identification of each label bound to the polymer scaffold and therefore to each label binding domain present on the polymer scaffold. The PNA molecule comprises a sequence complementary to the label binding domain on the double-stranded DNA. Identification of the labels shown in FIG. 1 leads to identification of the sequence of label binding domains, A-B-C-D. If the polymer scaffold entered into the opposite orientation, the sequence would still be detected as D-C-B-A, and provide information regarding the location and identity of the label binding domains on the polymer scaffold. In practice, the labels will likely be spaced apart more than they appear in the figure. On the order of 10 s to 100 s to 1000 s of basepairs apart. Furthermore, positioning the labels close, more than one label can be present in the pore, with each contributing to a unique event signature, providing greater breath of data encoding by a set of molecules, e.g. AB is different from A or B.

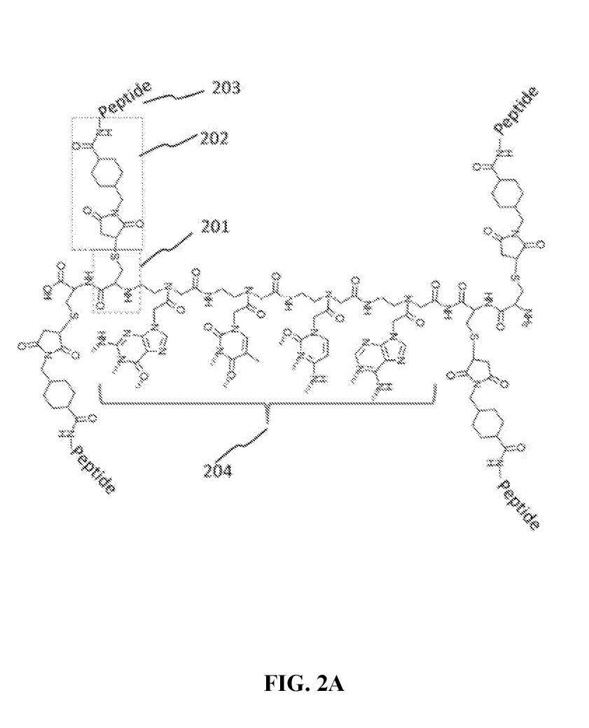

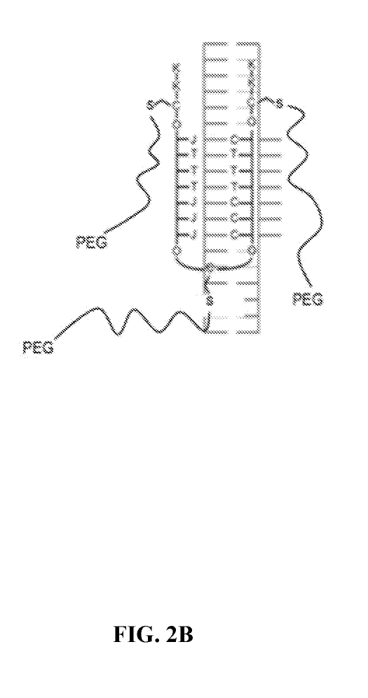

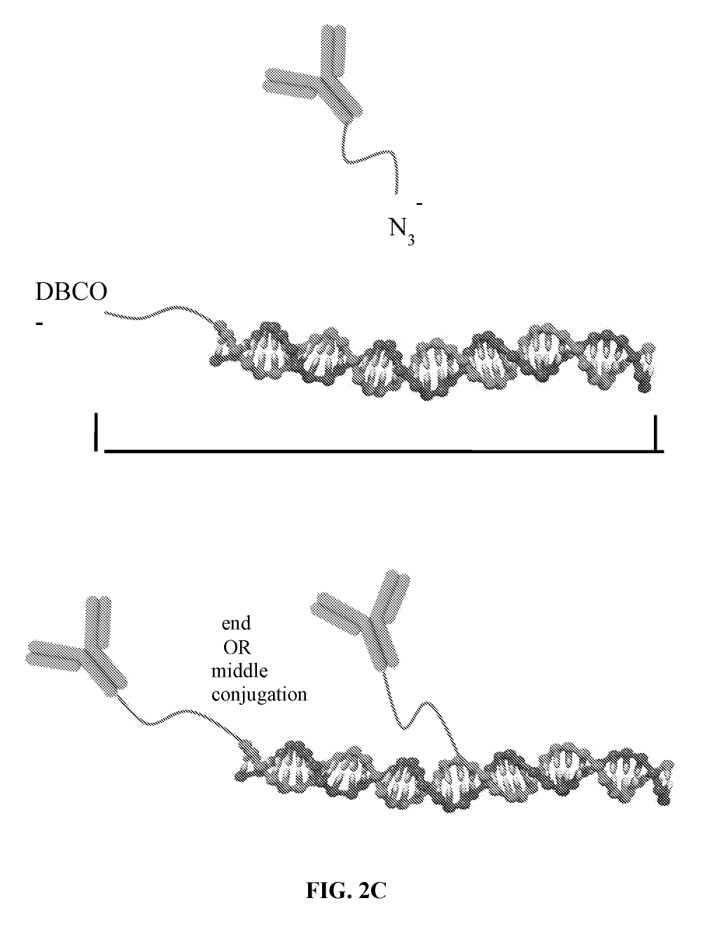

Since the labels can be modified by parameters such as width, length, size, hydrophobicity and/or charge, the compositions and methods described herein can be performed with pores of varying size, including larger pores, which are easier and cheaper to manufacture than smaller nanopore devices. For example, FIG. 2 shows a PNA label that has been modified by addition of a detectable tag (in peptides (FIG. 2A), and in polyethylene glycol (FIG. 2B) so as to increase its size, and therefore facilitate detection. This greater size results in a greater change in current flow through the pore, or current impedance, compared to an unlabeled PNA. FIG. 2C demonstrates a method of tagging the scaffold (terminus or within) with a molecule that can act as a label or that can capture analyte.

In an embodiment, therefore, the present technology provides a method for identifying a plurality of label binding domains on a polymer scaffold. The method entails (a) loading a polymer scaffold into a device with a pore that separates and connects two volumes, under conditions that (i) allow a plurality of labels each to specifically bind to one or more of the label binding domains on the polymer scaffold and (ii) allow the polymer scaffold, along with the bound labels, to translocate through the pore from one volume to the other volume, and (b) collecting the electrical signal correlated to the passage of the polymer scaffold through the nanopore. Using the electrical signal, events identifying the translocation of the molecule may be collected and analyzed to identify electrical signals correlated with each label.

An “electrical signal” can include current measurement creating a current signature from the translocation through the pore of one, or alternatively two or more adjacent labels at a time. The identification of multiple labels in an electrical signal may be due to simultaneous location in the nanopore during translocation, or due to sequential location in the nanopore during translocation. When an electrical signal includes only one label, the label needs to be spaced apart from its adjacent labels to avoid the adjacent labels (when all are bound to the polymer scaffold) from interfering with the detection of the label by correlation with the electrical signal.

Due to Brownian motion, the ability to detect a label bound to the polymer scaffold by measuring current impedance changes requires the bound labels to be spaced apart such that each bound label yields a current impedance measurement that is not influenced by neighboring bound labels. Therefore, in some embodiments, the label binding domain is spaced apart from other label binding domains on the polymer scaffold so that only one label is in the pore at a time. For example, if the nanopore is Inm in length, the proper separation may be achieved by having label binding domains separated by a distance of at least 1 nm (e.g., approximately 3 nucleotides (nt)). In other words, two adjacent label binding domains are separated by at least 1 nm (or 3 nt) on the polymer scaffold. This separation may be adjusted depending the length of the nanopore used to detect the labels bound to the polymer scaffold. In some aspects, each label binding domain is separated from an adjacent label binding domain on the polymer scaffold by at least Inm, 2 nm, 3 nm, 4 nm, 5 nm, 6 nm, 7 nm, 8 nm, 9 nm, 10 nm, 15 nm, 20 nm, 25 nm, 30 nm, 40 nm, 50 nm, 100 nm, 200 nm, 300 nm, 400 nm, or 500 nm. In some embodiments, however, sufficient resolution of labels bound to the polymer scaffold may be achieved without separation of label binding domains on the polymer scaffold.

In some embodiments, adjacent labels may be part of a unique electrical signal used for identification of the label binding domains or bound labels. For instance, labels A and B together may provide one unique electrical signal, whereas the same label B and the next label C can jointly form a different unique electrical signal.

In each of these above scenarios, the nanopore device can be suitably configured to identify each unique electrical signal generated by two or more bound labels without interference from other nearby bound labels. For instance, if the unique electrical signal is generated by two adjacent bound labels as the polymer scaffold passes through the nanopore, the nanopore can be long enough to accommodate both labels.

Because labels can include many molecules along the scaffold, one can construct arbitrarily long sequences of unique labels that encode for arbitrary amounts of information, making it possible to use the entire synthetic structure as a data storage mechanism.

### Molecular Detection

The present disclosure provides methods and systems for molecular detection and quantitation of target analytes in a mixed sample. Further, such detection, quantitation, and measurement can be carried out in a multiplexed manner, greatly increasing its efficiency. Methods and compositions for analyte detection are disclosed in PCT Publication WO/2014/182634, incorporated by reference in its entirety.

In a nanopore experiment, a population of current impedance events is generated. Mathematical modeling is used to pick out our target analytes from background within a degree of confidence. However, a mixed sample, e.g., blood that has not been processed, has a large population of background molecules that produce electrical signals that overlap with those of a target. A high error rate may be introduced by these molecules, affecting the reliability of the nanopore to detect target analytes. One mechanism to improve reliability, as disclosed herein, is too attach a label or a sequence of labels to a polymer scaffold to provide a unique electrical signal that can be used to identify the presence and/or identity of a polymer scaffold that has translocated through a nanopore. These polymer scaffolds contain fusion molecule binding domains to bind fusion molecules which bind directly, or through an intermediary, to a target analyte. This detection provides a unique electrical signal upon translocation through the nanopore that can be further discriminated from background molecules by identification of a label or sequence of labels bound to the polymer scaffold. Therefore, provided are improved methods and compositions for detecting target analytes in a bulk sample using a nanopore.

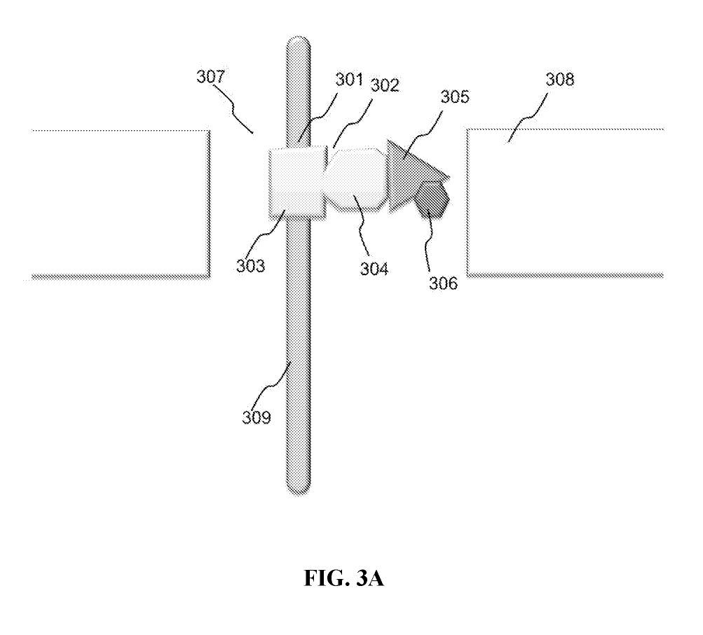

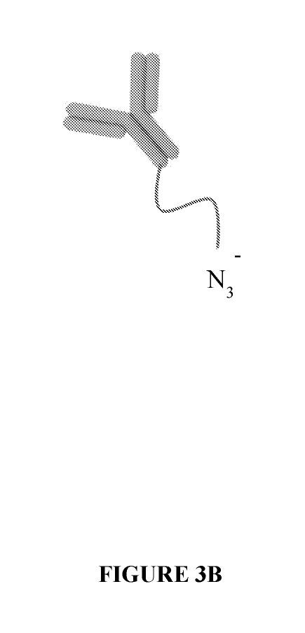

FIG. 3A provides an illustration of an embodiment of the disclosed methods and systems. More specifically, the system includes a ligand comprising an analyte binding moiety 304 that is capable of binding to a target analyte 305 to be detected or quantitated. The ligand 304 can be part of, or be linked to, a scaffold binding moiety (i.e., a “scaffold binding domain”) 303 that is capable of binding to a specific binding motif or fusion molecule binding domain (e.g., a DNA sequence) 301 on a polymer scaffold 309. The ligand, shown in FIG. 3B, can be directly chemically coupled to the scaffold through the binding moiety (as described in [0046] and FIG. 2C). Together, the ligand 304 and the scaffold binding moiety 303 form a fusion molecule 302. In various embodiments, both components of the fusion molecule 302 (i.e., both the ligand 304 and the scaffold binding moiety 303) bind to their respective targets (e.g., target analyte 305 and fusion molecule binding domain 301, respectively) with high affinity and specificity.

Therefore, if all are present in a solution, the fusion molecule 302 binds, on one end, to a polymer scaffold (or simply, “polymer”) 309 through the specific recognition and binding between the fusion molecule binding domain 301 and the scaffold binding moiety 303, and on the other end, to the target analyte 305 by virtue of the interaction between the analyte binding moiety on the ligand 304 and the target analyte 305. Such bindings cause the formation of a complex (i.e., a formed complex) that includes the polymer scaffold 309, the fusion molecule 302 and the target analyte 305.

However, detection of a target analyte in a bulk sample may still be difficult due to the presence of background molecules which provide a variety of current impedance signatures which may be hard to distinguish of the formed complex in a nanopore. Therefore, the attachment of labels to the polymer scaffold to provide a unique electrical signal that is part of the event used to detect the analyte-fusion molecule complex may be used to identify the polymer scaffold as causing the event. Therefore, electrical signals that are part of the event caused by the polymer scaffold translocating through the nanopore can be distinguished from background molecules in an unfiltered bulk sample, such as whole blood. This innovative method of detection and polymer scaffold composition provides a quick and effective means of identifying target analytes in an unfiltered sample, while reducing error of false positive or false negatives in detection.

The formed complex can be detected using a device 308 that includes a nanopore (or simply, pore) 307, and a sensor. The pore 307 is a nano-scale or micro-scale opening in a structure separating two volumes. The sensor is configured to identify objects passing through the pore 307. For example, in some embodiments, the sensor identifies objects passing through the pore 307 by detecting a change in a measurable parameter, wherein the change is indicative of an object passing through the pore 307. This device is referred throughout as a “nanopore device.” In some embodiments, the nanopore device 308 includes electrodes connected to power sources, for moving the polymer scaffold 309 from one volume to another, across the pore 307. As the polymer scaffold 309 can be charged or be modified to contain charges. By generating a potential or voltage across the pore 307 the movement of the polymer scaffold 309 is facilitated and controlled. In certain embodiments, the sensor comprises a pair of electrodes, which are configured both as a sensor to detect the passage of objects through the nanopore by reading current, and to provide a voltage, across the pore 307. In certain embodiments, a voltage-clamp or a patch-clamp is used to simultaneously supply a voltage across the pore and measure the current through the pore.

When a sample that includes the formed complex is in the nanopore device 308, the nanopore device 308 can be configured to pass the formed complex including the polymer scaffold 309 through the pore 307. When the fusion molecule binding domain 301 is within the pore or adjacent to the pore 307, the binding status of the fusion molecule binding domain 301 can be detected by the sensor through current impedance or equivalent electrical signature.

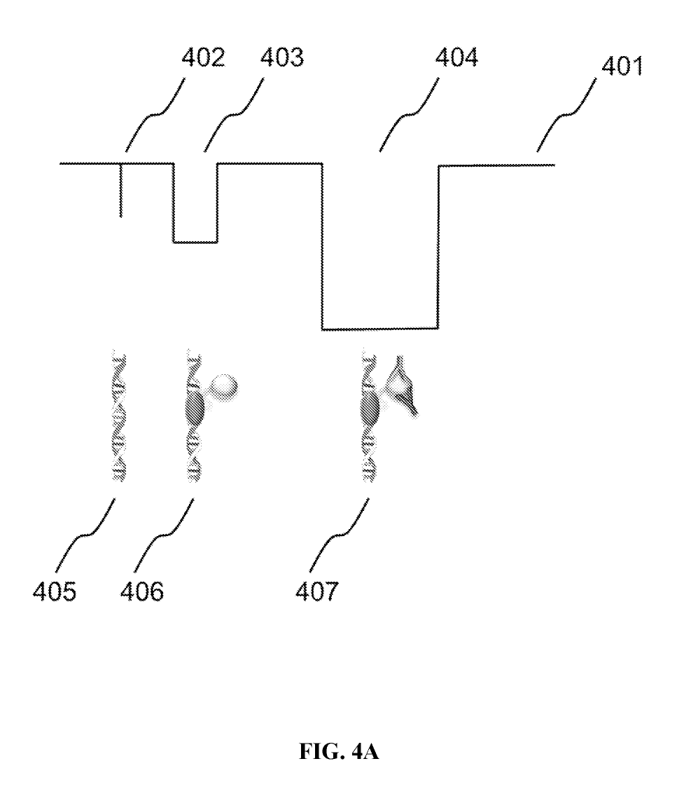

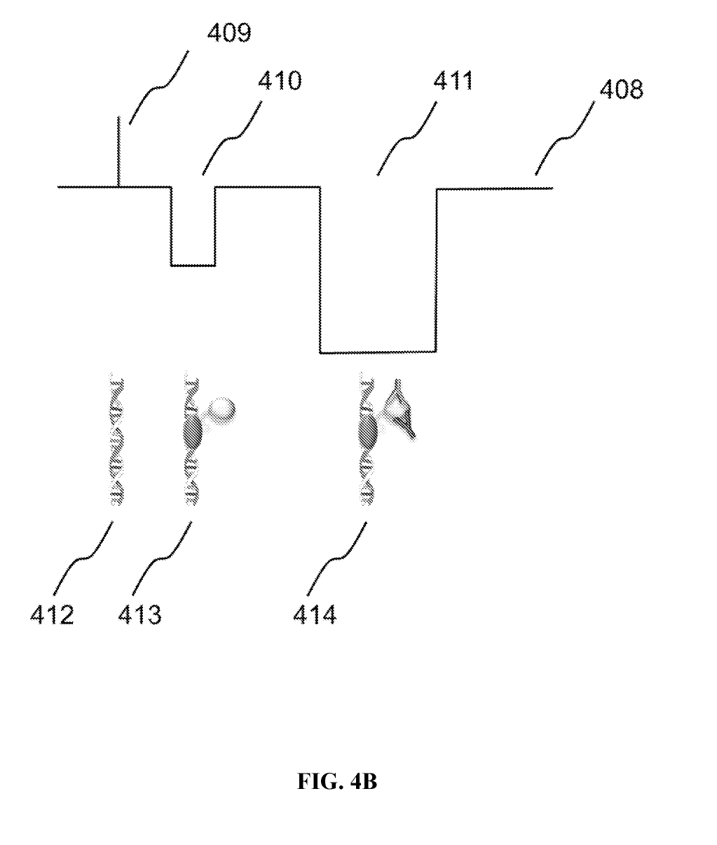

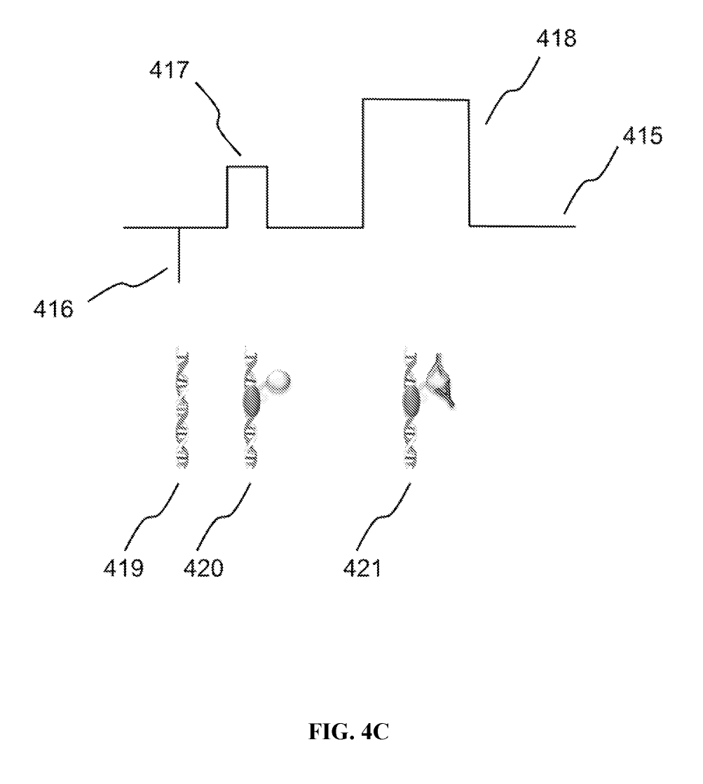

The “binding status” of a fusion molecule binding domain, as used herein, refers to whether the fusion molecule binding domain is bound to a fusion molecule with a corresponding scaffold binding domain, and whether the fusion molecule is also bound to a target analyte. Essentially, the binding status can be one of three potential statuses: (i) the fusion molecule binding domain is free and not bound to a fusion molecule (see 405 in FIG. 4); (ii) the fusion molecule binding domain is bound to a fusion molecule that does not bind to a target analyte (see 406 in FIG. 4); or (iii) the fusion molecule binding domain is bound to a fusion molecule that is bound to a target analyte (see 407 in FIG. 4).

Detection of the binding status of a fusion molecule binding domain can be carried out by various methods. In one aspect, by virtue of the different sizes of molecules bound to the binding domain at each status, when the binding domain passes through the pore, the electrical signal will correlate to the binding status. In one aspect, as shown in FIG. 4A, with a positive voltage applied and KCl concentrations greater than 0.4 M in the experiment buffer, the measured current signals 401, when 405, 406, and 407 pass through the pore, are signals 402, 403, and 404, respectively. All three event types are subjected to current attenuation when KCl concentrations are greater than 0.4 M, causing a reduction in the positive current flow. The three signals 402, 403, and 404 can be differentiated from one another by the amount of the current shift (height) and/or the duration of the current shift (width), or by any other feature in the signal that differentiates the three event types. It can also be that 404 is commonly different than 402 and 403, but that 402 and 403 are not commonly different from each other, in which case, robust detection of the biomarker bound to the passing molecule can still be accomplished. In another aspect, as shown in FIG. 4B, with a positive voltage applied and KCl concentrations less than 0.4M in the experiment buffer, the measured current signals 408, when 412, 413, and 414 pass through the pore, are signals 409, 410, and 411, respectively. Passage of dsDNA alone causes current enhancement events (409) at KCl concentrations less than 0.4 M. This was shown in the published research by Smeets, Ralph M M, et al. “Salt dependence of ion transport and DNA translocation through solid-state nanopores.” Nano Letters 6.1 (2006): 89-95. Hence, the signal 409 can be differentiated from 410 and 411 by the event amplitude direction (polarity) relative to the open channel baseline current level (408), in addition to the three signals commonly having different amounts of the current shift (height) and/or the duration of the current shift (width), or by any other feature in the signal that differentiates the three event types. In another aspect, as shown in FIG. 4C, with a negative voltage applied and KCl concentrations less than 0.4 M in the experiment buffer, the negative measured current signals 415, when 419, 420, and 421 pass through the pore, are signals 416, 417, and 418, respectively. Compared to signals 409, 410, and 411 with a positive voltage, the signals 416, 417, and 418 have the opposite polarity since the applied voltage has the opposite (negative) polarity. In all aspects of the FIG. 4 embodiments, the sensor comprises electrodes, which are connected to power sources and can detect the current. Either one or both of the electrodes, therefore, serve as a “sensor.” In this embodiment, a voltage-clamp or a patch-clamp is used to simultaneously supply a voltage across the pore and measure the current through the pore.

In some aspects, an agent 306 as shown in FIG. 3 is added to the complex to aid detection. This agent is capable of binding to the target analyte or the ligand/target analyte complex. In one aspect, the agent includes a charge, either negative or positive, to facilitate detection. In another aspect, the agent adds size to facilitate detection. In another aspect, the agent includes a detectable label, such as a fluorophore.

In this context, an identification of status (iii) indicates that a polymer scaffold-fusion molecule-target analyte complex has formed. In other words, the target analyte is detected.

### Larger Analyte Detection

The present disclosure also provides, in some aspects, methods and systems for detecting, quantitating, and measuring target analytes such as proteins, protein aggregates, oligomers, or protein/DNA complexes, or cells and microorganisms, including viruses, bacteria, and cellular aggregates.

In some aspects, the pore within the structure that separates the device into two volumes has a size that allows larger analytes, such as viruses, bacteria, cells, or cellular aggregates, to pass through. A fusion molecule having a ligand with an analyte binding moiety capable of binding to a larger target analyte to be detected or quantitated can be included in the solution in the nanopore device such that the ligand can bind to the unique target analyte and the polymer scaffold through a fusion molecule, generating a formed complex with the target analyte. Many such analytes have unique markers on their surfaces that can be specifically recognized by an analyte binding moiety on the ligand. For instance, tumor cells can have tumor antigens expressed on the cell surface, and bacterial cells can have endotoxins attached on the cell membrane.

When the formed complex in a solution loaded into the nanopore device is moved along with the polymer scaffold to pass through the pore, the binding status of the fusion molecule to the target analyte within or adjacent to the pore can be detected such that the analytes bound to the ligands can be identified using methods similar to the molecular detection methods described elsewhere in the disclosure.

### Multiplexing

In some aspects, rather than including multiple fusion molecule binding domains of the same kind as described above, a polymer scaffold can include multiple types of fusion molecule binding domains, each having different corresponding binding domains. In such embodiments, a sample can include multiple types of fusion molecules, each type including one of the different corresponding binding domains and a ligand for a different target analyte.

An additional method of multiplexing includes assaying a collection of different scaffold molecules during a test, with each different scaffold associating with different fusion molecule(s). To determine what target analytes are in solution, scaffolds of the same type are labeled such that the sensor can identify what fusion molecule will bind to that particular scaffold. This can be accomplished, for example, by barcoding each type of scaffold with polyethylene glycol molecules of varying lengths or sizes.

With such a setting, a single polymer scaffold can be used to detect multiple types of target analytes, including target molecules, target microorganisms (e.g. bacterium or virus), or target cells (e.g. circulating tumor cells). FIG. 5 illustrates such a method. Here, a double-stranded DNA 503 is used as the polymer scaffold, the double-stranded DNA 503 including multiple fusion molecule binding domains: two copies of a first fusion molecule binding domain 504, two copies of a second fusion molecule binding domain 505, and one copy of a third fusion molecule binding domain 506.

In some embodiments, the multiplexing polymer scaffold also comprises at least one label bound to a label binding domain on the polymer scaffold. In this manner, an electrical signal provided by the label—polymer scaffold complex can identify the polymer scaffold in an event. Thus, individual electrical signals attributed to polymer scaffold —fusion molecule complexes can be more easily detected and analyzed to determine the presence of an analyte based on the electrical signal.

When the DNA passes through a nanopore device 507 that has two coaxial pores, the binding status of each of the fusion molecule binding domains is detected. Each fusion molecule binding domain 504 bind to a corresponding target analyte. In one embodiment, electrical signals arising from unique bound fusion molecules 504 are distinguishable from other fusion molecule analyte complexes, and thus can be used for multiplexed detection of analytes on a single scaffold. In certain embodiments, the electrical signals from fusion molecules can be read in sequence and their identity determined by their relative position. Whether or not the fusion molecule is bound to an analyte can be detected as the DNA passes through a nanopore device.

This way, with a single polymer scaffold and a single nanopore device, the present technology can simultaneously detect multiple different target analytes. Further, by determining how many copies of fusion molecule binding domains are bound to the target analytes, and by tuning conditions that impact the bindings, the system can obtain more detailed binding dynamic information.

### Polymer Scaffold

A polymer scaffold suitable for use in the present technology is a scaffold that can be loaded into a nanopore device and passed through the pore from one end to the other.

Non-limiting examples of polymer scaffolds include nucleic acids, such as deoxyribonucleic acid (DNA), ribonucleic acid (RNA), or peptide nucleic acid (PNA), dendrimers, and linearized proteins or peptides. In some aspects, the DNA or RNA can be single-stranded or double-stranded, or can be a DNA/RNA hybrid molecule. In some aspects, the polymer scaffold can be dsDNA that is melted and hybridized to probes, resulting in a dsDNA, partially ssDNA/dsDNA, or a dsDNA in which one strand (e.g., sense or anti-sense) comprises one or more shorter sequences that hybridize, but are not ligated together.

In certain embodiments, double stranded DNA is used as a polymer scaffold. There are several advantages of dsDNA over ssDNA as a polymer scaffold. In general, non-specific interactions and unpredictable secondary structure formation are more prevalent in ssDNA, making dsDNA more suitable for generating reproducible electrical signals in a nanopore device. Also, ssDNA elastic response is more complex than dsDNA, and the properties of ssDNA are less well known than for dsDNA. Therefore, many embodiments of the invention are engineered to encompass dsDNA as a polymer scaffold, including several of the labels and fusion molecules used herein.

In one aspect, the polymer scaffold is synthetic or chemically modified. Chemical modification can help to stabilize the polymer scaffold, add charges to the polymer scaffold to increase mobility, maintain linearity, or add or modify the binding specificity, or add chemically reactive sites to which labels or ligands can be tethered. In some aspects, the chemical modification is acetylation, methylation, summolation, oxidation, phosphorylation, glycosylation, thiolation, addition of azides, or alkynes or activated alkynes (DBCO-alkyne), or the addition of biotin.

In some aspects, the polymer scaffold is electrically charged. DNA, RNA, PNA and proteins are typically charged under physiological conditions. Such polymer scaffolds can be further modified to increase or decrease the carried charge. Other polymer scaffolds can be modified to introduce charges. Charges on the polymer scaffold can be useful for driving the polymer scaffold to pass through the pore of a nanopore device. For instance, a charged polymer scaffold can move across the pore by virtue of an application of voltage across the pore.

In some aspects, when charges are introduced to the polymer scaffold, the charges can be added at the ends of the polymer scaffold. In some aspects, the charges are spread over the polymer scaffold.

In an embodiment, each unit of the charged polymer scaffold is charged at the pH selected. In another embodiment, the charged polymer scaffold includes sufficient charged units to be pulled into and through the pore by electrostatic forces. For example, a peptide containing sufficient entities can be charged at a selected pH (lysine, aspartic acid, glutamic acid, etc.) so as to be used in the devices and methods described herein. Likewise, a co-polymer comprising methacrylic acid and ethylene is a charged polymer for the purposes of this invention if there is sufficient charged carboxylate groups of the methacrylic acid residue to be used in the devices and methods described herein. In an embodiment, the charged polymer scaffold includes one or more charged units at or close to one terminus of the polymer scaffold. In another embodiment, the charged polymer scaffold includes one or more charged units at or close to both termini of the polymer scaffold. One co-polymer example is a DNA wrapped around protein (e.g. DNA/nucleosome). Another example of a co-polymer is a linearized protein conjugated to DNA at the N- and C-terminus. Another example of a co-polymer is a DNA bound to a protein that provides rigidity (e.g. RecA protein) or charge.

The much-improved polymer scaffold decoding technology as provided above makes it practical to use polymer scaffolds for data storage, which is also within the scope of the present disclosure. For instance, using the codes (A, B, C and D) illustrated in FIG. 1, a polynucleotide can be synthesized, including label binding domains for labels A, B, C and/or D. Such label binding domains can be detected by a nanopore device as presently described, through binding to the corresponding labels. In other embodiments, A, B, C, and D themselves are labels, insofar as they generate detectably different signals when passing through the nanopore. Therefore, the composition and sequence of the polynucleotide in terms of the label binding domains constitute an information storage, and A, B, C and D represent the code of the storage.

Using polymer scaffolds for data storage, it is contemplated there are many advantages over conventional computer memory technologies, which are bound to a binary number system (0's and 1's) due to the fact that data is stored using electronic gates, which can be in only one of two states (on and off) for each location in the memory unit. The presently disclosed technology can accommodate an arbitrarily large number of different labels in the same location, as described below; hence the variation in each code is much greater than the 1's and 0's of a binary system. Accordingly, the data capacity of each unit is much greater. Further, decoding of the data can be faster, given that nanopore-based label detection can be multiplexed in parallel, where hundreds, thousands or each millions of nanopores on a single membrane.

Thus, in certain embodiments, the present disclosure provides a polymer scaffold-based data storage device and methods for encoding and decoding the data in the device. The polymer scaffold can be synthesized to include label binding domains which serve as codes for the data. Before or during reading the data, the polymer scaffold is placed in contact with the labels under conditions where the labels can bind to the label binding domains. The polymer scaffold that is bound to the labels can then be subjected to label detection by a nanopore device. Finally, the detected labels can be compiled to represent the data.

In some aspects, the labels can be permanently linked to the polymer scaffold. For example this can be done by cross-linking the labels to the scaffold using formaldehyde if the labels are proteins. In another aspect, chemical coupling can be used to link the label to the scaffold.

### Probe Binding Domains on the Scaffold

For nucleic acids and polypeptides such as the polymer scaffold, a probe (e.g., a label or fusion molecule) binding domain can be a nucleotide or peptide sequence that is recognizable by a scaffold binding domain on the probe. In some embodiments, the probe binding domain is a peptide sequence forming a functional portion of a protein, although the binding domain does not have to be a protein. For nucleic acids, for instance, there are proteins that specifically recognize and bind to sequences (motifs) such as promoters, enhancers, thymine-thymine dimers, and certain secondary structures such as bent nucleotide and sequences with single-strand breakage.

In some aspects, the probe binding domain includes a chemical modification that causes or facilitates recognition and binding by a polymer scaffold binding domain. For example, methylated DNA sequences can be recognized by transcription factors, DNA methyltransferases or methylation repair enzymes. In other embodiments, biotin may be incorporated into, and recognized by, avidin family members. In such embodiments, biotin forms the probe binding domain and avidin or an avidin family member is the polymer scaffold binding domain on the probe. Due to their binding complementarity, probe binding domains and polymer scaffold domains may be reversed so that the probe binding domain becomes the polymer scaffold binding domain, and vice versa.

Molecules, in particular proteins, that are capable of specifically recognizing nucleotide binding motifs are known in the art. For instance, protein domains such as helix-turn-helix, a zinc finger, a leucine zipper, a winged helix, a winged helix turn helix, a helix-loop-helix and an HMG-box, are known to be able to bind to nucleotide sequences.

In some aspects, the probe binding domains can be locked nucleic acids (LNAs), bridged nucleic acids (BNA), Protein Nucleic Acids of all types (e.g. bisPNAs, gamma-PNAs), transcription activator-like effector nucleases (TALENs), clustered regularly interspaced short palindromic repeats (CRISPRs), or aptamers (e.g., DNA, RNA, protein, or combinations thereof). In some aspects the probe forms a triplex complex with the scaffold, in others, it forms a duplex complex with the scaffold.

In some aspects, the probe binding domains are one or more of DNA binding proteins (e.g., zinc finger proteins), antibody fragments (Fab), chemically synthesized binders (e.g., PNA, LNA, TALENS, or CRISPR), or a chemical modification (i.e., reactive moieties) in the synthetic polymer scaffold (e.g., thiolate, biotin, amines, carboxylates).

In some embodiments, the polymer scaffold includes a sequence of label binding domains which are used to encode information in the polymer scaffold. In other embodiments, the polymer scaffold also includes a fusion molecule binding domain for analyte detection, in combination with at least one label binding domain for scaffold identification. In some embodiments, the polymer scaffold can include a plurality of unique fusion molecule binding domains for multiplexed analyte detection on a single polymer scaffold.

In some embodiments, sequence specificity at the single-nucleotide level is critical to maximizing polymer scaffold binding and maintaining high fidelity of detection. Short duplex-forming probes (<50 nucleotide) can be used for precise sequence detection. In some embodiments, provided herein are duplex-forming ssDNA probes sensitive to single-nucleotide mismatches, yet robust enough to remain bound though high salt nanopore buffers. These probes can be bound to a detectable tag to form a label or can be bound to a target binding moiety to form a fusion molecule.

In some embodiments, the DNA binding probes are generated by selectively substituting DNA bases for conformation-locked bases. Specifically, we developed probes containing locked nucleic acid LNA™ (Exiqon Inc.), or, bridged nucleic acid BNA™ (Biosynthesis Inc.) bases. These substitutions enhance sequence detection specificity and increase probe binding affinity to DNA.

### Labels

In some embodiments, the label includes a protein that specifically recognizes and binds a specific label binding domain on the polymer scaffold. For nucleic acids and polypeptides as the polymer scaffold, a label binding domain can be a nucleotide or peptide sequence that is recognizable by a binding protein, which is typically a functional portion of a protein. For nucleic acids, for instance, there are proteins that specifically recognize and bind to sequences (motifs) such as promoters, enhancers, thymine-thymine dimers, and certain secondary structures such as bent nucleotide and sequences with single-strand breakage.

In some aspects, the label includes a chemical modification that causes or facilitates recognition and binding by a label binding domain. For example, methylated DNA sequences can be recognized by transcription factors, DNA methyltransferases or methylation repair enzymes.

Molecules, in particular proteins, that are capable of specifically recognizing nucleotide binding domains are known in the art. For instance, protein domains such as helix-turn-helix, a zinc finger, a leucine zipper, a winged helix, a winged helix turn helix, a helix-loop-helix and an HMG-box, are known to be able to bind to nucleotide sequences.

Any molecule that specifically binds to a label binding domain on a polymer scaffold, which can be characterized by the sequence or structure, can be a label. Examples of label molecules include a peptide, a nucleic acid, TALENS, CRISPR, LNA, a PNA (protein nucleic acid), bis-PNA, gamma-PNA, a PNA-conjugate that increases size or charge of PNA, or any other PNA derived polymer, and a chemical compound, e.g. polyethylene glycol of various lengths.

A PNA is a synthetic form of nucleic acid which lacks a net electrical charge along its protein-like backbone. PNAs have found a number of applications in vitro, as well as in vivo to tag specific genomic sequences. In one aspect, at least one label is a bis-PNA. A bis-PNA molecule is made up of two PNA oligomers connected by a flexible linker. A few lysine residues are often added at their termini to improve association kinetics to dsDNA. It can spontaneously target dsDNA molecules with high affinity and sequence-specificity, relying on the simultaneous formation of Watson-Crick and Hoogsteen base-pairs. In other embodiments, the PNA can have certain modifications, such as in pseudo-complementary PNA (i.e., pcPNA) and gamma-PNA (i.e., γ-PNA). The synthesis of PNAs are well known in the art.

Generally, a bis-PNA is comprised of homopyrimidines or homopurines, and its binding of dsDNA generally requires a PNA/DNA triplex formation. This essentially limits the target regions for hybridization on the dsDNA to homopurine homopyrimidine stretches. In order to avoid the sequence limitations associated with PNAs such as bis-PNAs, so as to be able to target essentially any mixed DNA sequence, other modified PNA labels can be used (e.g. gamma PNA), or general nucleic acid probes can be used, e.g. LNA, BNA, DNA, RNA. In some aspects the probe forms a triplex complex with the scaffold, in others, it forms a duplex complex with the scaffold.

In some aspects, the at least one label is a γ-PNA. γ-PNA has a simple modification in a peptide-like backbone, specifically at the γ-position of the N-(2-aminoethyl)glycine backbone, thus generating a chiral center (Rapireddy S., et al., 2007. J. Am. Chem. Soc., 129:15596-600; He G, et al., 2009, J. Am. Chem. Soc., 131:12088-90; Chema V, et al., 2008, Chembiochem 9:2388-91; Dragulescu-Andrasi, A., et al., 2006, J. Am. Chem. Soc., 128:10258-10267). Unlike bis-PNA, γ-PNA can bind to dsDNA without sequence limitation, leaving one of the two DNA strands accessible for further hybridization.

In some aspects, the function of the label is to hybridize to the polymer scaffold by complement base pairing to form a stable complex. That complex has sufficiently large cross-section surface area to produce a detectable change or contrast in signal amplitude over that of the background, which is the mean or average signal amplitude corresponding to sections of non-label-bound target-bearing polymer scaffold.

The stability of the complex is important in order for it to be detected by a nanopore device. The complex's stability must be maintained throughout the period that the target-bearing polymer scaffold is being translocated through the nanopore. If the complex is weak, or unstable, the complex can fall apart and will not be detected as the target-bearing polymer scaffold threads through the nanopores. If the binding interaction between probe and scaffold is desired to be stronger, the probe can be transiently or reversibly cross-linked to the scaffold, e.g. using UV photo-crosslinking or reversible chemical crosslinking using formaldehyde.

In some aspects, the labels can be permanently linked to the polymer scaffold. For example this can be done by cross-linking the labels to the scaffold using formaldehyde if the labels are proteins. In another aspect, chemical coupling can be used to link the label to the scaffold.

The size of the complex including the polymer scaffold and the label has to have sufficient properties, e.g., size, hydrophobicity and charge, to generate a detectable electrical signal when the complex threads through the nanopore which deviates from the background noise. In some embodiments, this may be performed by adding a detectable tag to a label comprising a polymer scaffold binding domain. This detectable tag may be modified by its width, length, size, or charge to affect the electrical signal generated by measuring current impedance as the label comprising a detectable tag and bound to a polymer scaffold translocates through the nanopore. An example of the use of labels bound to detectable tags is shown in FIG. 1, where labels A, B, C, and D each have a unique detectable tag to generate a distinguishable electrical signal to allow identifications of the labels, and therefore the label binding sites, as the polymer scaffold translocates through the nanopore.

FIG. 2 shows a PNA label that has been modified by addition of a detectable tag so as to increase its size, and therefore facilitate detection. Specifically, this label, which binds to the target DNA sequence by complementary base pairing between the bases on the PNA molecule (204) and the bases in the target DNA, has cysteine residues incorporated into the backbone (201 dotted line box), which provide a free thiol chemical handle for conjugation to a detectable tag. Here, the cysteine is bound to a peptide (203) through a maleimide linker (202 dotted line box). The peptide acts as a detectable tag, providing a means to better detect whether the label is bound to its target sequence upon translocation through the nanopore, since the label/peptide gives an increase to the label size. This greater size results in a greater change in current flow through the pore, or current impedance, compared to an unlabeled PNA.

In a particular embodiment, a label is a PNA conjugated to a detectable tag, in which the PNA portion specifically recognizes a nucleotide sequence, and the detectable tag increases the size/shape/charge differences between different PNA conjugates.

In some aspects, to increase the contrast in the change between the label-bound polymer scaffold complex and other molecules present in the sample, modification can be made to the pseudo-peptide backbone to change the overall charge of the label (e.g., PNA) to increase the contrast. Selection of more charged amino acids instead of non-polar amino acids can serve to increase the charge of PNA. In addition, smaller detectable tags, such as molecules, proteins, peptides, or polymers (e.g., PEG) can be conjugated to the pseudo-peptide backbone to enhance the bulk or cross-sectional surface area of the label and target-bearing polymer scaffold complex. Enhanced bulk serves to enhance the signal amplitude contrast so that any differential signal resulting from the increased bulk can be easily detected. Small molecules, such as organic molecules, proteins, or peptides, can be conjugated to the pseudo-peptide backbone. These molecules include, but are not limited to, nanometer-sized gold particles (e.g. 3 nm), quantum dots, polyethylene glycol (PEG), polyvinyl pyrrolidone, polyvinyl alcohol, polyamino acids, divinylether maleic anhydride, N-(2-Hydroxypropyl)-methacrylamide, dextran, dextran derivatives including dextran sulfate, polypropylene glycol, polyoxyethylated polyol, heparin, heparin fragments, polysaccharides, cellulose and trypsin inhibitors. Methods of conjugation of molecules are well known in the art, e.g. in U.S. Pat. Nos. 5,180,816, 6,423,685, 6,706,252, 6,884,780, and 7,022,673, which are hereby incorporated by reference in their entirety. Examples of some conjugating agents include, but are not limited to, ethylenediaminetetraacetic acid (EDTA), diethylenetriaminopentaacetic acid (DTPA), ethyleneglycol-0,0′-bis(2-aminoethyl)-N,N,N′,N′-tetraacetic acid (EGTA), N,N′-bis(hydroxybenzyl)ethylenediamine-N,N′-diacetic acid (HBED), triethylenetetraminehexaacetic acid (TTHA), 1,4,7,10-tetra-azacyclododecane-N,N′,N″,N′″-tetraacetic acid (DOTA), 1,4,7,10-tetraazacyclotridecane-1,4,7,10-tetraacetic acid (TITRA), 1,4,8,11-tetraazacyclotetradecane-N,N′,N″,N′″-tetraacetic acid (TETA), and 1,4,8,11-tetraazacyclotetradecane (TETRA).

In some aspects, the label needs not entirely hybridize to the target-bearing polymer scaffold. It can be sufficient that a portion of the label binds to the target-bearing polymer scaffold. In some aspects, at least 50% of the label binds to the target-bearing polymer scaffold. In some aspects, at least 5%, at least 10%, at least 15%, at least 25%, at least 30%, at least 35%, at least 40%, or at least 45% of the label binds to the target-bearing polymer scaffold.

Different reactive moieties may be incorporated into the labels to provide chemical handles to which labels may be conjugated to serve as detectable tags. Examples of reactive moieties, which may be included in the scaffold itself or probe (such as PNA), include, but are not limited to, primary amines, carboxylic acids, ketones, amides, aldehydes, boronic acids, hydrazones, thiols, maleimides, alcohols, and hydroxyl groups.

A common method for incorporating the chemical handles is to include a specific amino acid into the backbone of the label. Examples include, but are not limited to, cysteines (provide thiolates), lysines (provide free amines), threonine (provides hydroxyl), glutamate and aspartate (provides carboxylic acids). Examples of this are detectable tags that add size, charge, or fluorescence to the label.

Different types of labels can be added using the reactive moieties. These include labels that: 1) increase the size of the label, e.g. biotin/streptavidin, peptide, nucleic acid; 2) change the charge of the label, e.g. a charged peptide (6×HIS), or protein (charybdotoxin); and 3) change or add fluorescence to the label, e.g. common fluorophores, FITC, Rhodamine, Cy3, Cy5.

The labels may be detected by methods known in the art as an alternative to the use of current impedance. Useful labels include, e.g., fluorescent dyes (e.g., Cy5®, Cy3®, FITC, rhodamine, lanthamide phosphors, Texas red), 32P, 35S, 3H, 14C, 125I, 131I, electron-dense reagents (e.g., gold), enzymes as commonly used in an ELISA (e.g., horseradish peroxidase, beta-galactosidase, luciferase, alkaline phosphatase), colorimetric labels (e.g., colloidal gold), magnetic labels (e.g., Dynabeads™), biotin, dioxigenin, or haptens and proteins for which antisera or monoclonal antibodies are available. Other labels include labels or oligonucleotides capable of forming a complex with the corresponding receptor or oligonucleotide complement, respectively. The label can be directly incorporated into the nucleic acid to be detected, or it can be bound to a label (e.g., an oligonucleotide) or antibody that hybridizes or binds to the nucleic acid to be detected.

In some aspects, the label is a fluorophore. The term “fluorophore” as used herein refers to a molecule that absorbs light at a particular wavelength (excitation frequency) and subsequently emits light of a longer wavelength (emission frequency). The term “donor fluorophore” as used herein means a fluorophore that, when in close proximity to a quencher moiety, donates or transfers emission energy to the quencher. As a result of donating energy to the quencher moiety, the donor fluorophore will itself emit less light at a particular emission frequency that it would have in the absence of a closely positioned quencher moiety.

Suitable fluorescent moieties include the following fluorophores known in the art: 4-acetamido-4′-isothiocyanatostilbene-2,2′disulfonic acid acridine and derivatives: acridine, acridine isothiocyanate, Alexa Fluor® 350, Alexa Fluor® 488, Alexa Fluor® 546, Alexa Fluor® 555, Alexa Fluor® 568, Alexa Fluor® 594, Alexa Fluor® 647 (Molecular Probes); 5-(2′-aminoethyl)aminonaphthalene-1-sulfonic acid (EDANS), 4-amino-N-(3-vinyl sulfonyl) phenyl]naphthalimide-3,5 disulfonate (Lucifer Yellow VS), N-(4-anilino-1-naphthyl) maleimide, anthranilamide, Black Hole Quencher™ (BHQ™) dyes (biosearch Technologies), BODIPY® R-6G, BOPIPY® 530/550, BODIPY® FL Brilliant Yellow; coumarin and derivatives: coumarin, 7-amino-4-methylcoumarin (AMC, Coumarin 120); 7-amino-4-trifluoromethylcouluarin (Coumarin 151), Cy2®, Cy3®, Cy3.5®, Cy5®, Cy5.5®; Cyanosine 4′,6-diaminidino-2-phenylindole (DAPI) 5′,5″-dibromopyrogallol sulfonephthalein (Bromopyrogallol Red), 7-diethylamino-3-(4′-isothiocyanatophenyl)-4-methylcoumarin diethylenetriamine pentaacetate, 4,4′-diisothiocyanatodihydro-stilbene-2,2′-disulfonic acid, 4,4′-diisothiocyanatostilbene-2,2′-disulfonic acid 5 [dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC), Eclipse™ (Epoch Biosciences Inc.); eosin and derivatives: eosin, eosin isothiocyanate; erythrosin and derivatives: erythrosin B, erythrosin isothiocyanate, ethidium fluorescein and derivatives: 5-carboxyfluorescein (FAM), 5-(4,6-dichlorotriazin-2-yl)aminofluorescein (DTAF), 2′,7′-dimethoxy-4′5′-dichloro-6-carboxyfluorescein (JOE), fluorescein, fluorescein isothiocyanate (FITC), hexachloro-6-carboxyfluorescein (HEX), QFITC (XRITC), tetrachlorofluorescein (TET), fluorescamine, IR144, IR1446, Malachite Green isothiocyanate, 4-methylumbelliferone, ortho cresolphthalein, nitrotyrosine, pararosaniline, Phenol Red, B-phycoerythrin, R-phycoerythrin, o-phthaldialdehyde, Oregon Green®, propidium iodide; pyrene and derivatives: pyrene, pyrene butyrate, succinimidyl 1-pyrene butyrate, QSY® 7, QSY® 9, QSY® 21, QSY® 35 (Molecular Probes), Reactive Red 4 (Cibacron® Brilliant Red 3B-A); rhodamine and derivatives: 6-carboxy-X-rhodamine (ROX), 6-carboxyrhodamine (R6G), lissamine rhodamine B sulfonyl chloride, rhodamine (Rhod), rhodamine B, rhodamine 123, rhodamine green, rhodamine X isothiocyanate, sulforhodamine B, sulforhodamine 101, sulfonyl chloride derivative of sulforhodamine 101 (Texas Red), N,N,N′,N′-tetramethyl-6-carboxyrhodamine (TAMRA), tetramethyl rhodamine, tetramethyl rhodamine isothiocyanate (TRITC), riboflavin, rosolic acid, terbium chelate derivatives.

Other fluorescent nucleotide analogs can be used, see, e.g., Jameson et al., 278 Meth. Enzymol. 363-390 (1997); Zhu et al., 22 Nucl. Acids Res. 3418-3422 (1994). U.S. Pat. Nos. 5,652,099 and 6,268,132 also describe nucleoside analogs for incorporation into nucleic acids, e.g., DNA and/or RNA, or oligonucleotides, via either enzymatic or chemical synthesis to produce fluorescent oligonucleotides. U.S. Pat. No. 5,135,717 describes phthalocyanine and tetrabenztriazaporphyrin reagents for use as fluorescent labels.

The labels can be incorporated into, associated with, or conjugated to, a nucleic acid. Labels can be bound by spacer arms of various lengths to reduce potential steric hindrance or impact on other useful or desired properties. See, e.g., Mansfield, 9 Mol. Cell. Probes 145-156 (1995).

The labels can be incorporated into nucleic acids by covalent or non-covalent means, e.g., by transcription, such as by random-primer labeling using Klenow polymerase, or nick translation, or amplification, or equivalent, as is known in the art. For example, a nucleotide base is conjugated to a detectable moiety, such as a fluorescent dye, e.g., Cy3® or Cy5®, and then incorporated into genomic nucleic acids during nucleic acid synthesis or amplification. Nucleic acids can thereby be labeled when synthesized using Cy3®- or Cy5®-dCTP conjugates mixed with unlabeled dCTP.

Nucleic acid labels can be modified by using PCR or nick translation in the presence of labeled precursor nucleotides, for example. Modified nucleotides synthesized by coupling allylamine-dUTP to the succinimidyl-ester derivatives of the fluorescent dyes or haptens (e.g., biotin or digoxigenin) can be used; this method allows custom preparation of most common fluorescent nucleotides, see, e.g., Henegariu et al., Nat. Biotechnol. 18:345-348 (2000).

Nucleic acid labels may be labeled by non-covalent means known in the art. For example, Kreatech Biotechnology's Universal Linkage System® (ULS®) provides a non-enzymatic labeling technology, wherein a platinum group forms a coordinative bond with DNA, RNA or nucleotides by binding to the N7 position of guanosine. This technology may also be used to label proteins by binding to nitrogen and sulfur containing side chains of amino acids. See, e.g., U.S. Pat. Nos. 5,580,990; 5,714,327; and 5,985,566; and European Patent No. 0539466.

### Fusion Molecule

A “fusion molecule” is intended to mean a molecule or complex that contains two functional regions, a polymer scaffold binding domain and a ligand comprising an analyte binding moiety. The polymer scaffold binding domain is capable of binding to a fusion molecule binding domain on a polymer scaffold, and the ligand is capable of binding to a target analyte.

In some aspects, the fusion molecule is prepared by linking the two regions with a bond or force. Such a bond and force can be, for instance, a covalent bond, a hydrogen bond, an ionic bond, a metallic bond, van der Walls force, hydrophobic interaction, or planar stacking interaction. The fusion molecule can also be a contiguous stretch of nucelotides, wherein one portion of the contiguous stretch of nucleotides (i.e., a polynucleotide) acts as the scaffold binding domain, and another portion of the stretch of contiguous nucleotides is adapted to bind an analyte (i.e., comprises an analyte binding domain), in particular if the analyte is nucleic acid.

In some aspects, the fusion molecule, such as a fusion protein, can be expressed as a single molecule from a recombinant coding nucleotide. In some aspects, the fusion molecule is a natural molecule having a polymer scaffold binding domain and a ligand suitable for use in the present technology.

Many options exist for connecting the polymer scaffold binding domain with the ligand to form the fusion molecule. For example, the components may be connected via chemical coupling through functionalized linkers such as free amine, carboxylate coupling, thiolate, hydrazide, or azide (click) chemistry or the polymer scaffold binding domain and the ligand may form one continuous transcript.

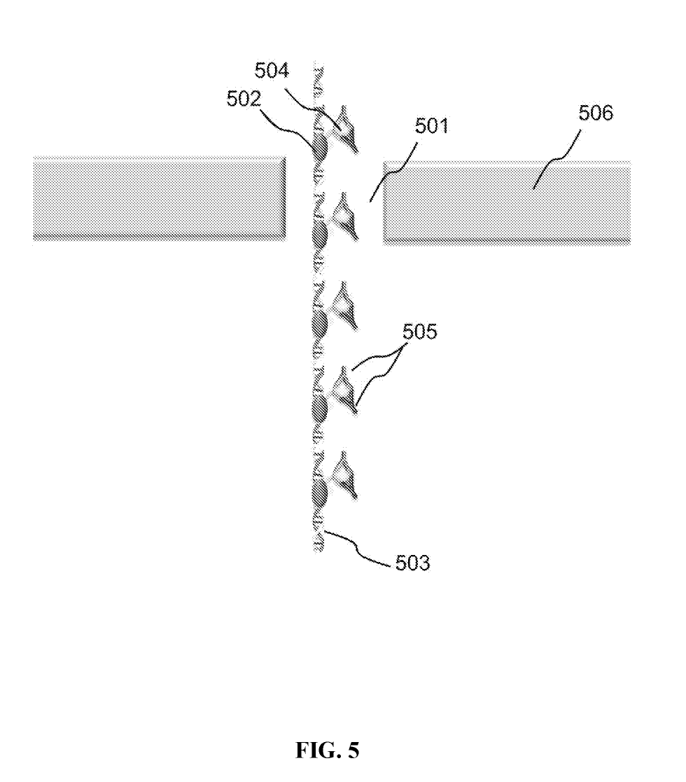

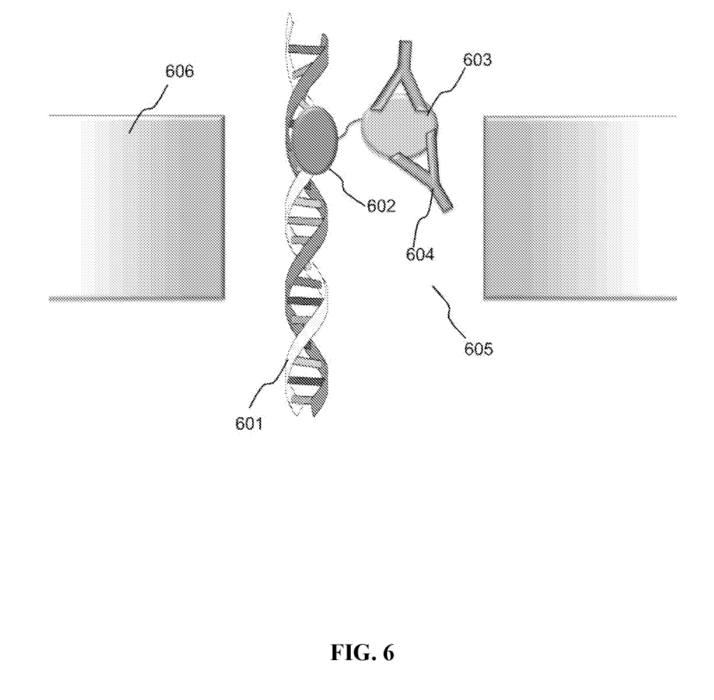

FIG. 6 illustrates a more specific embodiment of the system shown in FIG. 3.

In FIG. 6, the fusion molecule is a chimeric protein that includes a zinc finger protein or domain 602 and a human immunodeficiency virus (HIV) envelop protein 603. The zinc finger protein 602 has polymer scaffold binding domain that can bind to a suitable nucleotide sequence on the polymer scaffold, a double-stranded DNA 601; the HIV envelop protein 603 is a ligand with an analyte binding moiety that can bind to an anti-HIV antibody 604 which can be present in a biological sample (e.g., a blood sample from a patient) for detection.

When the double-stranded DNA 601 passes through a pore 605 of a nanopore device 606, the nanopore device 606 can detect whether a fusion molecule is bound to the DNA 601 and whether the bound fusion molecule binds to an anti-HIV antibody 604.

FIG. 3B shows a fusion molecule that has an antibody analyte capture domain fused to a Azide reactive group through a PEG linker.

### Target Analytes and Ligands

In the present technology, a target analyte is detected or quantitated by virtue of its binding to a ligand in a fusion molecule that also binds to a polymer scaffold. A target analyte and a corresponding binding ligand with an analyte binding moiety can recognize and bind each other. For a larger analyte, there can be surface molecules or markers suitable for a ligand to bind (therefore the marker and the ligand form a binding pair).

Examples of binding pairs that enable binding between a target analyte and a ligand, but are not limited to, antigen/antibody (or antibody fragment); hormone, neurotransmitter, cytokine, growth factor or cell recognition molecule/receptor; and ion or element/chelate agent or ion binding protein, such as a calmodulin. The binding pairs can also be single-stranded nucleic acids having complementary sequences, enzymes and substrates, members of protein complex that bind each other, enzymes and cofactors, enzymes and one or more of their inhibitors (allosteric or otherwise), nucleic acid/protein, or cells or proteins detectable by cysteine-constrained peptides.

In some embodiments, the ligand is a protein, protein scaffold, peptide, aptamer (DNA or protein), nucleic acid (DNA or RNA), antibody fragment (Fab), chemically synthesized molecule, chemically reactive functional group or any other suitable structure that forms a binding pair with a target analyte.

Therefore, any target analyte in need of detection or quantitation, such as proteins, peptides, nucleic acids, chemical compounds, ions, and elements, can find a corresponding binding ligand. For the majority of proteins and nucleic acids, an antibody or a complementary sequence, or an aptamer can be readily prepared.

Likewise, binding ligands (such as antibodies and aptamers) can be readily found or prepared for analytes such as protein complexes and protein aggregates, protein/nucleic acid complexes, fragmented or fully assembled viruses, bacteria, cells, and cellular aggregates.

### Nanopore Devices

A nanopore device, as provided, includes at least a pore that forms an opening in a structure separating an interior space of the device into two volumes, and at least a sensor configured to identify objects (for example, by detecting changes in parameters indicative of objects) passing through the pore. Nanopore devices used for the methods described herein are also disclosed in PCT Publication WO/2013/012881, incorporated by reference in entirety.

The pore(s) in the nanopore device are of a nano scale or micro scale. In one aspect, each pore has a size that allows a small or large molecule or microorganism to pass. In one aspect, each pore is at least about 1 nm in diameter. Alternatively, each pore is at least about 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 25 nm, 30 nm, 35 nm, 40 nm, 45 nm, 50 nm, 60 nm, 70 nm, 80 nm, 90 nm, or 100 nm in diameter.

In one aspect, the pore is no more than about 100 nm in diameter. Alternatively, the pore is no more than about 95 nm, 90 nm, 85 nm, 80 nm, 75 nm, 70 nm, 65 nm, 60 nm, 55 nm, 50 nm, 45 nm, 40 nm, 35 nm, 30 nm, 25 nm, 20 nm, 15 nm, or 10 nm in diameter.

In some aspects, each pore is at least about 100 nm, 200 nm, 500 nm, 1000 nm, 2000 nm, 3000 nm, 5000 nm, 10000 nm, 20000 nm, or 30000 nm in diameter. In one aspect, the pore is no more than about 100000 nm in diameter. Alternatively, the pore is no more than about 50000 nm, 40000 nm, 30000 nm, 20000 nm, 10000 nm, 9000 nm, 8000 nm, 7000 nm, 6000 nm, 5000 nm, 4000 nm, 3000 nm, 2000 nm, or 1000 nm in diameter.

In one aspect, the pore has a diameter that is between about 1 nm and about 100 nm, or alternatively between about 2 nm and about 80 nm, or between about 3 nm and about 70 nm, or between about 4 nm and about 60 nm, or between about 5 nm and about 50 nm, or between about 10 nm and about 40 nm, or between about 15 nm and about 30 nm.

In some aspects, the pore(s) in the nanopore device are of a larger scale for detecting large microorganisms or cells. In one aspect, each pore has a size that allows a large cell or microorganism to pass. In one aspect, each pore is at least about 100 nm in diameter. Alternatively, each pore is at least about 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, 1600 nm, 1700 nm, 1800 nm, 1900 nm, 2000 nm, 2500 nm, 3000 nm, 3500 nm, 4000 nm, 4500 nm, or 5000 nm in diameter.

In one aspect, the pore is no more than about 100,000 nm in diameter. Alternatively, the pore is no more than about 90,000 nm, 80,000 nm, 70,000 nm, 60,000 nm, 50,000 nm, 40,000 nm, 30,000 nm, 20,000 nm, 10,000 nm, 9000 nm, 8000 nm, 7000 nm, 6000 nm, 5000 nm, 4000 nm, 3000 nm, 2000 nm, or 1000 nm in diameter.

In one aspect, the pore has a diameter that is between about 100 nm and about 10000 nm, or alternatively between about 200 nm and about 9000 nm, or between about 300 nm and about 8000 nm, or between about 400 nm and about 7000 nm, or between about 500 nm and about 6000 nm, or between about 1000 nm and about 5000 nm, or between about 1500 nm and about 3000 nm.

In some aspects, the nanopore device further includes means to move a polymer scaffold across the pore and/or means to identify objects that pass through the pore. Further details are provided below, described in the context of a two-pore device.

Compared to a single-pore nanopore device, a two-pore device can be more easily configured to provide good control of speed and direction of the movement of the polymer scaffold across the pores.

In certain embodiments, the nanopore device includes a plurality of chambers, each chamber in communication with an adjacent chamber through at least one pore. Among these pores, two pores, namely a first pore and a second pore, are placed so as to allow at least a portion of a polymer scaffold to move out of the first pore and into the second pore. Further, the device includes a sensor capable of identifying the polymer scaffold during the movement. In one aspect, the identification entails identifying individual components of the polymer scaffold. In another aspect, the identification entails identifying fusion molecules and/or target analytes bound to the polymer scaffold. When a single sensor is employed, the single sensor may include two electrodes placed at both ends of a pore to measure an ionic current across the pore. In another embodiment, the single sensor comprises a component other than electrodes.

In one aspect, the device includes three chambers connected through two pores. Devices with more than three chambers can be readily designed to include one or more additional chambers on either side of a three-chamber device, or between any two of the three chambers. Likewise, more than two pores can be included in the device to connect the chambers.

In one aspect, there can be two or more pores between two adjacent chambers, to allow multiple polymer scaffolds to move from one chamber to the next simultaneously. Such a multi-pore design can enhance throughput of polymer scaffold analysis in the device.

In some aspects, the device further includes means to move a polymer scaffold from one chamber to another. In one aspect, the movement results in loading the polymer scaffold across both the first pore and the second pore at the same time. In another aspect, the means further enables the movement of the polymer scaffold, through both pores, in the same direction.

For instance, in a three-chamber two-pore device (a “two-pore” device), each of the chambers can contain an electrode for connecting to a power supply so that a separate voltage can be applied across each of the pores between the chambers.

In accordance with an embodiment of the present disclosure, provided is a device comprising an upper chamber, a middle chamber and a lower chamber, wherein the upper chamber is in communication with the middle chamber through a first pore, and the middle chamber is in communication with the lower chamber through a second pore. Such a device may have any of the dimensions or other characteristics previously disclosed in U.S. Publ. No. 2013-0233709, entitled Dual-Pore Device, which is herein incorporated by reference in its entirety.

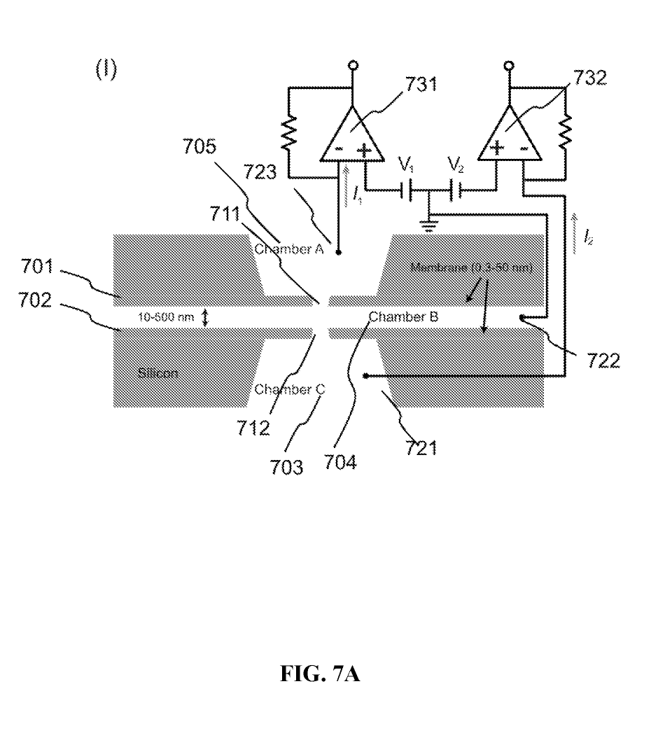

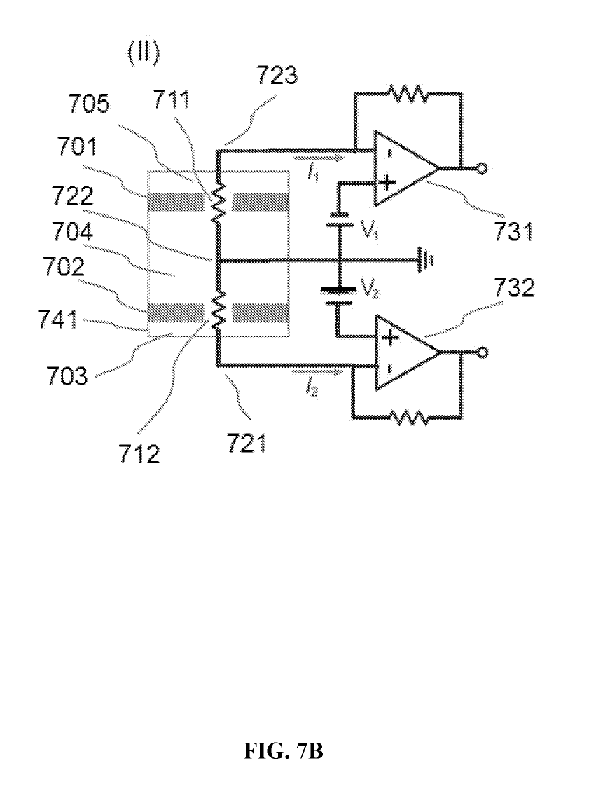

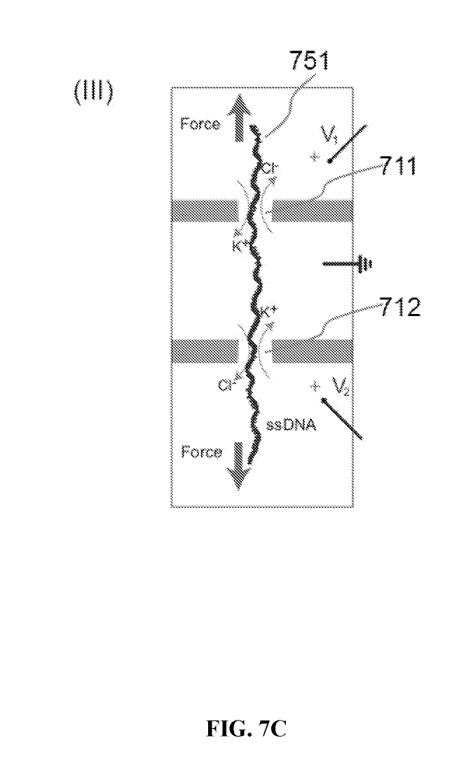

In some embodiments as shown in FIG. 7A, the device includes an upper chamber 705 (Chamber A), a middle chamber 704 (Chamber B), and a lower chamber 703 (Chamber C). The chambers are separated by two separating layers or membranes (701 and 702) each having a separate pore (711 or 712). Further, each chamber contains an electrode (721, 722 or 723) for connecting to a power supply. The annotation of upper, middle and lower chamber is in relative terms and does not indicate that, for instance, the upper chamber is placed above the middle or lower chamber relative to the ground, or vice versa.

Each of the pores 711 and 712 independently has a size that allows a small or large molecule or microorganism to pass. In one aspect, each pore is at least about 1 nm in diameter. Alternatively, each pore is at least about 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 25 nm, 30 nm, 35 nm, 40 nm, 45 nm, 50 nm, 60 nm, 70 nm, 80 nm, 90 nm, or 100 nm in diameter.

In one aspect, the pore is no more than about 100 nm in diameter. Alternatively, the pore is no more than about 95 nm, 90 nm, 85 nm, 80 nm, 75 nm, 70 nm, 65 nm, 60 nm, 55 nm, 50 nm, 45 nm, 40 nm, 35 nm, 30 nm, 25 nm, 20 nm, 15 nm, or 10 nm in diameter.

In one aspect, the pore has a diameter that is between about 1 nm and about 100 nm, or alternatively between about 2 nm and about 80 nm, or between about 3 nm and about 70 nm, or between about 4 nm and about 60 nm, or between about 5 nm and about 50 nm, or between about 10 nm and about 40 nm, or between about 15 nm and about 30 nm.

In other aspects, each pore is at least about 100 nm, 200 nm, 500 nm, 1000 nm, 2000 nm, 3000 nm, 5000 nm, 10000 nm, 20000 nm, or 30000 nm in diameter. In one aspect, each pore is 50,000 nm to 100,000 nm in diameter. In one aspect, the pore is no more than about 100000 nm in diameter. Alternatively, the pore is no more than about 50000 nm, 40000 nm, 30000 nm, 20000 nm, 10000 nm, 9000 nm, 8000 nm, 7000 nm, 6000 nm, 5000 nm, 4000 nm, 3000 nm, 2000 nm, or 1000 nm in diameter.

In some aspects, the pore has a substantially round shape. “Substantially round”, as used here, refers to a shape that is at least about 80 or 90% in the form of a cylinder. In some embodiments, the pore is square, rectangular, triangular, oval, or hexangular in shape.

Each of the pores 711 and 712 independently has a depth (i.e., a length of the pore extending between two adjacent volumes). In one aspect, each pore has a depth that is least about 0.3 nm. Alternatively, each pore has a depth that is at least about 0.6 nm, 1 nm, 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 25 nm, 30 nm, 35 nm, 40 nm, 45 nm, 50 nm, 60 nm, 70 nm, 80 nm, or 90 nm.

In one aspect, each pore has a depth that is no more than about 100 nm. Alternatively, the depth is no more than about 95 nm, 90 nm, 85 nm, 80 nm, 75 nm, 70 nm, 65 nm, 60 nm, 55 nm, 50 nm, 45 nm, 40 nm, 35 nm, 30 nm, 25 nm, 20 nm, 15 nm, or 10 nm.

In one aspect, the pore has a depth that is between about 1 nm and about 100 nm, or alternatively, between about 2 nm and about 80 nm, or between about 3 nm and about 70 nm, or between about 4 nm and about 60 nm, or between about 5 nm and about 50 nm, or between about 10 nm and about 40 nm, or between about 15 nm and about 30 nm.

In some aspects, the nanopore extends through a membrane. For example, the pore may be a protein channel inserted in a lipid bilayer membrane or it may be engineered by drilling, etching, or otherwise forming the pore through a solid-state substrate such as silicon dioxide, silicon nitride, grapheme, or layers formed of combinations of these or other materials. In some aspects, the length or depth of the nanopore is sufficiently large so as to form a channel connecting two otherwise separate volumes. In some such aspects, the depth of each pore is greater than 100 nm, 200 nm, 300 nm, 400 nm, 500 nm, 600 nm, 700 nm, 800 nm, or 900 nm. In some aspects, the depth of each pore is no more than 2000 nm or 1000 nm.

In one aspect, the pores are spaced apart at a distance that is between about 10 nm and about 1000 nm. In some aspects, the distance between the pores is greater than 1000 nm, 2000 nm, 3000 nm, 4000 nm, 5000 nm, 6000 nm, 7000 nm, 8000 nm, or 9000 nm. In some aspects, the pores are spaced no more than 30000 nm, 20000 nm, or 10000 nm apart. In one aspect, the distance is at least about 10 nm, or alternatively, at least about 20 nm, 30 nm, 40 nm, 50 nm, 60 nm, 70 nm, 80 nm, 90 nm, 100 nm, 150 nm, 200 nm, 250 nm, or 300 nm. In another aspect, the distance is no more than about 1000 nm, 900 nm, 800 nm, 700 nm, 600 nm, 500 nm, 400 nm, 300 nm, 250 nm, 200 nm, 150 nm, or 100 nm.

In yet another aspect, the distance between the pores is between about 20 nm and about 800 nm, between about 30 nm and about 700 nm, between about 40 nm and about 500 nm, or between about 50 nm and about 300 nm.

The two pores can be arranged in any position so long as they allow fluid communication between the chambers and have the prescribed size and distance between them. In one aspect, the pores are placed so that there is no direct blockage between them. Still, in one aspect, the pores are substantially coaxial, as illustrated in FIG. 7A.

In one aspect, as shown in FIG. 7A, the device, through the electrodes 721, 722, and 723 in the chambers 703, 704, and 705, respectively, is connected to one or more power supplies. In some aspects, the power supply includes a voltage-clamp or a patch-clamp, which can supply a voltage across each pore and measure the current through each pore independently. In this respect, the power supply and the electrode configuration can set the middle chamber to a common ground for both power supplies. In one aspect, the power supply or supplies are configured to apply a first voltage V₁ between the upper chamber 705 (Chamber A) and the middle chamber 704 (Chamber B), and a second voltage V₂ between the middle chamber 704 and the lower chamber 703 (Chamber C).

In some aspects, the first voltage V₁ and the second voltage V₂ are independently adjustable. In one aspect, the middle chamber is adjusted to be a ground relative to the two voltages. In one aspect, the middle chamber comprises a medium for providing conductance between each of the pores and the electrode in the middle chamber. In one aspect, the middle chamber includes a medium for providing a resistance between each of the pores and the electrode in the middle chamber. Keeping such a resistance sufficiently small relative to the nanopore resistances is useful for decoupling the two voltages and currents across the pores, which is helpful for the independent adjustment of the voltages.

Adjustment of the voltages can be used to control the movement of charged particles in the chambers. For instance, when both voltages are set in the same polarity, a properly charged particle can be moved from the upper chamber to the middle chamber and to the lower chamber, or the other way around, sequentially. In some aspects, when the two voltages are set to opposite polarity, a charged particle can be moved from either the upper or the lower chamber to the middle chamber and kept there.

The adjustment of the voltages in the device can be particularly useful for controlling the movement of a large molecule, such as a charged polymer scaffold, that is long enough to cross both pores at the same time. In such an aspect, the direction and the speed of the movement of the molecule can be controlled by the relative magnitude and polarity of the voltages as described below.

The device can contain materials suitable for holding liquid samples, in particular, biological samples, and/or materials suitable for nanofabrication. In one aspect, such materials include dielectric materials such as, but not limited to, silicon, silicon nitride, silicon dioxide, graphene, carbon nanotubes, TiO₂, HfO₂, Al₂O₃, or other metallic layers, or any combination of these materials. In some aspects, for example, a single sheet of graphene membrane of about 0.3 nm thick can be used as the pore-bearing membrane.

Devices that are microfluidic and that house two-pore microfluidic chip implementations can be made by a variety of means and methods. For a microfluidic chip comprised of two parallel membranes, both membranes can be simultaneously drilled by a single beam to form two concentric pores, though using different beams on each side of the membranes is also possible in concert with any suitable alignment technique. In general terms, the housing ensures sealed separation of Chambers A-C. In one aspect as shown in FIG. 7B, the housing would provide minimal access resistance between the voltage electrodes 721, 722, and 723 and the nanopores 711 and 712, to ensure that each voltage is applied principally across each pore.

In one aspect, the device includes a microfluidic chip (labeled as “Dual-core chip”) is comprised of two parallel membranes connected by spacers. Each membrane contains a pore drilled by a single beam through the center of the membrane. Further, the device preferably has a Teflon® housing for the chip. The housing ensures sealed separation of Chambers A-C and provides minimal access resistance for the electrode to ensure that each voltage is applied principally across each pore.

More specifically, the pore-bearing membranes can be made with transmission electron microscopy (TEM) grids with a 5-100 nm thick silicon, silicon nitride, or silicon dioxide windows. Spacers can be used to separate the membranes, using an insulator, such as SU-8, photoresist, PECVD oxide, ALD oxide, ALD alumina, or an evaporated metal material, such as Ag, Au, or Pt, and occupying a small volume within the otherwise aqueous portion of Chamber B between the membranes. A holder is seated in an aqueous bath that is comprised of the largest volumetric fraction of Chamber B. Chambers A and C are accessible by larger diameter channels (for low access resistance) that lead to the membrane seals.

A focused electron or ion beam can be used to drill pores through the membranes, naturally aligning them. The pores can also be sculpted (shrunk) to smaller sizes by applying a correct beam focusing to each layer. Any single nanopore drilling method can also be used to drill the pair of pores in the two membranes, with consideration to the drill depth possible for a given method and the thickness of the membranes. Predrilling a micro-pore to a prescribed depth and then a nanopore through the remainder of the membranes is also possible to further refine the membrane thickness.

In another aspect, the insertion of biological nanopores into solid-state nanopores to form a hybrid pore can be used in either or both pores in the two-pore method. The biological pore can increase the sensitivity of the ionic current measurements, and is useful when only single-stranded polynucleotides are to be captured and controlled in the two-pore device, e.g., for sequencing.

By virtue of the voltages present at the pores of the device, charged molecules can be moved through the pores between chambers. Speed and direction of the movement can be controlled by the magnitude and polarity of the voltages. Further, because each of the two voltages can be independently adjusted, the direction and speed of the movement of a charged molecule can be finely controlled in each chamber.

One example concerns a charged polymer scaffold, such as a DNA, having a length that is longer than the combined distance that includes the depth of both pores plus the distance between the two pores. For example, a 1000 by dsDNA is about 340 nm in length, and would be substantially longer than the 40 nm spanned by two 10 nm-deep pores separated by 20 nm. In a first step, the polynucleotide is loaded into either the upper or the lower chamber. By virtue of its negative charge under a physiological condition at a pH of about 7.4, the polynucleotide can be moved across a pore on which a voltage is applied. Therefore, in a second step, two voltages, in the same polarity and at the same or similar magnitudes, are applied to the pores to move the polynucleotide across both pores sequentially.

At about the time when the polynucleotide reaches the second pore, one or both of the voltages can be changed. Since the distance between the two pores is selected to be shorter than the length of the polynucleotide, when the polynucleotide reaches the second pore, it is also in the first pore. A prompt change of polarity of the voltage at the first pore, therefore, will generate a force that pulls the polynucleotide away from the second pore as illustrated in FIG. 7C.

Assuming that the two pores have identical voltage-force influence and |V₁|=|V₂|+δV, the value δV>0 (or <0) can be adjusted for tunable motion in the V₁| (or V₂) direction. In practice, although the voltage-induced force at each pore will not be identical with V₁=V₂, calibration experiments can identify the appropriate bias voltage that will result in equal pulling forces for a given two-pore chip; and variations around that bias voltage can then be used for directional control.

If, at this point, the magnitude of the voltage-induced force at the first pore is less than that of the voltage-induced force at the second pore, then the polynucleotide will continue crossing both pores towards the second pore, but at a lower speed. In this respect, it is readily appreciated that the speed and direction of the movement of the polynucleotide can be controlled by the polarities and magnitudes of both voltages. As will be further described below, such a fine control of movement has broad applications.

Accordingly, in one aspect, provided is a method for controlling the movement of a charged polymer scaffold through a nanopore device. The method entails (a) loading a sample comprising a charged polymer scaffold in one of the upper chamber, middle chamber or lower chamber of the device of any of the above embodiments, wherein the device is connected to one or more power supplies for providing a first voltage between the upper chamber and the middle chamber, and a second voltage between the middle chamber and the lower chamber; (b) setting an initial first voltage and an initial second voltage so that the polymer scaffold moves between the chambers, thereby locating the polymer scaffold across both the first and second pores; and (c) adjusting the first voltage and the second voltage so that both voltages generate force to pull the charged polymer scaffold away from the middle chamber (voltage-competition mode), wherein the two voltages are different in magnitude, under controlled conditions, so that the charged polymer scaffold moves across both pores in either direction and in a controlled manner.

To establish the voltage-competition mode in step (c), the relative force exerted by each voltage at each pore is to be determined for each two-pore device used, and this can be done with calibration experiments by observing the influence of different voltage values on the motion of the polynucleotide, which can be measured by sensing known-location and detectable features in the polynucleotide, with examples of such features detailed later in this disclosure. If the forces are equivalent at each common voltage, for example, then using the same voltage value at each pore (with common polarity in upper and lower chambers relative to grounded middle chamber) creates a zero net motion in the absence of thermal agitation (the presence and influence of Brownian motion is discussed below). If the forces are not equivalent at each common voltage, achieving equal forces involves the identification and use of a larger voltage at the pore that experiences a weaker force at the common voltage. Calibration for voltage-competition mode can be done for each two-pore device, and for specific charged polymers or molecules whose features influence the force when passing through each pore.

In one aspect, the sample containing the charged polymer scaffold is loaded into the upper chamber and the initial first voltage is set to pull the charged polymer scaffold from the upper chamber to the middle chamber and the initial second voltage is set to pull the polymer scaffold from the middle chamber to the lower chamber. Likewise, the sample can be initially loaded into the lower chamber, and the charged polymer scaffold can be pulled to the middle and the upper chambers.

In another aspect, the sample containing the charged polymer scaffold is loaded into the middle chamber; the initial first voltage is set to pull the charged polymer scaffold from the middle chamber to the upper chamber; and the initial second voltage is set to pull the charged polymer scaffold from the middle chamber to the lower chamber.

In one aspect, the adjusted first voltage and second voltage at step (c) are about 10 times to about 10,000 times as high, in magnitude, as the difference/differential between the two voltages. For instance, the two voltages can be 90 mV and 100 mV, respectively. The magnitude of the two voltages, about 100 mV, is about 10 times of the difference/differential between them, 10 mV. In some aspects, the magnitude of the voltages is at least about 15 times, 20 times, 25 times, 30 times, 35 times, 40 times, 50 times, 100 times, 150 times, 200 times, 250 times, 300 times, 400 times, 500 times, 1000 times, 2000 times, 3000 times, 4000 times, 5000 times, 6000 times, 7000 times, 8000 times or 9000 times as high as the difference/differential between them. In some aspects, the magnitude of the voltages is no more than about 10000 times, 9000 times, 8000 times, 7000 times, 6000 times, 5000 times, 4000 times, 3000 times, 2000 times, 1000 times, 500 times, 400 times, 300 times, 200 times, or 100 times as high as the difference/differential between them.

In one aspect, real-time or on-line adjustments to the first voltage and the second voltage at step (c) are performed by active control or feedback control using dedicated hardware and software, at clock rates up to hundreds of megahertz. Automated control of the first or second or both voltages is based on feedback of the first or second or both ionic current measurements.

### Sensors

As discussed above, in various aspects, the nanopore device further includes one or more sensors to carry out the identification of the binding status of the binding motifs.

The sensors used in the device can be any sensor suitable for identifying a target analyte, such as a polymer. For instance, a sensor can be configured to identify the polymer (e.g., a polymer scaffold) by measuring a current, a voltage, a pH value, an optical feature, or residence time associated with the polymer. In other aspects, the sensor may be configured to identify one or more individual components of the polymer or one or more components bound to the polymer. The sensor may be formed of any component configured to detect a change in a measurable parameter where the change is indicative of the polymer, a component of the polymer, or preferably, a component bound to the polymer. In one aspect, the sensor includes a pair of electrodes placed at two sides of a pore to measure an ionic current across the pore when a molecule or other entity, in particular a polymer scaffold, moves through the pore. In certain aspects, the ionic current across the pore changes measurably when a polymer scaffold segment passing through the pore is bound to a probe, such as a label, a fusion molecule and/or fusion molecule-target analyte complex. Such changes in current may vary in predictable, measurable ways corresponding with, for example, the presence, absence, and/or size of the fusion molecules and target analytes present.

In a preferred embodiment, the sensor comprises electrodes which apply voltage and are used to measure current across the nanopore. Translocations of molecules through the nanopore provides electrical impedance (Z) which affects current through the nanopore according to Ohm's Law, V=IZ, where V is voltage applied, I is current through the nanopore, and Z is impedance. The result when a molecule translocates through a nanopore in an electrical field (e.g., under an applied voltage) is an electrical signal that may be correlated to the molecule passing through the nanopore upon further analysis of the current signal.

When residence time measurements from the electrical signal are used, the size of the component can be correlated to the specific component based on the length of time it takes to pass through the sensing device.

In an embodiment, a sensor is provided in the nanopore device that measures an optical feature of the polymer, a component (or unit) of the polymer, or a component bound to the polymer. One example of such measurement includes the identification of an absorption band unique to a particular unit by infrared (or ultraviolet) spectroscopy.

In some embodiments, the sensor is an electric sensor. In some embodiments, the sensor detects a fluorescent detection means when the target analyte or the detectable label passing through has a unique fluorescent signature. A radiation source at the outlet of the pore can be used to detect that signature.

Analysis of Data from Nanopore Detection

Described herein are methods of encoding one or more bit(s) of information by placing one or more molecules along a polymer scaffold so that information encoded in the polymer scaffold can be retrieved by passing the polymer scaffold through a nanopore and examining the current impedance signatures curves.

A molecule that is used on a polymer for the sole purpose of storing information is called a “label.” A label is considered “unique” if it causes a signature curve that can be differentiated against other labels (synthetic) or molecules (natural) on that same polymer. A single polymer scaffold can contain one or more labels to represent increasingly more complex information. Therefore, a synthetic polymers bound to one or more labels reside in a reservoir without the presence of natural molecules, this method can be used to store arbitrary amounts of static information for later recall.

A method of data retrieval of data encoded in a polymer scaffold is performed in a device that contains one or more nanopores, and a chamber with synthetic polymers that contain labels. A voltage is applied, causing negatively charged molecules, including the polymer scaffold, to pass through the nanopore. As molecules pass through the pore, events are generated, and the data is analyzed by the software to discern the presence of known signature curves. If a (portion of the) signature curve matches one of the known labels, the rest of the event is analyzed for more signature curves, and they are assembled in the same order in which they assembled on the polymer. The software determines if/how to translate the information captured into whatever intended purpose the software serves. (e.g., different signature curves may map to different letters of an alphabet, or pixel values, or MIDI data.)

When synthetic polymers are used in reservoirs that also contain molecules found in nature, the synthetic polymer must be designed in such a manner that the event is assured to be different from that which would be generated by any of the natural molecules in the same reservoir. A synthetic polymer may also have additional sites that have binding molecules intended to capture natural analytes that may reside in the reservoir.

In an embodiment, the method of identifying an analyte from a bulk solution is performed on a device that contains one or more nanopores, and a chamber with synthetic polymers that contain labels and fusion molecules intended to capture one or more analytes. A microfluidic channel may be included in the device that allows sample fluid from a natural source to enter into the reservoir chamber. As the molecules from the sample interact with the synthetic polymers, target analytes will bind with the fusion molecules. A voltage is then applied to the sample mixture, causing negatively charged molecules, such as polymer scaffolds, to pass through the nanopore. As molecules pass through the pore, events are generated, and the data is analyzed by the software to discern the presence of known signature curves.

If the software does not identify any of the signature curves from the set of known labels, the entire event is discarded. If a signature curve matches one of the known labels, the rest of the event is analyzed for more signature curves. If the software determines that the polymer has a binding molecule, that molecule's signature curve is analyzed to see if a target analyte was bound to the binder.

In another embodiment optical signals may be used instead of current impedance measurements to discern the presence of molecules along the polymer scaffold. In an embodiment, the method of detecting optical signals from a polymer scaffold to read data encoded on the polymer scaffold is performed in a nanopore device. Voltage is applied to drive negatively charged polymers through the nanopore. An optical sensor is used in the device to capture an optical measurement within a fixed field of view that may reside at or adjacent to the nanopore. The optical measurement comprises a measure of light detected within a fixed period of time. This measurement may include, but not be limited to, one or more of individual values, such as color, luminescence, and intensity. The method can be used to detect a tagged molecule in a chemical complex that has been modified in such a manner to generate an optical signal that an optical sensor will detect, providing a particular optical measurement.

An “optical event” is a set of optical measurements captured by the sensor from a single polymer scaffold that may contain one or more tagged molecules. Because the sensor cannot discern between the beginning and end of a polymer using optics, the ends of the polymer may be detected by current impedance measurements to determine when a polymer enters (e.g., when the measurement's y value deviates beyond an open channel threshold, or adding tagged molecules that will produce a known optical measurement when bound at each end of the polymer. An optical signature is a collection of optical measurements within an optical event where the software analyzes them in such a manner that it determines it has read a unique abstract value. Since a polymer may have one or more molecules bound to it, an event may contain one or more signatures. A symbol is the assembly of one or more optical signatures within an event so as to comprise a single abstraction. E.g., “red, green, red, green” may equate to the letter “A”

In several of the embodiments, the electrical signal provided may be compared against a database that correlates a molecule or complex with an electrical signal. This molecule or complex may be any of the entities discussed herein as capable of detecting via current impedance upon translocation through the nanopore, or other methods of detection, such as optical measurements. A database may be generated by reading the electrical signals provided by a homogenous population. Analysis of a homogenous population of polymer scaffolds bound to probes, which may further be bound to analytes or other entities is useful for assessing the variation in signal pattern generated and determining a reference signal for that coded molecule. Events and electrical signals from a sample combined with the same polymer scaffold and probes can then be analyzed and compared to the database comprising the reference signals correlated to an analyte or polymer scaffold identification and/or quantitation.

### EXAMPLES

The present technology is further defined by reference to the following example and experiments. It will be apparent to those skilled in the art that many modifications may be practiced without departing from the scope of the current invention

### Example 1—DNA Alone in Solid-State Nanopore Experiment

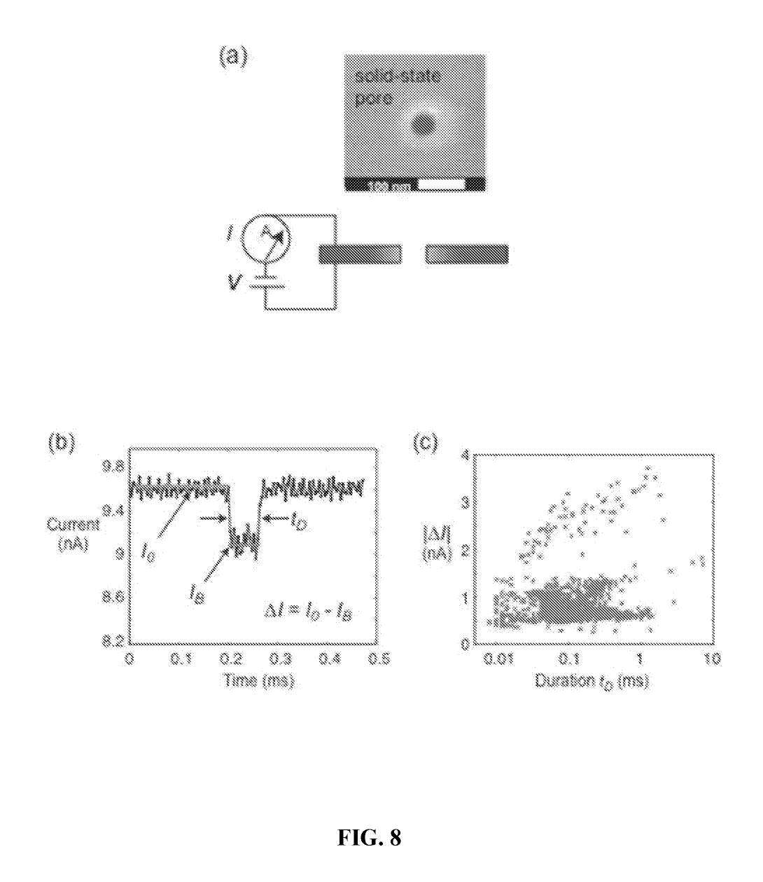

Nanopore instruments use a sensitive voltage-clamp amplifier to apply a voltage V across the pore while measuring the ionic current l₀ through the open pore (FIG. 8, panel (a)). When a single charged molecule such as a double-stranded DNA (dsDNA) is captured and driven through the pore by electrophoresis (FIG. 8, panel (b)), the measured current shifts from l₀ to l_{B}, and the shift amount Δl=l₀−l_{B} and duration t_{D} are used to characterize the event. After recording many events during an experiment, distributions of the events (FIG. 8, panel (c)) are analyzed to characterize the corresponding molecule. In this way, nanopores provide a simple, label-free, purely electrical single-molecule method for biomolecular sensing.

In the DNA experiment shown in FIG. 8, the single nanopore fabricated in silicon nitride (SiN) substrate is a 40 nm diameter pore in 100 nm thick SiN membrane (FIG. 8, panel (a)). In FIG. 8(b), the representative current trace shows a blockade event caused by a 5.6 kb dsDNA passing in a single file manner (unfolded) through an 11 nm diameter nanopore in 10 nm thick SiN at 200 mV and 1M KCl. The mean open channel current is l₀=9.6 nA, with mean event amplitude l_{B}=9.1 nA, and duration t_{D}=0.064 ms. The amplitude shift is Δ/=l₀−l_{B}=0.5 nA. In FIG. 6C, the scatter plot shows |Δ/| vs. t_{D} for all 1301 events recorded over 16 minutes.

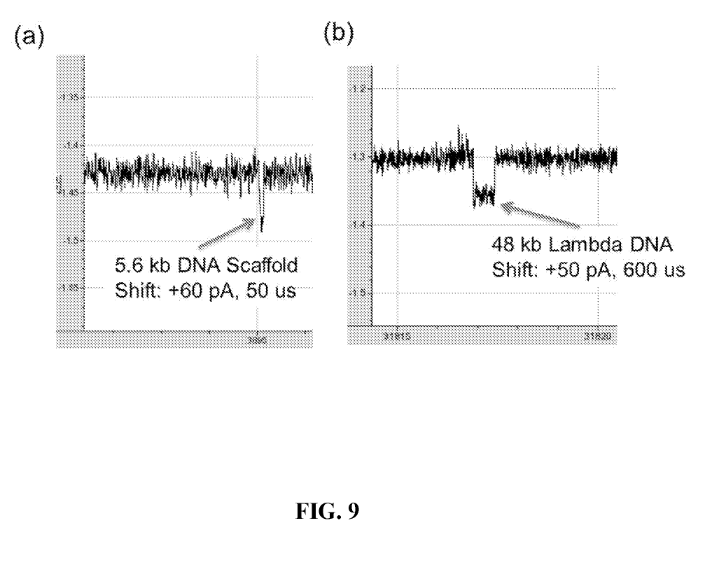

In the DNA experiment shown in FIG. 9, dsDNA alone causes current enhancement events at 100 mM KCl. This was shown in the published research of Smeets, Ralph M M, et al. “Salt dependence of ion transport and DNA translocation through solid-state nanopores.” Nano Letters 6.1 (2006): 89-95). The study showed that, while the amplitude shift Δl=l₀−l_{B}>0 for KCl concentration above 0.4 M, the shift has opposite polarity (Δl<0) for KCl concentration below 0.4 M. As this is a negative voltage experiment (−200 mV) with KCl concentration below 0.4 M, we see that the DNA event has the same polarity (416) relative to the baseline (415) as shown in FIG. 4C.

### Example 2—Binding of PNA to dsDNA Scaffold and Detection in a Nanopore

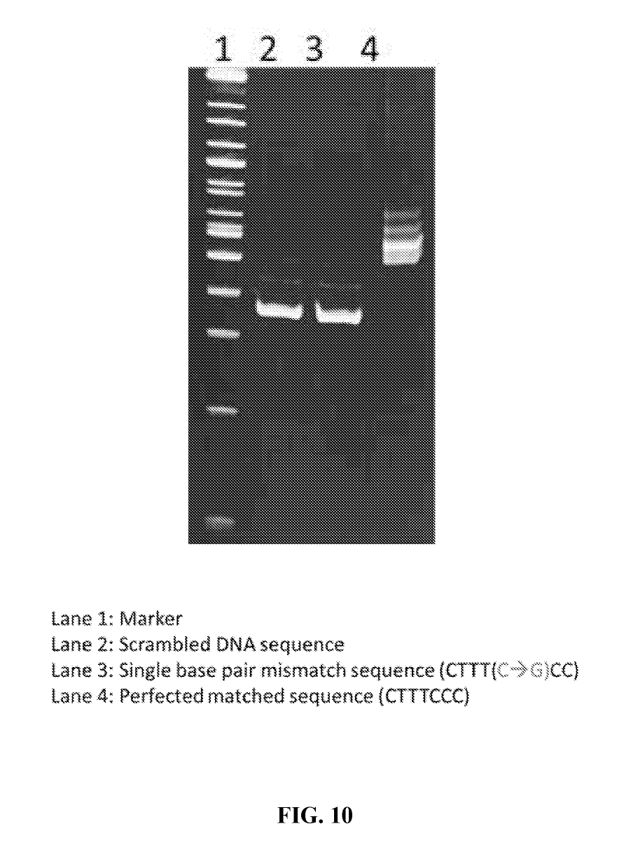

To show that the bisPNA molecule is specific for its target sequence, binding experiments were performed using a scrambled 324 bp dsDNA fragment, a 324 bp dsDNA fragment with a complementary sequence to the bisPNA except for a single base pair mismatch sequence, and a 324 bp dsDNA fragment with a perfectly matched complementary sequence. FIG. 10 shows that only the perfect match sequence shows bisPNA binding. Thus, the bisPNA scaffold binding domain binds to the label binding domain on the bisPNA with high stringency and selectivity.

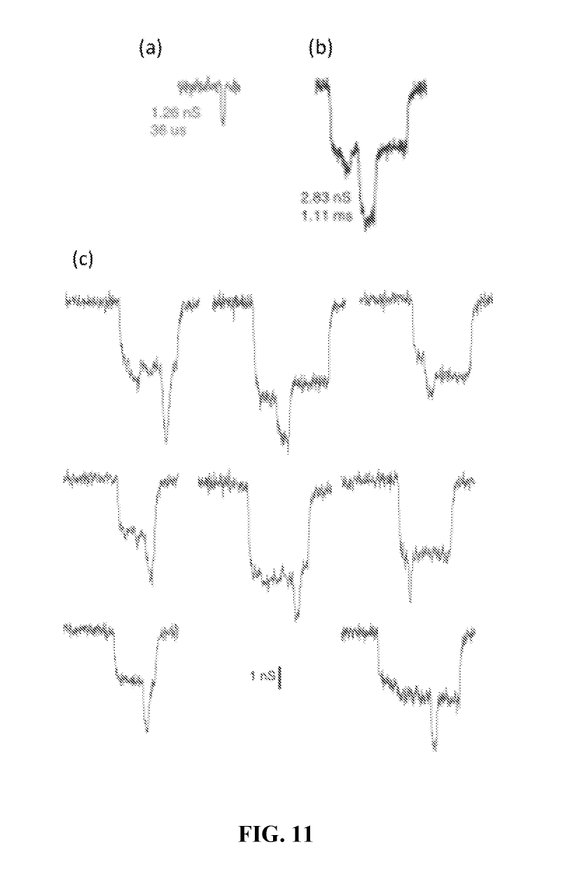

In addition, a 4-6 nm nanopore in a nanopore device is capable of detecting the bisPNA label on the dsDNA scaffold. As shown in FIG. 11, a nanopore assay as described herein is capable of detecting the (a) absence, or (b,c) presence of a bis-PNA label to the target sequence of a 324 bp dsDNA. With the 7 bp target sequence located in the middle, representative events show a distinct pattern not observed otherwise.

### Example 3—Detection of PNA Bound to a Detectable Tag in a Nanopore

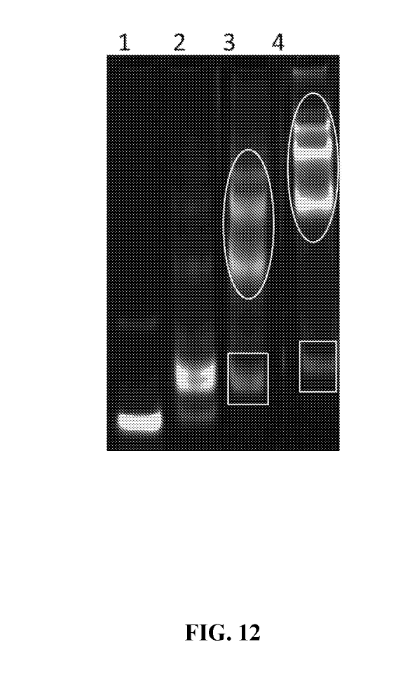

The formation of label-DNA complexes, where the label comprises a detectable tag was shown as follows. dsDNA was incubated with bis-PNA molecules comprising either 5 kDa and 10 kDa PEG as a detectable tag. Formation of the label-dsDNA complex was observed in a gel as shown in FIG. 12. In lane 1, DNA alone is run as a control. A 324 bp DNA fragment was bound by bisPNA that contained no PEG (lane 2), bound by a bisPNA that contained 5 kDa PEG (lane 3), or bound by a bisPNA that had a 10 kDa PEG conjugated. The triple banding pattern in lanes 3 and 4 (circled) are due to the different conformations the PNA takes when binding. The lowest band in lanes 3 and 4 (square) is likely DNA bound by PNA that was not PEG labeled.

Next, we determined whether we could detect the PNA labels with PEG detectable tags bound to dsDNA polymer scaffold in a nanopore assay. We ran a sample comprising 324 bp dsDNA with a bis-PNA binding domain and either (a) ZERO-payload, (b) 5 kDa PEG-payload, or (c) 10 kDa PEG-payload bits bound to bis-PNA.

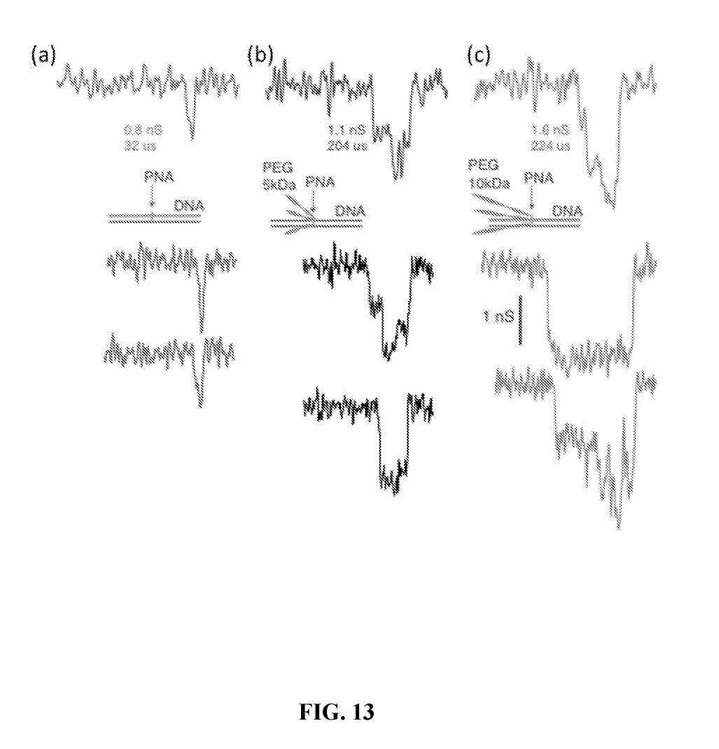

As shown in FIG. 13 a nanopore was able to discriminate DNA alone and DNA bound by bisPNA with ZERO, PEG 5 k and PEG 10 k payloads bound to 324 bp dsDNA. With the 7 bp target sequence located in the middle, representative events show a distinct pattern observed distinct for each bit, using 15-35 nm diameter nanopores. Each PNA has 3 PEGs of the stated size as the detectable tag. All events are on a common vertical scale for current amplitude. These events were collected from the same experiment, showing simultaneous bit discrimination

### Example 4—Probe Multiplexing

We determined that an individual polymer scaffold is capable of reliably binding multiple labels. Such labels can generate distinct electrical signatures when bound to a scaffold and passed through an appropriately designed nanopore. We ran DNA, DNA bound to a single PNA bound to PEG 5 k, and DNA bound to a single PNA bound to PEG 5 k.

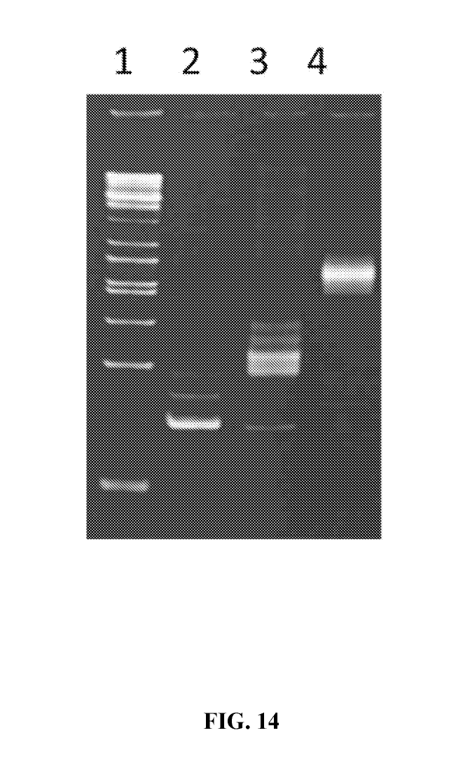

Gel shift shows gammaPNA-PEG 5 kDa can bind to the same sequence. As shown in FIG. 14, a gel shift assay shows that a single or two gammaPNA-PEG 5 kDa can bind to the same fragment molecule. Lane 1: Marker. Lane 2: DNA fragment only. Lane 3: DNA fragment+1×PNA-5 k. Lane 4: DNA fragment+2×PNA-5 k. Thus, multiple probes, such as labels or fusion molecules can bind to the same scaffold to allow multiplexing.

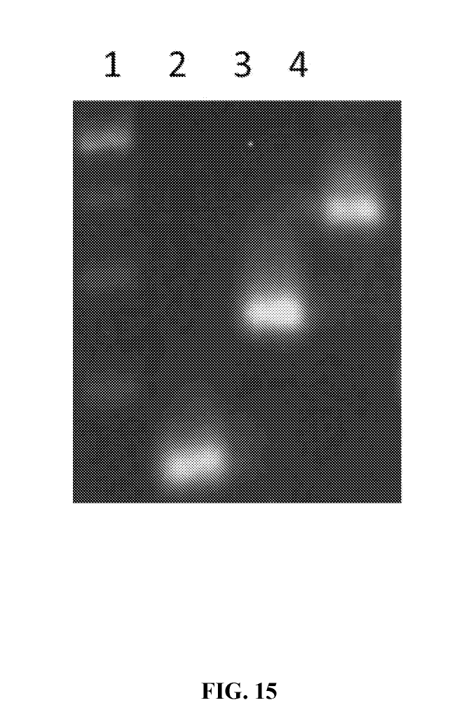

We also showed that a polymer scaffold is capable of binding a plurality of monostreptavidin proteins as probes. We generated DNA fragments comprising biotinylated ends using biotinylated PCR, then incubated the DNA with monostreptavidin. The gel shift shown in FIG. 15 shows a DNA fragment can be reliably tagged with a plurality of monostreptavidin proteins. Lane 1 shows a marker. Lane 2 shows DNA fragment only. Lane 3 shows DNA fragment+1× monostreptavidin. Lane 4 shows DNA fragment+2× monostreptavidin. Thus, DNA fragments generated by biotinylated PCR primers can be bound by a plurality of streptavidin protein labels.

Detection of Multiple Sites of a Sequence in 5.6 kb dsDNA

A linear 5.6 kbp dsDNA molecule was engineered to contain a unique 12 bp sequence (uSeq1) interspersed at 25 sites within the DNA. The purpose of this repetition is to boost the sensing signal for each scaffold, since the more occupied PNA sites there are, the longer the nanopore current is impeded, yielding a more easily detected signature.

Instead of using bis-PNA as the sequence-specific binding molecule, we used the smaller and more versatile γPNA. Positive detection and localization of these smaller PNAs is possible, but required a precision sub-4 nm pore, and was shown to work with a salt gradient. In the absence of a salt gradient and with a larger pore (11 nm diameter, 10 nm membrane), we sought to demonstrate positive detection of the presence of the label by adding a detectable tag to each PNA. To provide the option of increasing label size, the PNA had three biotin molecules incorporated via coupling to free amines on the backbone Lysine amino acid.

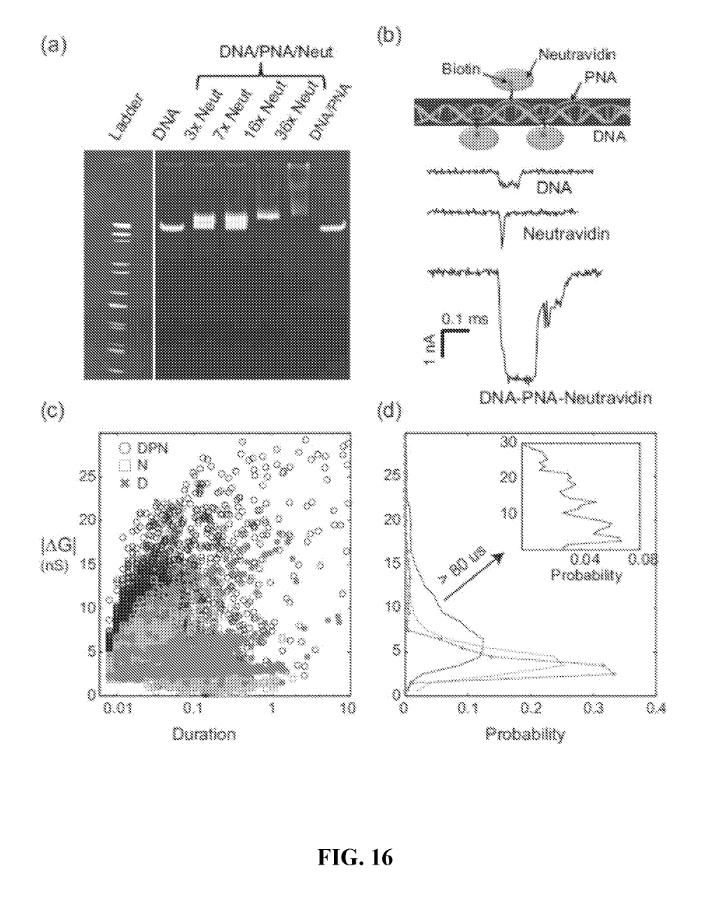

This DNA-PNA-Neutravidin (DPN) reagent was tested in using a nanopore 11 nm in diameter formed by dielectric breakdown in a 10 nm membrane. FIG. 16, panels (b-d) show data comparing ΔG versus duration distributions for events from three separate experiments conducted sequentially on the same pore: DNA alone, Neutravidin alone, and DPN reagents.

The largest ΔG events in the DPN experiment are attributed to DPN complexes (FIG. 16, panel (b)), providing a simple criteria for tagging events as having the target 12 bp sequence. The mathematical criteria derived above can be used to assess confidence in detection. Using the criteria ΔG>20 nS, 390 of the events in the DPN experiment are tagged resulting in

Q(p)=9.29%. In the prior control experiments, only 0.46% of D and 0.16% of N events are detected. Applying the mathematical criteria above, with Q=Fraction of N-flagged events, the 99% confidence interval is Q=9.29+/−1.15% for this data set. Since 9.29%>0.46% (the max false-positive %) well within the 99% confidence interval for Q, we have a positive test result, and in under 8 minutes of data gathering. In fact the same 99% confidence is achieved for this data set with only the first 60 seconds of the data. The gel shift (FIG. 16, panel (a)) shows that scaffold DNA migration is retarded in a Neutravidin dependent manner and guided us to using the 10× concentration in this preliminary experiment, as it appeared all DNA is bound and a nearly homogenous population is created.

### Example 5—Detection of an Analyte in Human Blood Using a dsDNA Scaffold

We ran 1:20 diluted samples of human blood (whole and serum) with a control polymer scaffold in the nanopore device. conducted nanopore experiments that incorporates human blood (whole and serum) with a control molecule. Several generic events from the current impedance data from background molecules in the mixed sample were generated. Rec-A coated DNA was then added to the sample to test the ability to distinguish RecA coated DNA from background molecules based on the electrical signal generated upon translocation through the nanopore. The ability to detect RecA coated DNA in the sample using the nanopore was hindered due to overlap between electrical signals from RecA coated DNA and background molecules in the blood sample.

However, a labeled polymeric scaffold, such as PNA with a detectable tag bound to dsDNA, provides an electrical signal that is unique from the background molecules in blood when these molecules translocate through the nanopore under an applied voltage. Therefore, we will generate a polymer scaffold that comprises label binding domains and fusion molecule binding domains. The label binding domains attach to labels optionally comprising a detectable tag that will provide a unique electrical signal to distinguish the polymer scaffold from background molecules. Then, the detection of a target analyte by an electrical signal present on the current event generated by the polymer scaffold can be performed by analysis of the electrical signal provided by the bound or unbound fusion molecule.

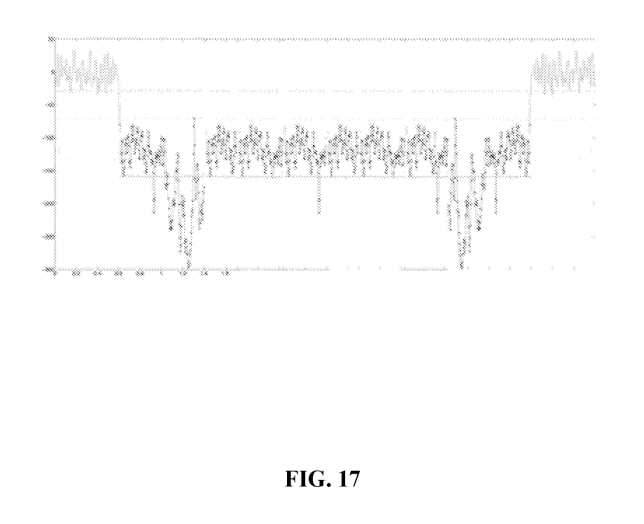

Therefore, the use of labels and binding label domains on the polymer scaffold will be used to identify scaffolds that have one or more target analytes. FIG. 17 shows a prototype illustration of an electrical signal generated upon the translocation of a polymer scaffold with PNA molecules bound to 5K PEGs on either end of the polymer scaffold, with a fusion molecules and target analyte in the middle, through the nanopore. The unique signature provided by this construct is not present in bulk samples. The molecule is too long and unique features on either end are too uniform, so that there is very low probability that an overlapping electrical signal would be produced by a natural molecule that is not the engineered polymer scaffold. Therefore, the fusion molecule in the center of the electrical signal in FIG. 17 may be specifically analyzed for the absence or presence of an analyte.

To optimize the performance of the system so that it can be computationally simple, we can use known algorithms to smooth out specific events or electrical signals, such as those correlated with a polymer scaffold translocation. For example, we can use moving averages, Bollinger Bands, among others, to infer the shape of data. We will use these algorithms to compare shapes of all events from the nanopore and quickly isolate those that resolve within a threshold. We can also use a unique compression algorithm tuned for certain electrical signals to reduce storage size of the data.

Additional polymer scaffolds for analyte detection comprising a defined sequence of label binding domains and at least one fusion molecule binding domain for analyte detection will be generated using this method, which will allow us to discriminate from all background events. Additionally, little or no sample prep is needed for assays where the target analytes in solution in the sample. However, some sample prep to extract that targets embedded in, e.g., cells or soil may be performed to liquefy the sample or isolate certain portions of the sample.

### Example 6—Detection of Label and Presence or Absence of Target Using a dsDNA Scaffold

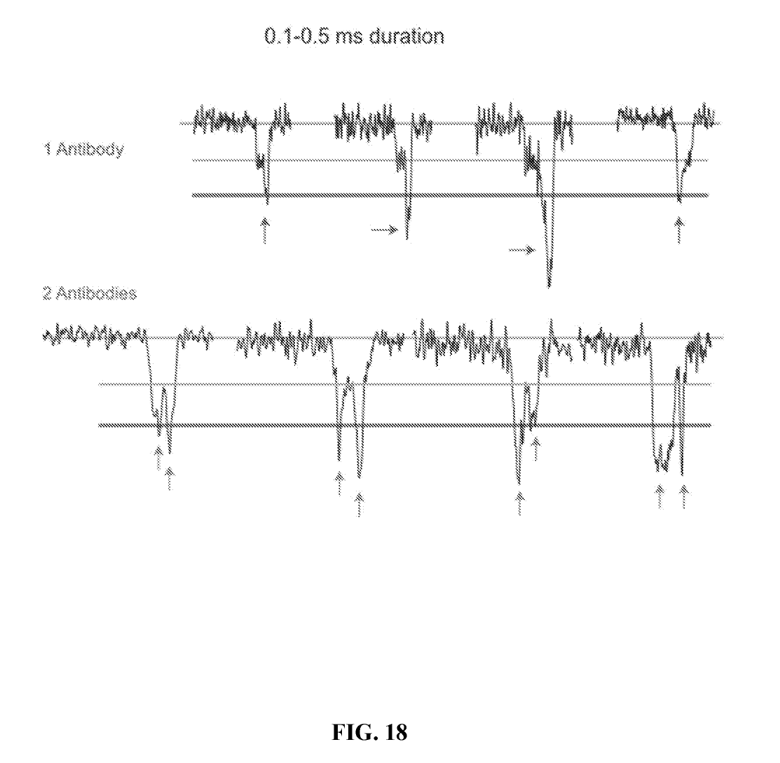

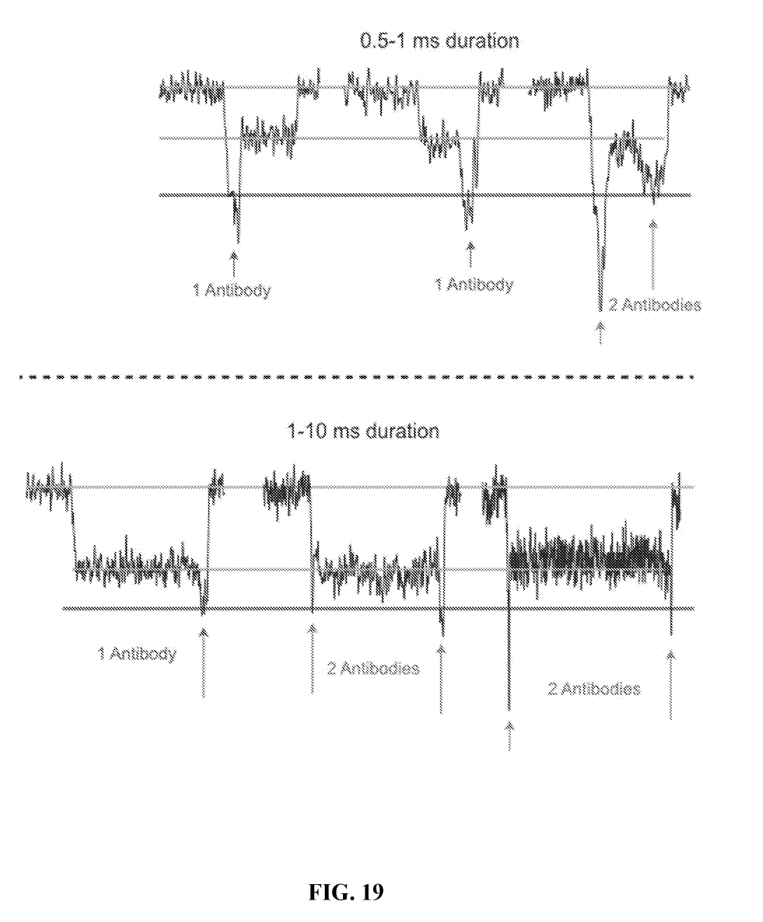

The polymer scaffold comprises probe binding domains that may reside on the ends of the DNA as chemical modification to which labels or analyte detection molecules are chemically tethered or bound. FIG. 18 shows a dsDNA scaffold with events 0.1-0.5 ms, and with a single antibody acting as a label at one end, and the absence or presence of a separate target analyte antibody at the other end. Event signatures have a single “spike” when only the label antibody is present, and two “spikes” when the target analyte antibody is present, signaling detection of the target for that molecule. These spikes are identified by automated algorithms, that quantitate the spikes as have distinct amplitude levels and durations. The algorithm can work on shorter or longer duration events, and with steps of shorter or longer duration within events. FIG. 19 shows a dsDNA scaffold with longer lasting events (0.5-10 ms), still with a single antibody acting as a label at one end, and the absence or presence of a separate target analyte antibody at the other end. As before, event signatures have a single “spike” when only the label antibody is present, and two “spikes” when the target analyte antibody is present, signaling detection of the target for that molecule. The detections and event classifications are all done by algorithms in software, and can be in real-time or offline after experimentation.

### Example 7—Creation of Reagents with PNA-PEG Payloads and PNA-Peptide Fusion Molecules for Target HIV Antibody Binding

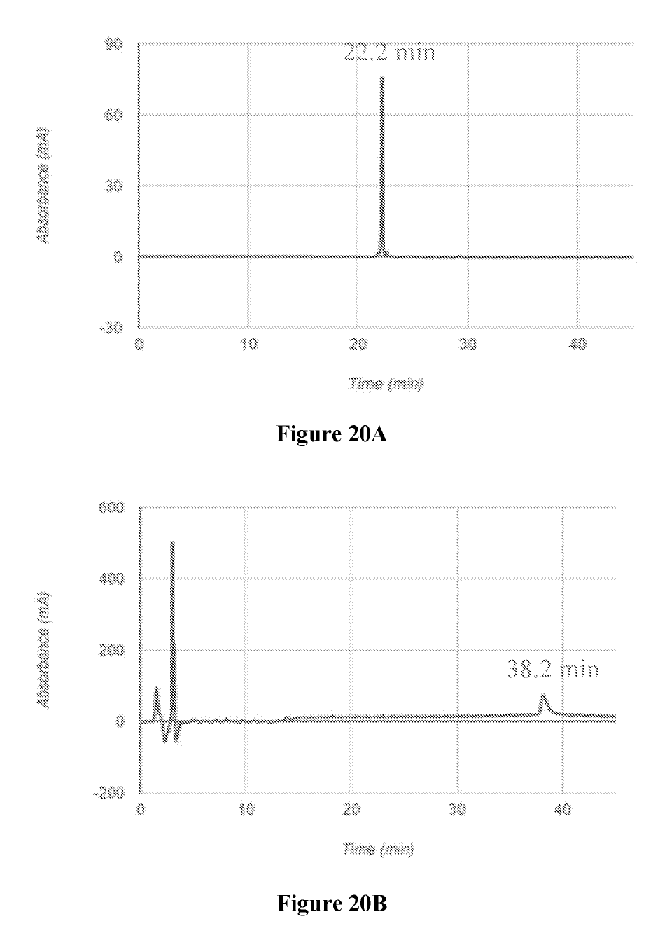

FIG. 20 depicts reverse phase high-performance liquid chromatography (RP-HPLC) chromatograms of reagents used in the PNA-PEG conjugations. Specifically, FIG. 20A shows the absorbance trace of the PNA alone at a wavelength of 270 nm, indicating that the molecule elutes at 22.2 minutes. FIG. 20B shows the absorbance trace and elution profile of the 10 kDa PEG molecule at 214 nm, indicating that the molecule elutes from the reverse phase column at 38.2 minutes. PEG does not absorb at 270 nm, and therefore 214 nm was used to detect its presence.

### Conjugation and Purification of PNA-PEG

HPLC purified peptide nucleic acid (PNA) containing a cysteine (Cys) residue for downstream conjugation was purchased directly from a commercial vendor (Panagene, South Korea) of the following sequence:
KK-O-TCC CCT CCT TTT-O-O-O-TTT TJJ TJJ JJT-O-KK-OO-Cys

where K is the amino acid lysine, O is a polyethylene glycol (PEG) linker, and J is pseudo-isocytosine.

First, 40 uL of 20 uM PNA was allowed to incubate with 200 uM of a polydisperse 10 kDa maleimide-PEG solution (Creative PEGWorks, Cat #PSB-233) for a period of 20 minutes at room temperature (10 mM sodium phosphate, pH 7.4). Following the brief incubation, the sample was submitted to reverse phase chromatography in order to isolate the reaction product (Vydac column, Cat #218TP54). Because reverse phase separates molecules according to their hydrophobicity, samples were injected under a high percentage of Buffer A (water with 0.1% trifluoroacetic acid) and allowed to elute with increasing concentrations of the hydrophobic Buffer B (acetonitrile with 0.1% trifluoroacetic acid) according to the following program:

| Time (min) | % Buffer B |
|---|---|
| 0 | 5 |
| 10 | 5 |
| 46.6 | 60 |
| 51.6 | 90 |
| 61.6 | 90 |
| 71.6 | 5 |
| 86.6 | 5 |

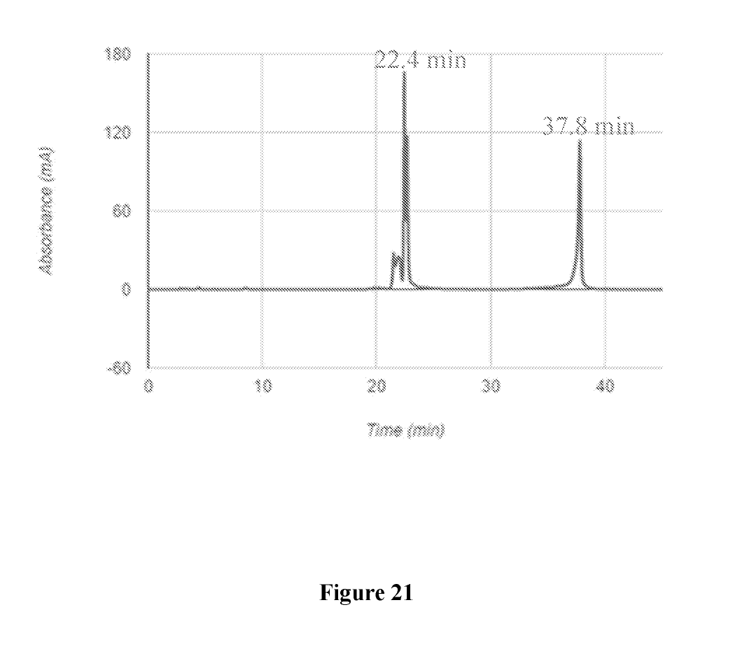

The peak representative of the conjugate product eluting at 37.8 min (FIG. 21), was collected and dried down for downstream characterization via mass spectrometry, as well as binding to DNA and subsequent nanopore analysis.

### Mass Spec Analysis of Purified PNA-PEG Conjugate

Specifically, MALDI-TOF-MS analysis was conducted on a Bruker AutoFlex III instrument, utilizing the dried droplet technique. First, 20 μL of ultrapure water was added to dissolve the contents of the previously HPLC purified and dried down sample. The resulting solution contained a concentration of approximately 20 uM purified conjugate. MALDI-MS samples were prepared by mixing a 2 L droplet of a saturated solution of sinapinic acid matrix with a 2 μL droplet of previously reconstituted sample in a plastic Eppendorf tube using a solvent system composed of acetonitrile:water at a 1:1 ratio with 0.5% trifluoroacetic acid.

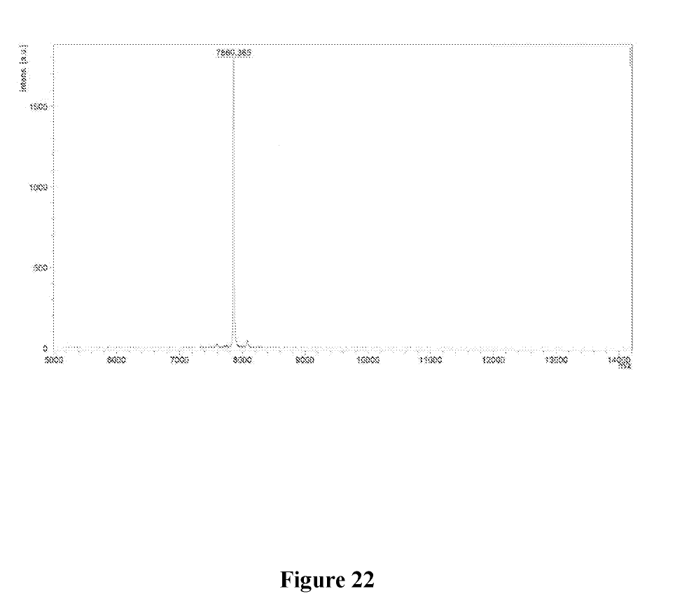

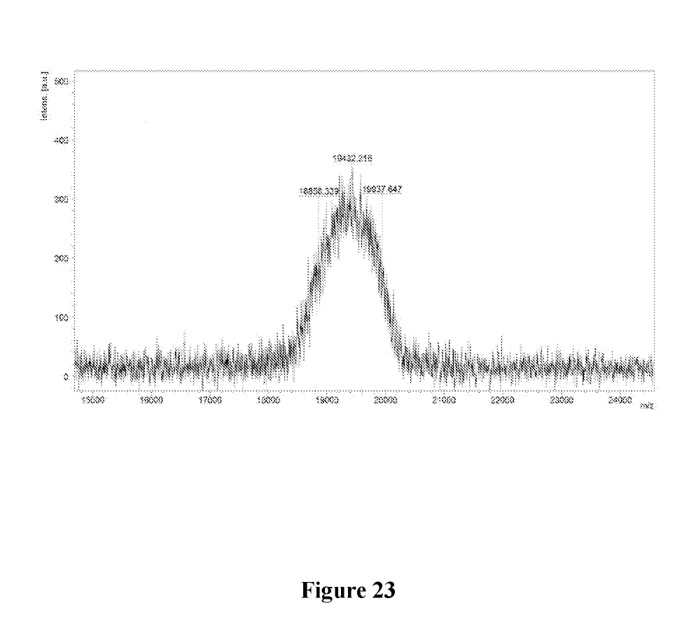

The samples were subsequently vortexed for 1 minute, and a 1 μL droplet of the mixed sample solution was deposited on the stainless steel MALDI plate, allowed to air dry, and then the MALDI plate was inserted into the MALDI-MS for analysis. The MALDI-TOF instrument was calibrated using the Bruker Daltonics Protein Mix Standard 1 utilizing four data points in the mass range 14 kDa to 40 kDa. Mass spectra was acquired with a minimum of 5000 shots per spectrum. FIG. 22 shows the MALDI-TOF mass spectra of the PNA molecule alone, indicating a molar mass of 7860.365 daltons. FIG. 23 depicts the MALDI-TOF mass spectra of the reaction product that results from the reaction of PNA with a 10 kDa PEG as shown on RP-HPLC (FIG. 21, 37.8 min). The product shows a broad mass ranging from approximately 18,500 Da to 20,200 Da. A broad peak is indicative of a polydisperse PEG.

### Mass Spec Analysis of 10 kDa PEG

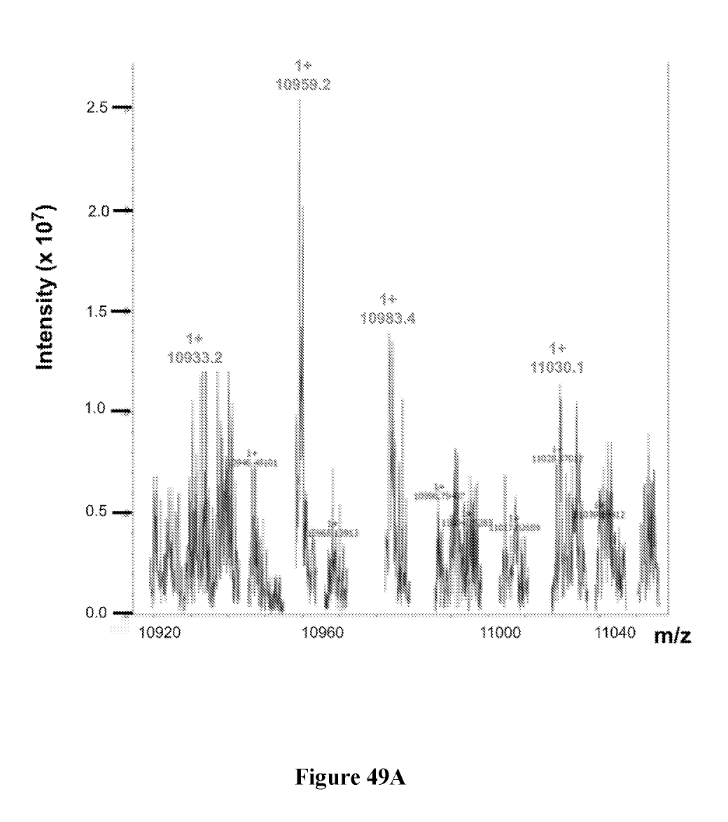

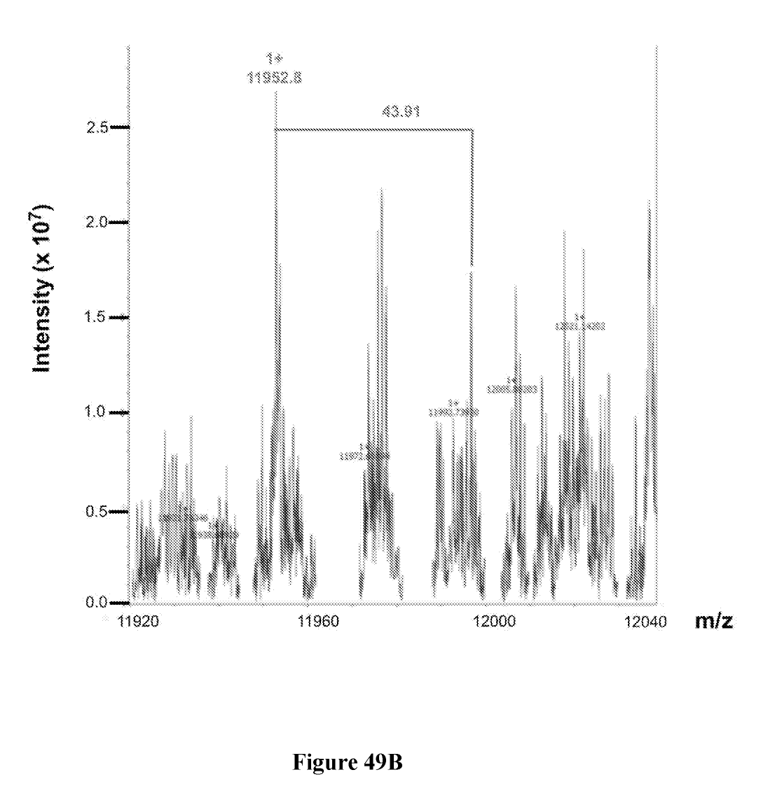

A solution of 50 uM maleimide-tagged 10 kDa PEG diluted directly in Ultrapure H2O was conducted on a Bruker Solarix FT-ICR-MS instrument in the positive ion mode. For analysis in positive mode, the instrument was calibrated utilizing Agilent's low concentration tune mix with 8 calibration points. The sample was diluted 1:1 with a solvent system consisting of 70:30 methanol:water (0.1% formic acid), and subsequently directly infused into the mass spectrometer. FIG. 49 shows the FT-ICR-MS profile of a maleimide-tagged 10 kDa PEG molecule. The mass to charge (m/z) ratio shows a polydisperse PEG molecule that has molecular weights ranging from 10920 (FIG. 49A) to 12040 (FIG. 49B). Specifically, FIG. 49A shows the mass profile ranging from 10920 to 11040. Multiple peaks are observed, with the strongest peak observed at 10959.2, indicating a high abundance of that species. FIG. 49B depicts the mass profile ranging from 11920 Da to 12040 Da, again exhibiting multiple peaks, indicative of a polydisperse molecule from the manufacturer. Mass separations of 43.91 units are observed (green), indicating the presence of differentially terminated PEG polymers. Because electrospray FT-ICR is a quantitative analytical method, the most abundant species of PEG is therefore found at 11952.8 and 10959.2. These results are in agreement with FIG. 23 which demonstrates a PNA-10 kDa conjugate mass ranging from 18858 to 19937 Da (PNA mass 7860 Da). Taken together, these data sets indicate successful conjugation and purification of the molecules used for downstream DNA invasion and nanopore analysis.

### Conjugation and Purification of the PNA-HIV Peptide Construct

A peptide previously found to be antigenic in the humoral immune response to HIV was first synthesized and purified by a commercial vendor (JPT Peptide Technologies). The peptide was created with a maleimide group to be used in subsequent coupling with the cysteine group of the PNA molecule. The sequence of the synthesized peptide product is as follows:

### Mpa-Ttds-KSIHIGPGRAFYTT

where Mpa is 3-Maleimido-propionic acid, Ttds is Trioxatridecan-succinamic acid that serves as a linker between the peptide antigen and the maleimide group, and the peptide sequence for the HIV antibody using standard 1-letter amino acid code.

This purified peptide product was sent to the PNA manufacturer (PNA Bio) for chemical coupling to the PNA molecule. Following conjugation, the PNA-peptide construct was purified, and characterized via mass spectrometry for use.

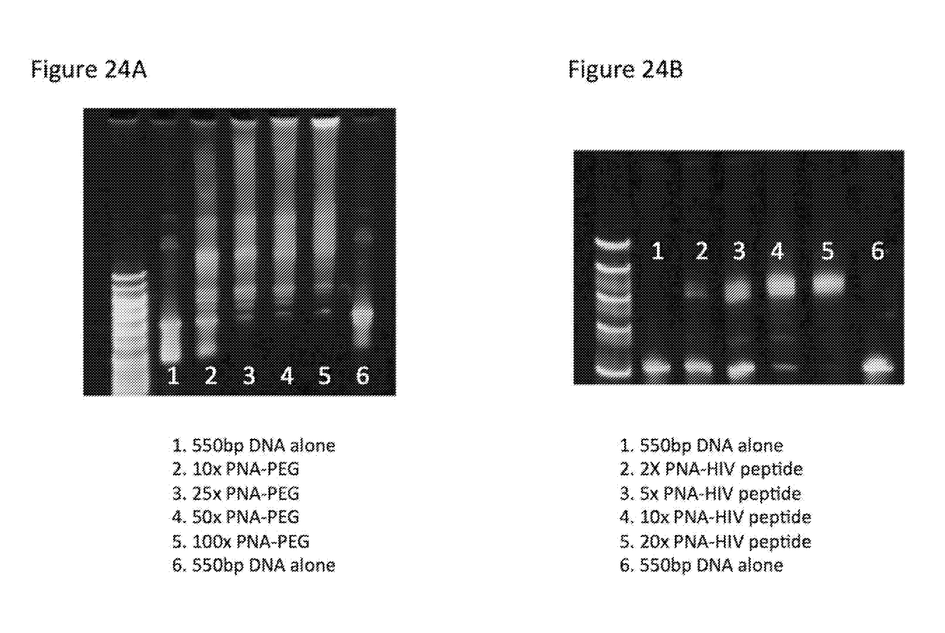

FIGS. 24A and 24B depict the gel shift assays between 550 bp DNA and purified PNA-PEG or PNA-HIV peptide conjugates. FIG. 24A demonstrates the invasion products of a 550 bp DNA fragment with an increasing concentration of purified PNA-PEG. Approximately 50% of DNA is bound by PNA-PEG at a 10-fold molar excess to DNA, and 100% of DNA is bound at a 25-fold molar excess. FIG. 24B shows the invasion capacity of PNA-HIV peptide onto a 550 bp DNA fragment. 100% of bare DNA is successfully invaded at a 10-fold molar excess of PNA-HIV peptide conjugate to DNA molecules.

Invasion of DNA by PNA-PEG on 3.25 kbp or 5.6 kbp dsDNA Fragments

Purified and rehydrated PNA-PEG was first titrated onto DNA to understand the DNA invasion (i.e., binding) capacity of the conjugate. 550 bp dsDNA containing a single PNA binding site was allowed to incubate with the PNA-PEG molecule for a period of 2 hrs at 60° C. (10 mM sodium phosphate, pH 7.4). To analyze the invasion products, the reactions were ran on a 5% TBE polyacrylamide precast gel (Biorad, Cat #456-5015) for 20 min at 100V and 35 min at 150V. This gel was later stained with 1×Sybr Green nucleic acid gel stain to visualize the relative change in the electrophoretic mobility of the reaction products, indicating to what extent the PNA-PEG conjugate had invaded the dsDNA scaffold (FIG. 24A).

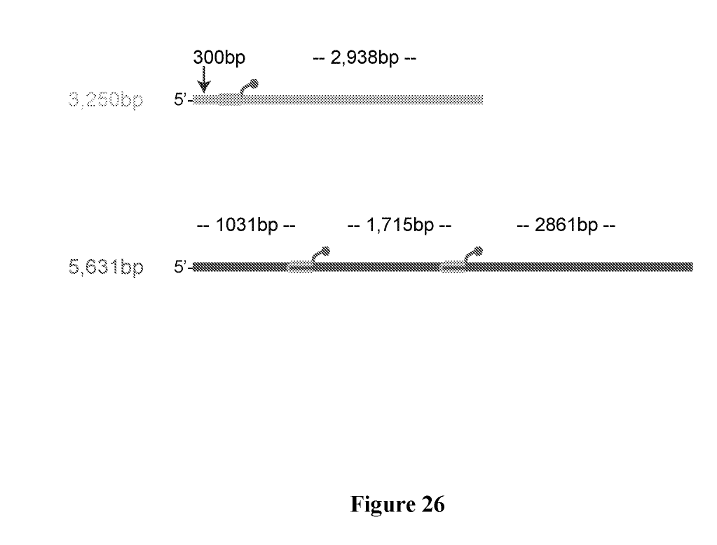

A 3.25 kbp dsDNA fragment containing a single binding site for PNA was then allowed to incubate with a 25-fold excess of PNA relative to dsDNA fragments for a period of 2 hrs 60° C. (10 mM sodium phosphate, pH 7.4). This concentration of molecular conjugate had previously been determined to invade 100% of free 550 bp dsDNA in solution with a single binding site. Following invasion, all samples were cleaned up of excess PNA-PEG probe by centrifugation using a 50 kDa filter (Millipore, Cat #UFC505024) for subsequent nanopore analysis. The 12 bp PNA binding site was located 300 bp from the 5′ end of the 3250 bp DNA (FIG. 26).

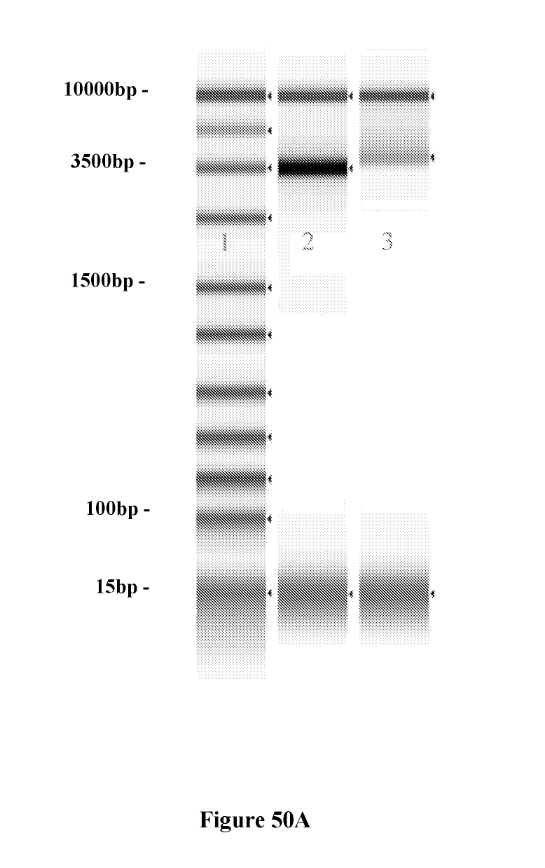

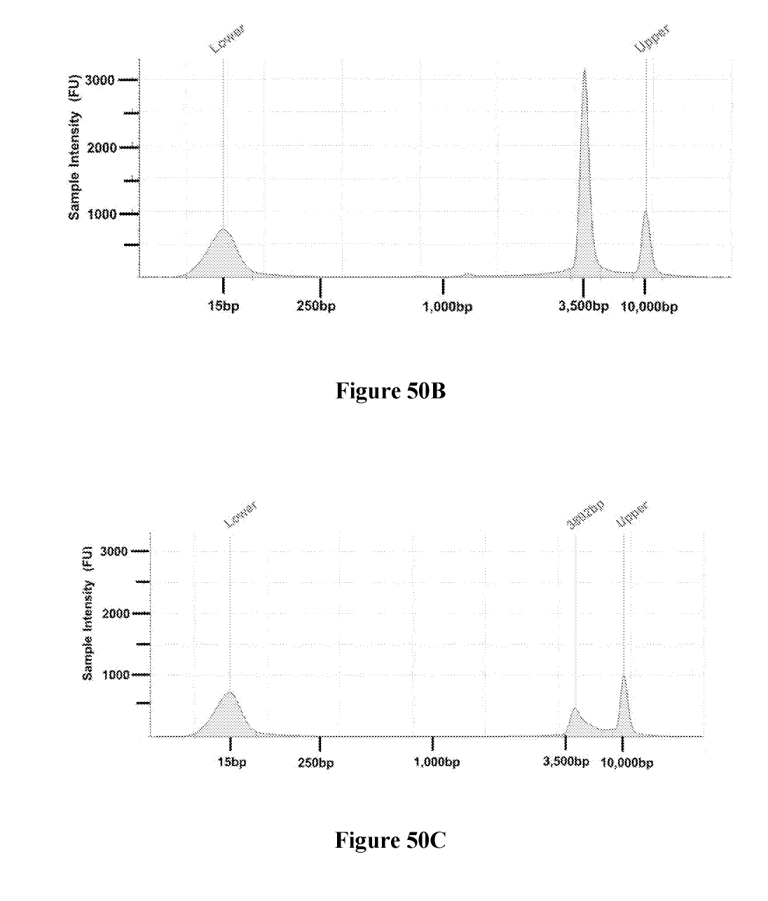

In order to verify PNA-PEG invasion onto the 3.25 kbp dsDNA fragment, samples were ran on a Tapestation 2200 (Agilent) using a High Sensitivity D5000 ScreenTape per manufacturer's instructions. Samples analyzed were diluted to 0.55 ng/4 μL. Invasion was assessed by a shift upwards in the gel matrix (FIG. 50). Specifically, FIG. 50A shows non-invaded 3250 bp DNA (Lane 2) that runs at the 3500 bp MW marker (Lane 1). Following invasion by the purified PNA-PEG molecule, an upward shift in the mobility of the molecule is observed (Lane 3), indicating successfully bound 3250 bp DNA fragment. FIG. 50B is a densitometry calculation of FIG. 50A Lane 2, demonstrating the intense band at 3500 bp. FIG. 50C shows the shift in band intensity as calculated by densitometry from 3500 bp to a new band at 3892 bp, indicative of complete DNA invasion by PNA-PEG.

A 5.6 kbp dsDNA fragment containing two binding sites for PNA was then allowed to incubate with a 10 or 25-fold excess of PNA relative to the number of DNA binding sites on the fragment. These concentrations of molecular conjugate had previously been determined to invade ˜50% and 100% of free 550 bp of DNA in solution, respectively, therefore creating a scaffold with 1 or 2 PNA-PEG molecules per dsDNA fragment. Following invasion, all samples were cleaned up of excess PNA-PEG probe by centrifugation using a 50 kDa filter and then submitted to nanopore analysis. The two 12 bp PNA binding sites were located 103 lbp and 2758 bp from the 5′ end of the 5631 bp DNA (FIG. 26).

### Invasion of DNA by PNA-HIV-Peptide Conjugate and HIV Antibody Binding

To characterize the DNA invasion capacity of the conjugate, a 550 bp dsDNA fragment was allowed to incubate with purified PNA-HIV conjugate at a 0 to 20-fold molar excess under the same buffer conditions as described previously. DNA invasion by the PNA-HIV conjugate were then visualized via an EMSA assay as before (FIG. 24B).

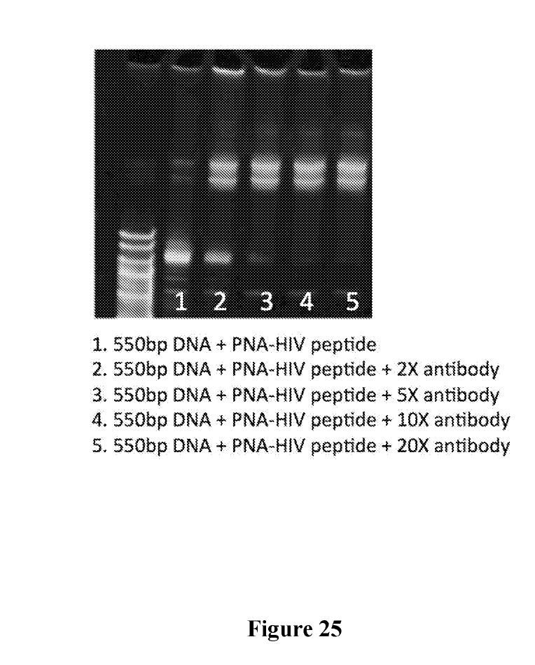

Following gel visualization to assess the best molar ratio of PNA-HIV peptide needed to successfully invade DNA, a large aliquot of the invasion product was produced and subsequently cleaned up of excess PNA-HIV peptide using a 50 kDa centrifugation filter. Antibody was then titrated into clean DNA-PNA-HIV peptide bait reagent at a 0 to 10-fold molar excess. The antibody bound DNA-PNA-HIV peptide was then visualized by an EMSA assay as before in order to determine the binding capacity of the antibody. Gel visualization showed that 2× antibody bound ˜50% of bait reagent while 5-fold excess was sufficient to bind all DNA-PNA-HIV peptide in solution (FIG. 25).

### Formation of a 2-Site 5.6 Kbp Scaffold Containing 1 PNA-PEG and 1 PNA-HIV Peptide Bound by HIV

A 5.6 kbp dsDNA fragment containing two sites for PNA invasion was first allowed to incubate with a 10-fold molar excess of HPLC purified PNA-PEG as previously described. This molar ratio was previously determined to bind ˜50% of all free DNA binding sites in solution according to a 550 bp EMSA assay. Once the PNA-PEG had been allowed to invade the 5.6 kbp fragment, it was cleaned up of excess PNA-peptide using a 50 kDa centrifugal filter unit. Following the cleanup process, the 5.6 kbp fragment containing ˜50% of binding sites occupied with PNA-PEG was allowed to incubate with a 10-fold excess of PNA-HIV peptide to invade all remaining PNA sites. All excess PNA-HIV peptide was again cleaned up as before.

Next, a 2-fold excess of antibody was allowed to incubate at room temperature for a period of one hour with the 5.6 kbp scaffold containing a site bound with PNA-PEG and the other bound with PNA-peptide (10 mM sodium phosphate, pH 7.4). This reaction product was then tested with a nanopore.

### Example 8—Multi-Level Detection Method Applied to 3250 bp DNA Scaffold without and with a Single PNA-PEG Payload Bound

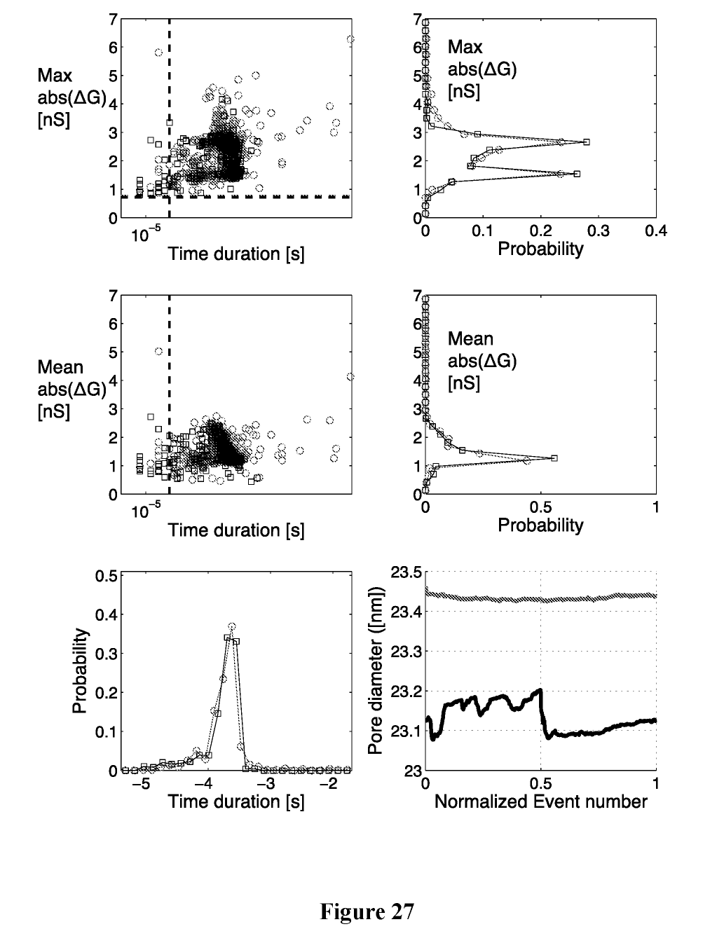

FIG. 27 shows the event plots for 3250 bp DNA without (black squares) and with (blue circles) a single PNA-PEG (10 kDa) bound to the DNA. The DNA-PNA-PEG complexes were formed as described in Example 7. Each event is represented by the maximum of the absolute value of the conductance shift (max abs(ΔG)) vs. duration in the upper left plot, and by the mean of the absolute value of the conductance shift (mean abs(ΔG)) vs. duration in the middle left plot. Conductance shift ΔG is the current shift ΔI (detected as the continuous samples below six times the signal standard deviation) for each event divided by voltage V. FIG. 27 also shows shift histograms (max and mean abs(ΔG)) and a duration histogram.

The DNA alone was measured first, following by the DNA-PNA-PEG reagent, with a nanopore 23.1-23.5 nm in estimated diameter (FIG. 27, lower right). The method of nanopore diameter estimation employed here and in other examples is presented in [Morin, Trevor J, et al., “Nanopore-Based Target Sequence Detection.” Edited by Meni Wanunu. PloS One 11, no. 5 (May 5, 2016): e0154426-21].

The DNA-PNA-PEG at 0.38 nM produced 594 events in 10 minutes, while the DNA alone at 0.38 nM produced 490 events in 10 minutes. The two populations looked largely indistinguishable. Specifically, the max abs(ΔG), mean abs(ΔG) and duration histograms show little difference between the event populations, with the exception of the slight increase in the fraction of max ΔG values larger than 3 nS with the PNA-PEG bound to the DNA.

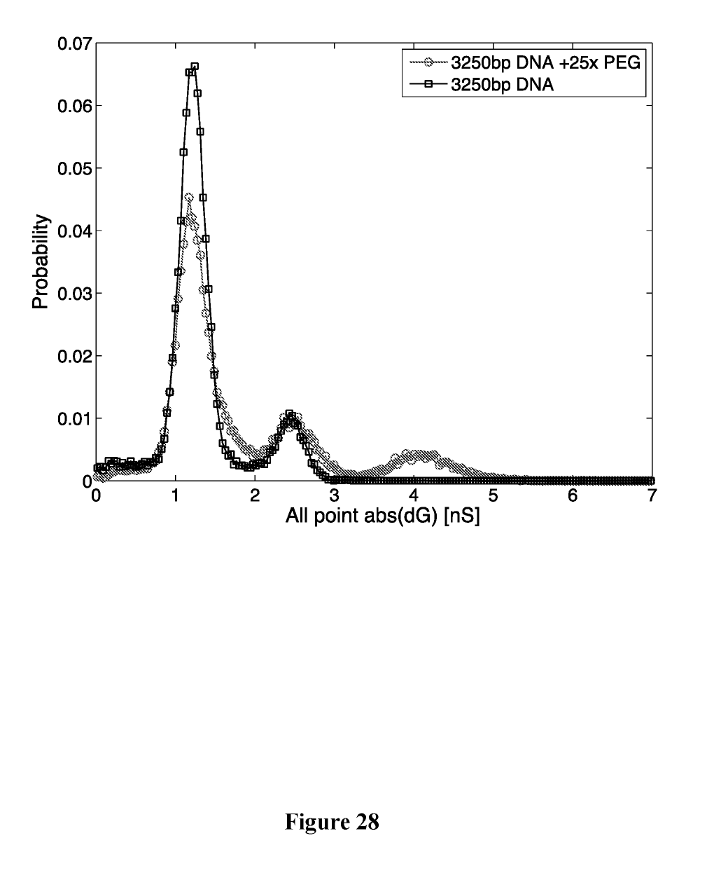

To compare the populations further, FIG. 28 shows an all point histogram, using every sample (absolute value) from every event in the histogram. The histogram shows that an appreciable fraction of the DNA-PNA-PEG molecules spend time at the 3.5-5 nS depth below the baseline, while DNA molecules do not.

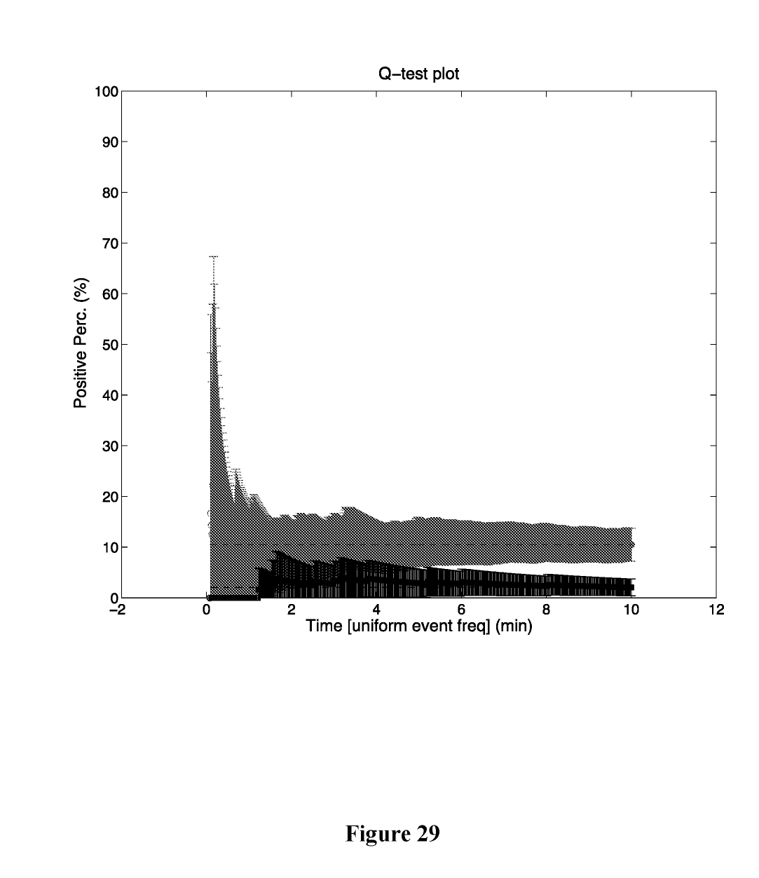

As another comparison, FIG. 29 shows the percentage of events with max abs(ΔG)>3 nS. The fraction and error bars are computed using the method presented in [Morin, Trevor J, et al., “Nanopore-Based Target Sequence Detection.” PloS One 11, no. 5 (May 5, 2016): e0154426-21]. The DNA-PNA-PEG reagents produces a final Q=10.4377+/−3.2314 at 99% confidence, while the DNA alone has Q(end)=2.0534+/−1.6553 at 99% confidence. As a result, positive detection of PNA-PEG-bound DNA is accomplished with 99% confidence, with a margin of ˜8% in payload-positive events using this metric.

### Multi-Level Detection Method

For the purpose of identifying a payload, in this case PNA-PEG, rather than looking at the grosser event features (max or mean conductance depth vs. total event duration), we apply step detection algorithms to look for step-like transitions within events that signal the presence of the payload (i.e., a label or detectable tag). Since the payload is bound near the end of the 3250 bp DNA, the presence of a “spike” or “bump” or “step” near either end of an otherwise “DNA event level(s)” would be expected for payload-bound DNA event.

Step detection methods are known in the nanopore literature, for example: [Pedone, Daniel, Matthias Firnkes, and Ulrich Rant. “Data Analysis of Translocation Events in Nanopore Experiments.” Analytical Chemistry 81, no. 23 (Dec. 1, 2009): 9689-94. doi:10.1021/ac901877z.], [Plesa, Calin, and Cees Dekker. “Data Analysis Methods for Solid-State Nanopores.” Nanotechnology 26, no. 8 (Jan. 29, 2015): 1-8. doi:10.1088/0957-4484/26/8/084003], and [Raillon, C, P Granjon, M Graf, L J Steinbock, and A Radenovic. “Fast and Automatic Processing of Multi-Level Events in Nanopore Translocation Experiments.” Nanoscale 4, no. 16 (Aug. 21, 2012): 4916-24. doi:10.1039/c2nr30951c.]. These methods attempt to approximate and reconstruct the signal as a pulse-train. As part of these methods, additional linear filtering (e.g., a moving average) is applied to the measured signal. A preferred method is to use a Savitzky-Golay Filter because, when they are appropriately designed to match the waveform of an oversampled signal corrupted by noise, they tend to preserve the width and height of peaks in the signal waveform [e.g., Schafer, Ronald. “What Is a Savitzky-Golay Filter? [Lecture Notes].” IEEE Signal Processing Magazine 28, no. 4 (Jun. 8, 2015): 111-17. doi:10.1109/MSP.2011.941097].

Alternatively, optimization-based methods (e.g., as in [Little, M A, and N S Jones. “Generalized Methods and Solvers for Noise Removal From Piecewise Constant Signals. II. New Methods.” Proceedings of the Royal Society a: Mathematical, Physical and Engineering Sciences 467, no. 2135 (Sep. 27, 2011): 3115-40. doi:10.1098/rspa.2010.0674.]) can be used to reconstruct a noiseless pulse-train approximation. More broadly, such an approximation can be generated by using linear programming, quadratic programming, semi-definite programming or nonlinear optimization methods (e.g., collocation).

We implement a combination of these methods in these examples. Specifically, sampled finite difference of a moving average is used to find inflection points (i.e., transitions between levels); subsequently, between pairs of inflection points the signal is averaged to estimate the level depth, and the time between inflections estimates the level duration. When more than 7 levels are identified within an event using this method, the method is reapplied to the signal after applying a de-noising algorithm (Proceedings of the Royal Society a: Mathematical, Physical and Engineering Sciences 467, no. 2135 (Sep. 27, 2011): 3115-40.) The de-noising algorithm performs robust discrete total variation denoising (TVD) using interior-point linear programming.

### Applying Multi-Level Detection Method to DNA Alone

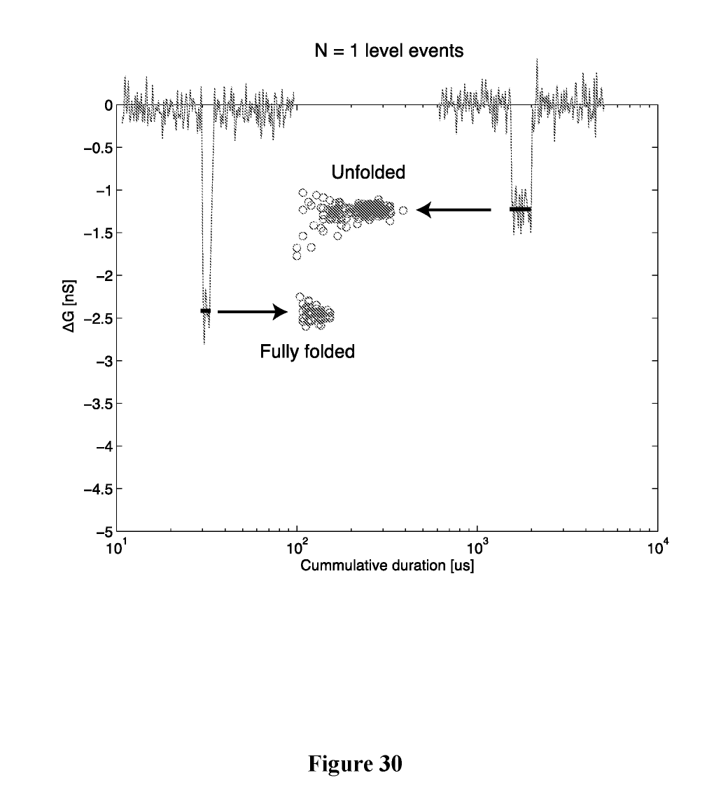

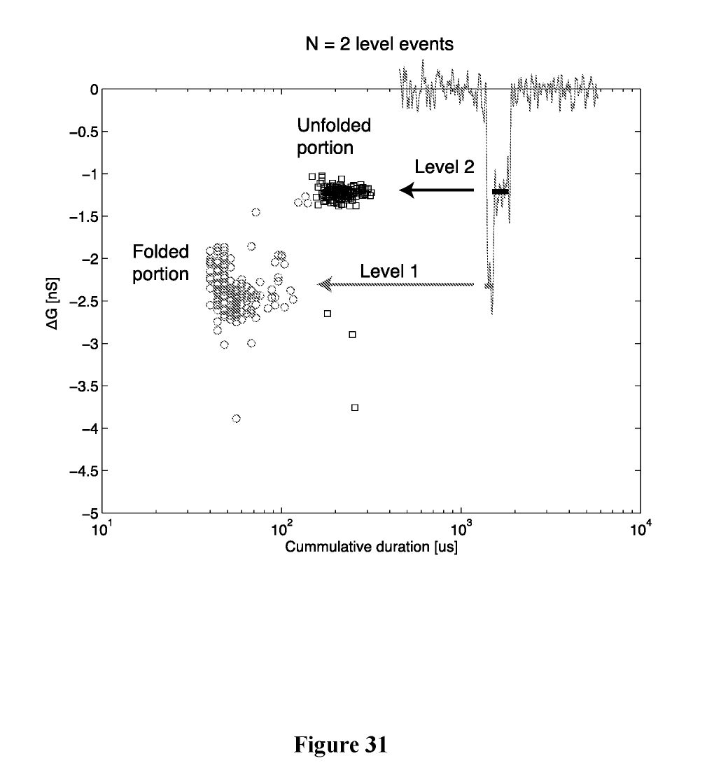

Applying this method to the 3250 bp DNA alone data set, events faster than 100 us were removed, as such evens represented non-passage collisions with the pore. Out of 410 counted events, events with 1 level are 59.7561% (245), events with 2 levels are 39.0244% (160), and events with 3 levels are 1.2195% (5). As shown in FIG. 30, level 1 events are made up of fully folded and unfolded DNA events. Fully folded events (approximately −2.4 nS depth and 100-200 μsec duration) are twice as deep and half as long as unfolded events (approximately −1.2 nS depth and 200-400 μsec duration). As shown in FIG. 31, level 2 events are made up of partially folded DNA events, in which the folded portion first passes through the pore. The presence of fully folded, partially folded and unfolded event types for DNA alone is well known in the literature (Storm, A, J Chen, H Zandbergen, and C Dekker. “Translocation of Double-Strand DNA Through a Silicon Oxide Nanopore” 71, no. 5 (May 2005): 051903. doi:10.1103/PhysRevE.71.051903.)

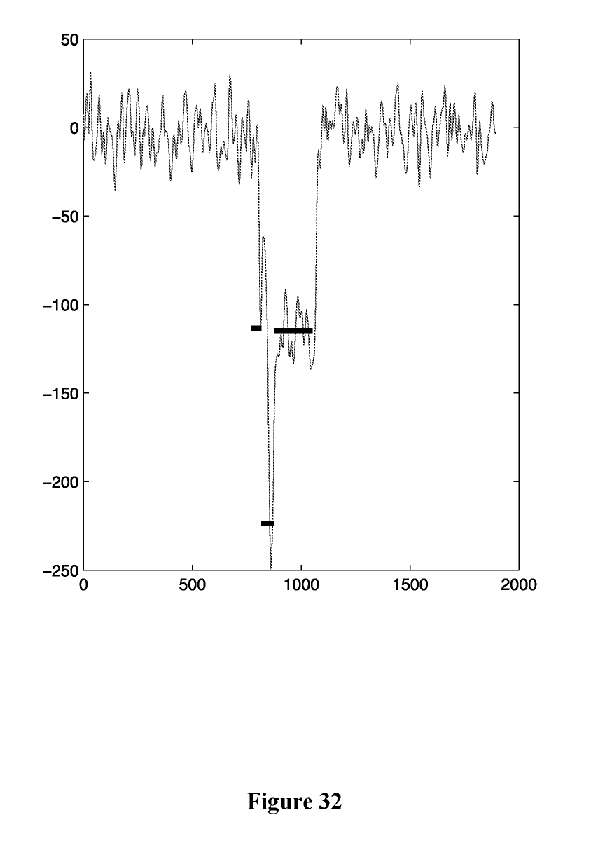

While 5 events out of 410 registered as having 3 levels, such events were consistent with partially folded DNA (FIG. 32), with an initial “shoulder” to the event. No events had more than 3 levels detected.

Applying Multi-Level Detection Method to DNA with PNA-PEG Reagent

We consider next the algorithms and analysis applied to the DNA-PNA-PEG data set. After events faster than 100 us were removed, 511 events remained in the DNA-PNA-PEG data set.

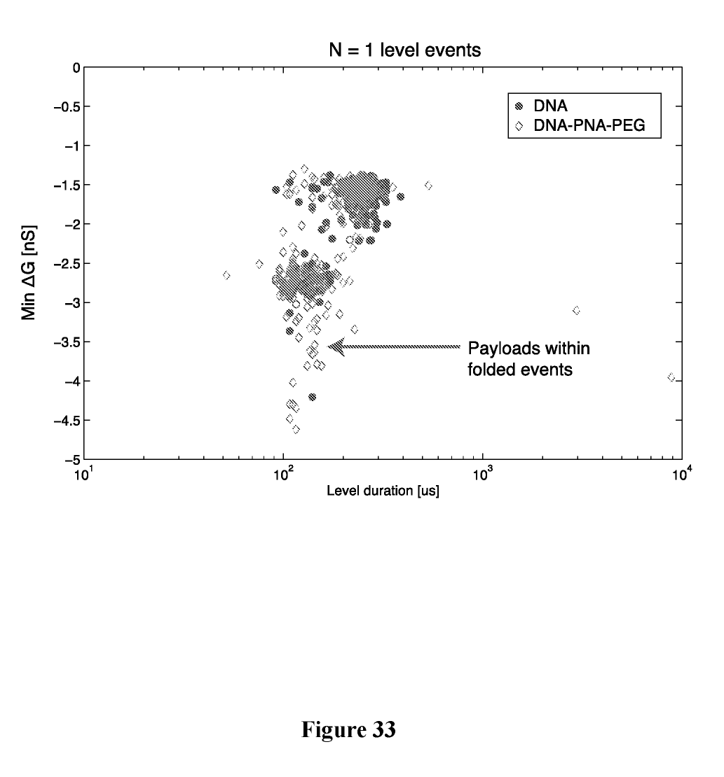

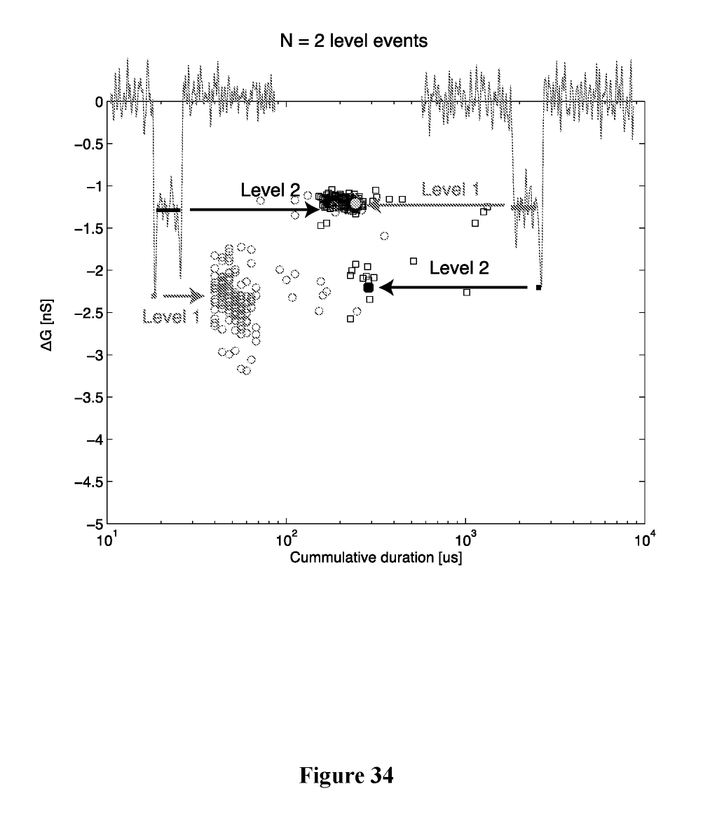

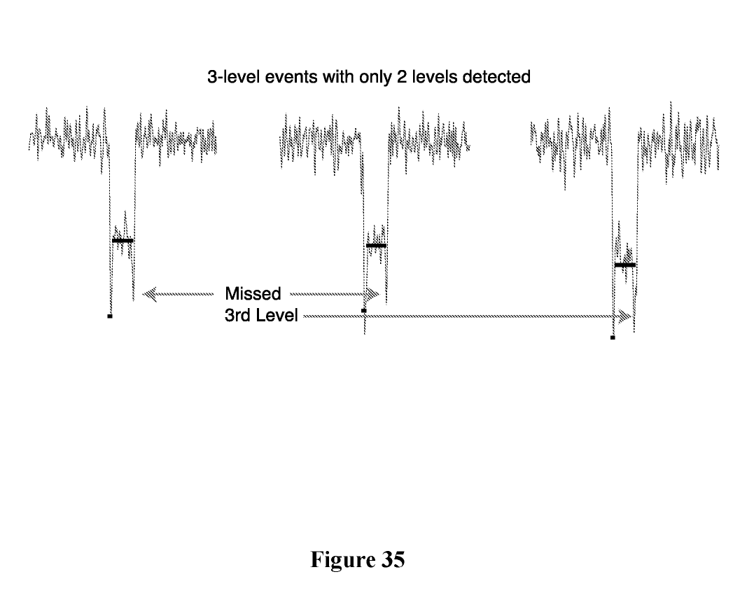

FIG. 33 compares the minimum sample within N=1 level events for the 3250 bp DNA alone experiment and for the 3250 bp DNA with PNA-PEG experiment. By plotting the minimum sample, instead of the fitted level (and average), we can test for the presence of deeper “spikes” that would signal that a payload-bound DNA passed through the pore in an otherwise fully folded or unfolded state. Of fully folded events (below −2.3 nS), only 5% of events for DNA alone were below −3 nS, while 25% of 132 folded events in the DNA-PNA-PEG experiment were below −3 nS. These deeper samples within fully folded events are attributed to the payload bound to DNA. Due to the temporal resolution limits of the sensor, an appreciable number of fully folded events with payloads would not show a detectable “spike”; this can be resolved by improvements in the amplifier, and will also improve for longer DNA that shows a longer lasting fully-folded event pattern. For the unfolded events (between −1 and −2.3 nS), a deeper spike was not readily observed; this is due to such events more commonly being identified as having two levels, a first level at the unfolded DNA depth, and a second deeper (“spike”) level signaling the presence of the payload. An example of this is shown in FIG. 34, with the representative DNA-PNA-PEG event has a level 1 depth of −121.6883 pA and duration 224 us (consistent with unfolded DNA) followed by level 2 depth of −209.514 pA and duration 44 us. Such events were not present in the DNA alone 2-level event plot (FIG. 31). In addition, a fraction of the 2-level events showed a third-level visually that was not detected (FIGS. 34 and 35), with the terminal “spike” again signaling payload-bound DNA since such spikes did not occur in the DNA alone control.

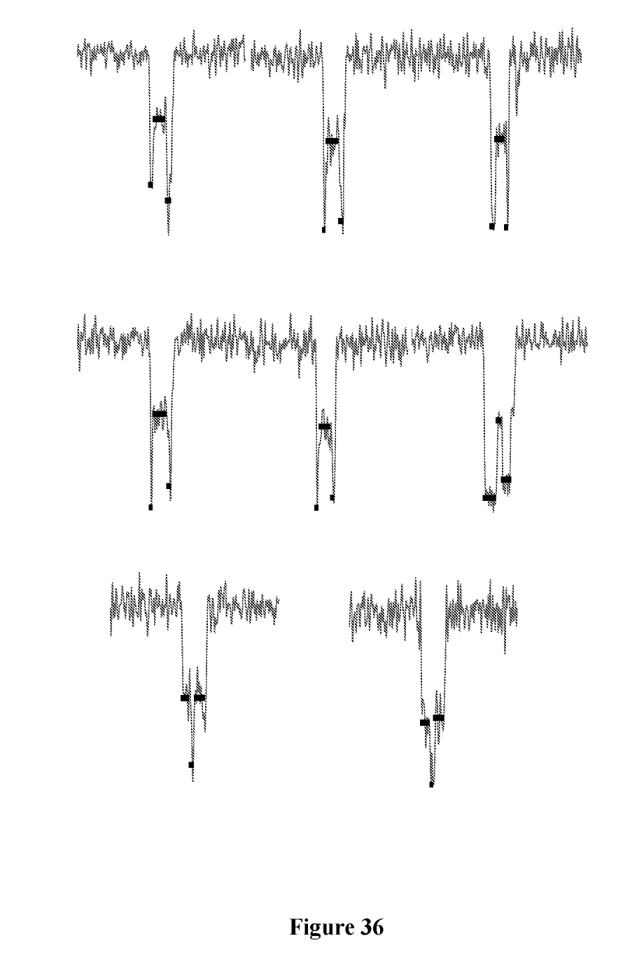

The algorithm identified 9% (46) of the 511 as having 3 or more levels, which is considerably less than the fraction (30-50%) that visually showed three or more levels. Specifically, Level 1 is 57.3386% (293), Level 2 is 33.6595% (172), Level 3 is 5.8708% (30), Level 4 is 1.5656% (8), Level 5 is 0.97847% (5) and Level 6 is 0.58708% (3). Refinements in the algorithm can be made to reduce these missed 3rd levels (FIG. 35), though for the purpose of illustrating the methods of this patent, the performance of the algorithm is sufficient. As stated, improvements in the temporal resolution of the measurement circuitry can increase the detectable fraction that otherwise pass through the pore too fast for payload (“spike”) detection. FIG. 36 shows some of the representative N=3 level events from the DNA-PNA-PEG reagent experiment, with the first two rows showing the down-mid-down signature pattern consistent with a partial fold—unfolded—payload-end translocation pattern. The third row shows a few events with a less frequent (mid-down-mid) pattern.

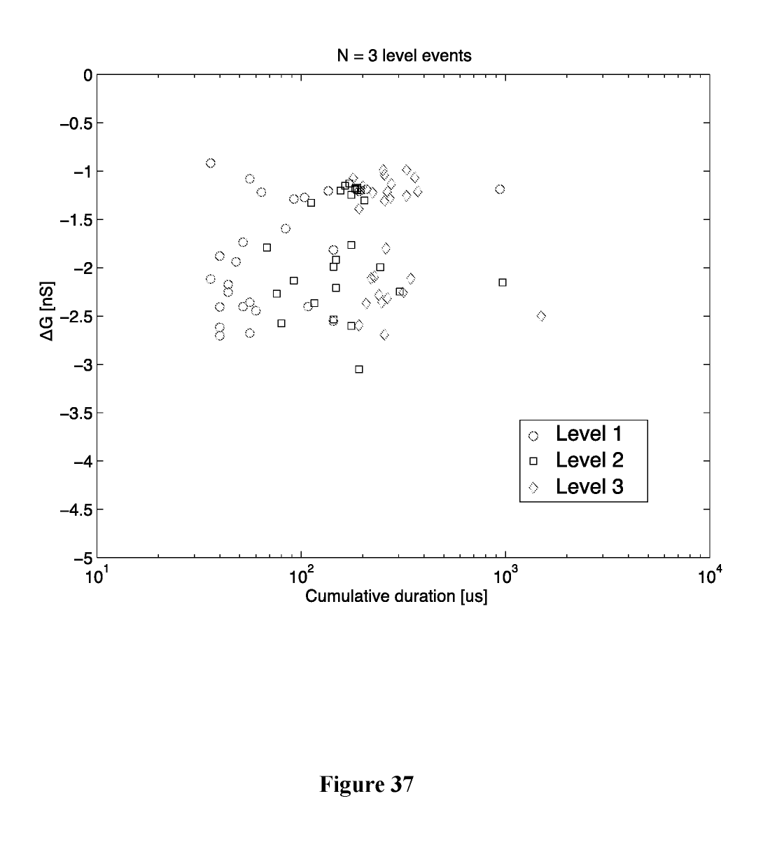

All N=3 level identified events from the DNA-PNA-PEG reagent experiment are shown in FIG. 37, with levels demarked by the first level as a blue circle, second level as a black square, and third level as a red diamond. Depth of each level is plotted vs. cumulative duration, i.e., the second level depth is plotted vs. the sum of the first level and second level duration, and the third level depth is plotted vs. the sum of all three level durations. By plotting cumulative duration, the relative horizontal location of the level points can be visually matched to event patterns (e.g., FIG. 36).

In summary, ˜25% of events from the DNA-PNA-PEG reagent experiment displayed a pattern that signals the presence of the payload. Improvements in the measurement circuitry can boost this percentage further. Other methods include making the scaffold longer, and also increasing the size and the number of adjacent payloads on the molecule to increase the likelihood of positive payload detections, even with existing circuitry. By using a level-detection algorithm, even without optimizing the performance of the algorithm, the percentage of payload-positive events increased dramatically (25%) compared to the standard of using a single-level approximation for all events (i.e., relying on max abs(ΔG) or mean abs(ΔG)) which showed only an 8% difference between the samples without and with the payload. Since the payload is near the end of the molecule in this example, we next sought to examine the performance of having 1 and 2 payloads in a longer DNA (5.6 kb) and not near the ends (one 1031 bp from 5′, and the other 2861 bp from the 3′, FIG. 26).

### Example 9—Multi-Level Detection Method Applied to 5631 bp DNA Scaffold without and with Up to Two PNA-PEG Payloads Bound

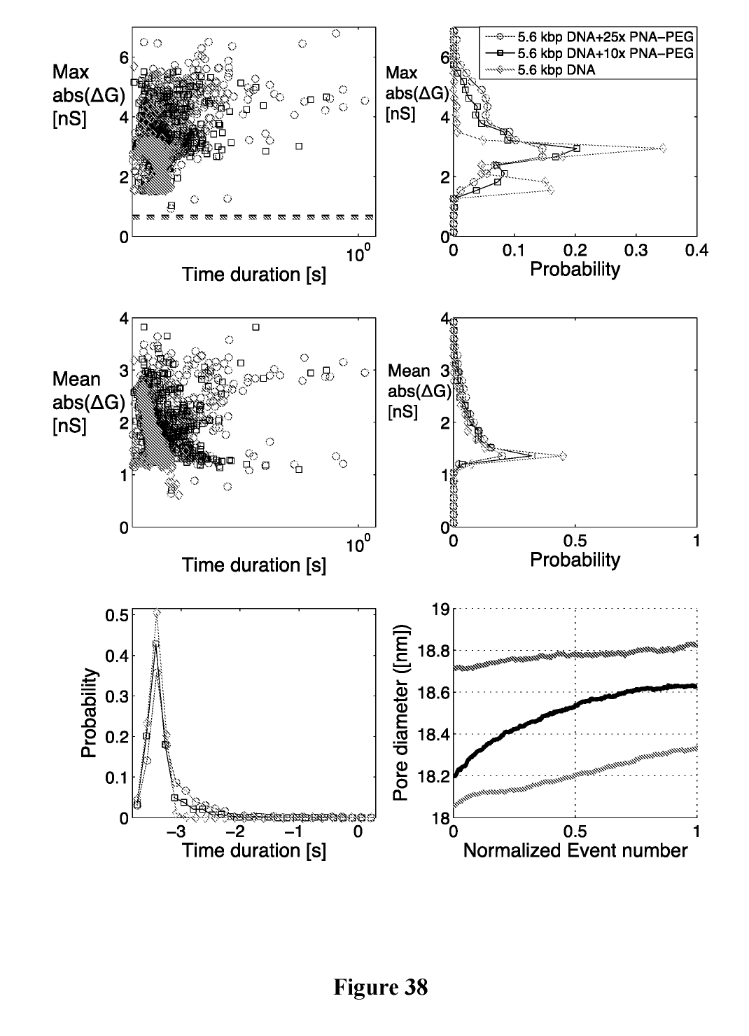

FIG. 38 shows the event plots and histograms for 5631 bp DNA without (red diamonds) and with PNA-PEG (10 kDa) at 10× (black squares) and 25× (blue circles) the number of sites (2 sites per DNA, FIG. 26). The reagents were sequentially tested on the same 18-19 nm diameter pore.

The 5.6 kb DNA alone at 0.3 nM produced 738 events in 21 minutes, followed by 0.3 nM DNA with 10× (6 nM=2×3 nM per site) PNA-PEG (10 kDa) producing 715 events in 21 minutes, and subsequently 0.3 nM DNA with 25× (15 nM) PNA-PEG (10 kDa) producing 753 events in 35 minutes. As a final control, 25×PNA-PEG (15 nM) alone was run, producing only 9 events in 12 minutes.

The three populations appear distinguishable, with deeper and longer lasting events more likely proceeding from 25×PNA-PEG to 10×PNA-PEG to no PNA-PEG.

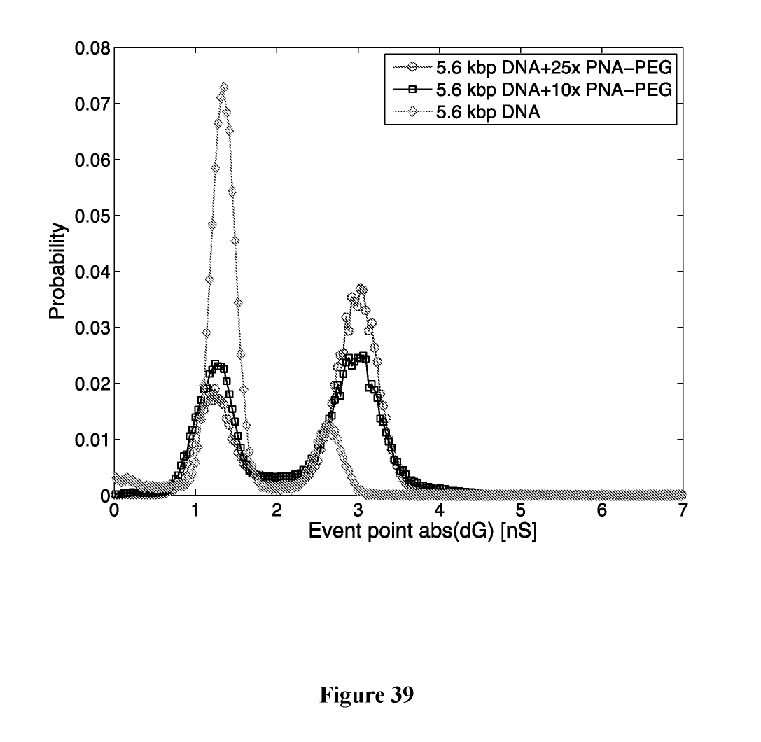

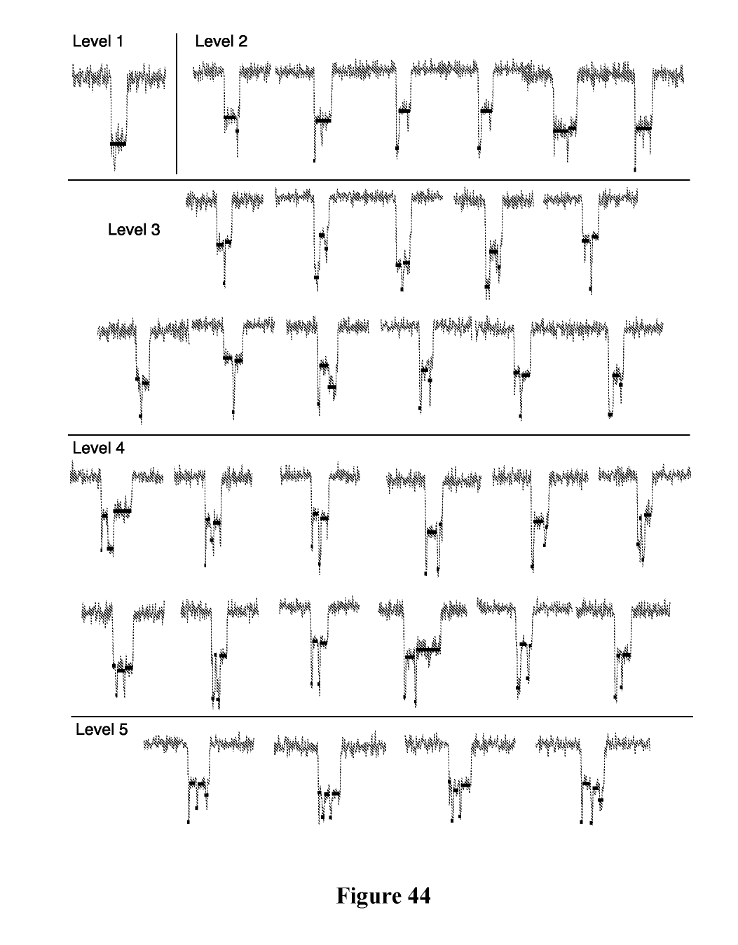

To compare the populations further, FIG. 39 shows an all point histogram. The histogram shows that an appreciable fraction of the DNA-PNA-PEG molecules spend time at the 3-5.5 nS depth below the baseline, while DNA molecules do not. The DNA alone peaks correspond to unfolded DNA (red, 1.35 nS peak) and folded DNA (red, 2.7 nS). While the unfolded DNA level is conserved for the events with DNA and DNA-PNA-PEG, the second peak for DNA-PNA-PEG is wider and taller (with a max at ˜3 nS), suggesting the presence of the payload bound to unfolded DNA creates a depth that is slightly deeper than folded DNA alone. The signal difference between unfolded DNA and the unfolded DNA-payload (3 nS−1.35 nS=1.65 nS=165 pA at 100 mV) is large enough (SNR>5) to detect above the noise (−30 pA RMS at 30 kHz, and −15 pA RMS at 10 kHz), provided the feature remains in the pore long enough compared to the temporal limit of the instrument. Since the 5.6 kb has a 400 us mean duration, that's 4.7 nm/us, and for a 22 nm length pore, that's 5 us of transit time for the PNA portion of the PNA-PEG payload when bound to unfolded DNA. Since the 10 kDa PEG extends ˜72 nm (210 bp in length), the transit time of the PEG portion of the payload is 20 us. At 30 kHz, the time to resolve a pulse at full depth is 24 us (filter rise time is 12 us), suggesting that the payloads should be resolvable (albeit not at full depth) a significant portion of the time when passing through the pore bound to unfolded DNA. This is indeed the case, as described below and by examining representative events (FIG. 44). Since most payload “spikes” don't hit full depth, the peak at 3 nS in the all point histogram is likely shallower than the true depth that the payload bound to DNA would register, e.g., if the temporal resolution of the instrument were faster, of if the payload spent slightly more time in the pore (this can be achieved by lowering voltage, increasing salt, increasing pore length). It is important too to note that when DNA passes through the pore faster than average, the time the payload is in the sensing region will be less, and can go undetected by the algorithm and may even be visually not detectable.

In our scheme, the all point histograms can inform the choice of bin locations, with which detected levels within each event can be individually assigned to a corresponding state (i.e., unfolded bare dsDNA, payload-bound unfolded dsDNA, once folded bare DNA, payload-bound once folded DNA, twice folded bare DNA, etc.). From FIG. 39, for example, unfolded bare dsDNA is 0.7-2 nS, once folded bare DNA is 2-2.9 nS, payload-bound unfolded dsDNA is 2.9-4 nS, and payload-bound once folded DNA is levels below 4 nS. For 5.6 kb, more than one fold is unlikely, although more than one fold is more likely with DNA 10 kb and longer.

Observe also that the payload-bound peak (3 nS) is higher for 25×PNA-PEG than 10×PNA-PEG, suggesting that a higher percentage of molecules have a payload bound at both binding sites at 25× than at 10×, as expected from the gel assays (Example 7).

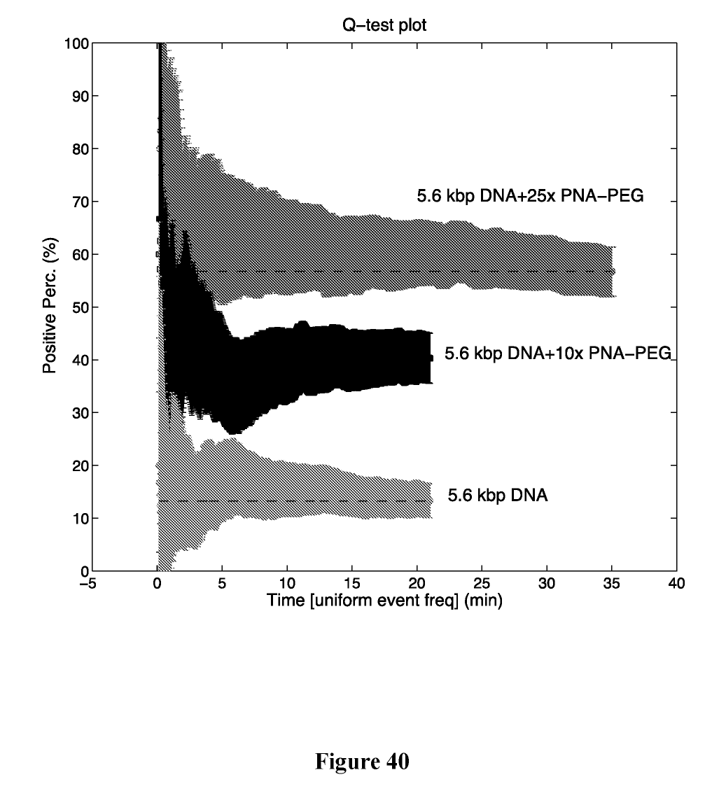

FIG. 40 shows the percentage of events with max abs(ΔG)>3 nS. The fraction and error bars are computed using the method cited in Example 8. The DNA alone produced a final Q=13.2791+/−3.2176, while the DNA with 10×PNA-PEG had Q=40.2797+/−4.7246 and the DNA with 25×PNA-PEG had Q=56.7065+/−4.651, reporting error at 99% confidence. As a result, positive detection of PNA-PEG-bound DNA is accomplished with 99% confidence, with a margin of ˜27% and ˜43% in payload-positive events using this metric. The increase in the fraction of events with a max sample deeper than 3 nS (absolute value) at 25× compared to 10× is a consequence of the higher number of molecules with both binding sites occupied by a PNA-PEG payload.

### Applying Multi-Level Detection Method

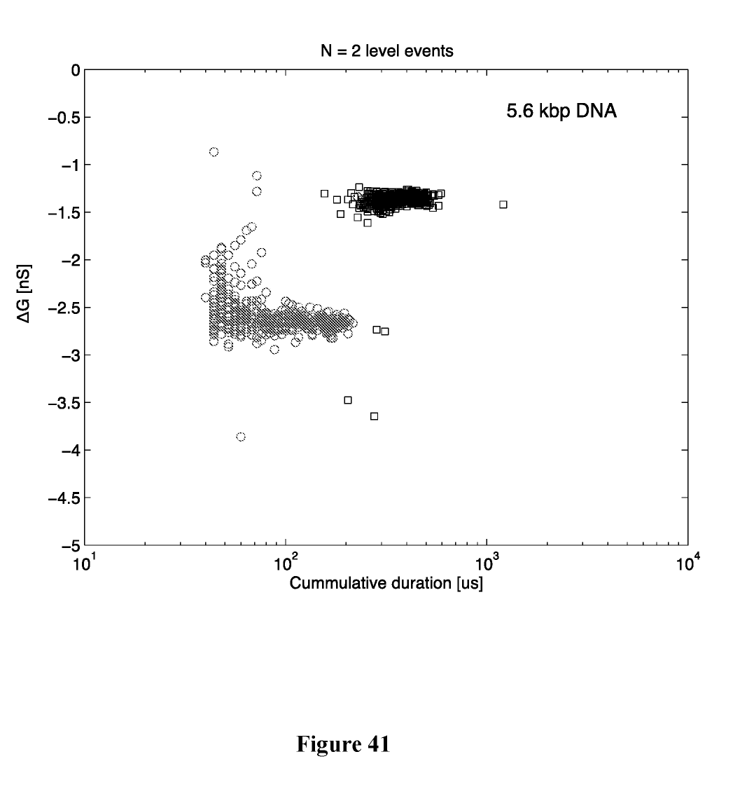

Applying the multi-level detection algorithm described in Example 8, the N=2 level events for 5.6 kb DNA alone are shown in FIG. 41 (plotting level depth vs. cumulative duration as before). As with the 3.2 kb DNA, capture in a partially folded state is the predominant 2-level mode of passage through the pore, with an expected observable increase in duration for each level of the 5.6 kb compared to the shorter 3.2 kb DNA (FIG. 31).

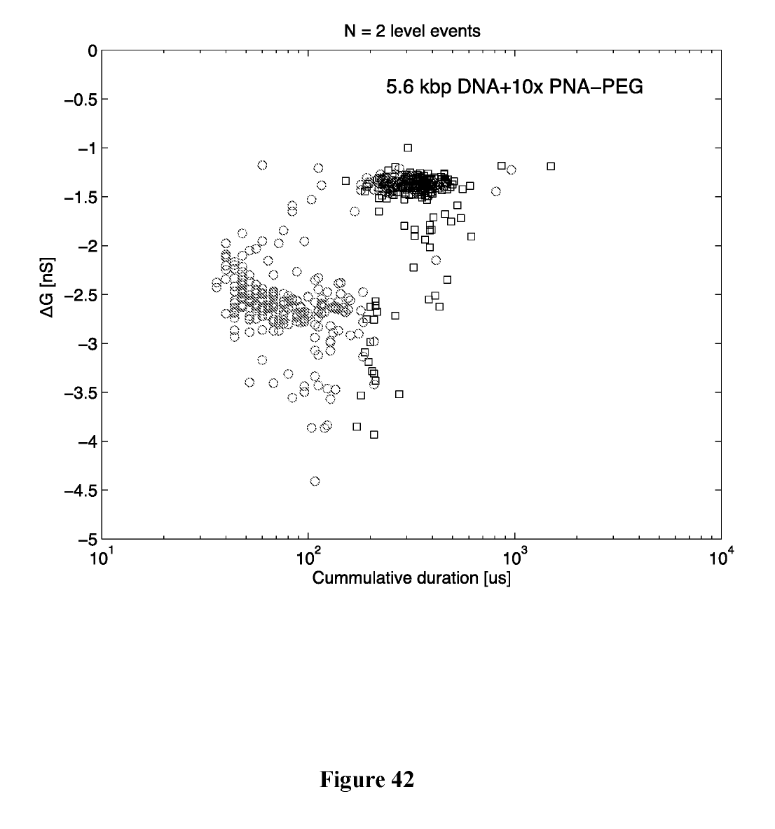

By contrast, the N=2 level events for DNA with 10×PNA-PEG (FIG. 42) show new subpopulations of events, some with a terminal step at the end of an otherwise fully folded event, and others with a terminal step within an otherwise unfolded event.

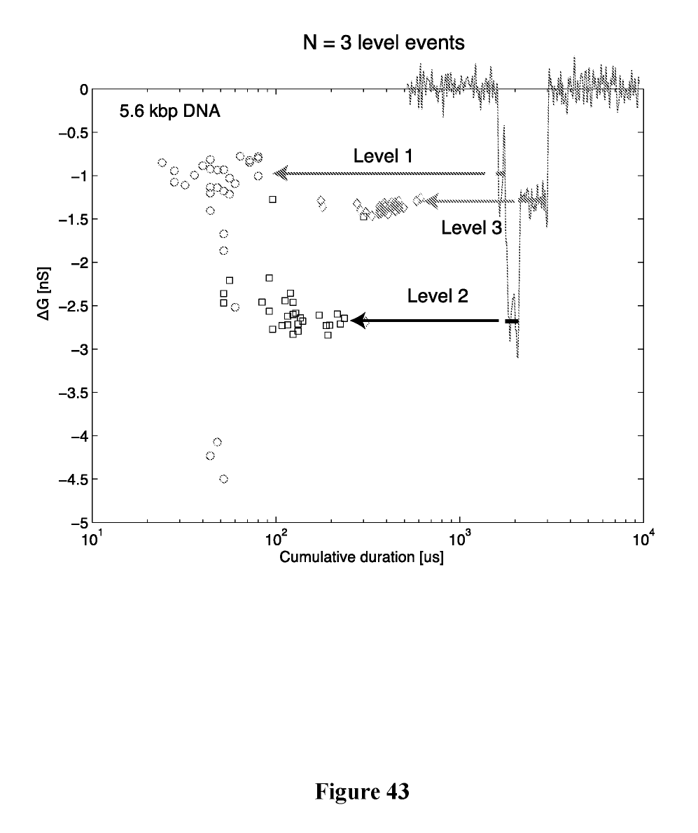

Out of 738 counted events for 5.6 kb DNA alone, the breakdown by number of levels detected are: Level 1 is 41.5989% (307), Level 2 is 53.3875% (394), Level 3 is 4.336% (32), and Level 4 is 0.67751% (5). FIG. 43 shows the rare N=3 level identified events from the 5.6 kb DNA experiment. The representative event shows that the payload depth is not present even in these 3-level events.

FIG. 44 shows representative events with N=1, 2, 3, 4 and 5 levels identified from the 5.6 kb DNA with 10×PNA-PEG experiment. Clearly, all events visually show a 1, 2 or 3 “spike” pattern, and the algorithm does not always detect each pattern faithfully. For example, there are shown two 5-level “mid-down-mid-down” patterns, where each “down” is a “spike” (i.e., a three “spike” pattern). The same pattern is visible in at least one of the N=4 and N=2 examples shown, which the algorithm missed. The same can be said for the two 5-level “mid-down-mid-down-mid” patterns (i.e., a two “spike” pattern), for which there are similar examples in the N=2, 3, 4 level cases. As stated, improvements in the algorithm and increasing the sensing time of the payload (e.g., by increasing size of payload, and/or the length of the pore/sensing region) will improve the “spike” detection performance.

The initial spike may be folded DNA, or may be a payload. When three spikes are registered, the initial spike can be ruled folded DNA, since there are only two payloads. When the initial spike is longer than an amount consistent with payload durations, it can also be ruled a folded DNA. Examples of this are the middle two events in the Level 2 cases shown in FIG. 44. This is not a perfect strategy, of course, since sometimes the payload event can be longer than usual (consider the first event shown in the Level 4 cases).

The middle and final spikes are attributable to having one or both payloads bound. Such features within events are not present in the DNA alone events. The algorithm does not perfectly detect all levels, as stated, but there is a correlation between having N=3 and a single payload, having N=5 and two payloads. Most of the N=4 appear attributable to single payloads also.

Since the 3.2 kb scaffold had a binding site for a single payload near (300 bp) the end of the molecule, the “spikes” almost universally occurred at the end of the event (a terminal “spike”). By contrast, the 5.6 kb scaffold has payloads more centrally located (FIG. 26), which is the reason for the “spikes” being more centrally located within events (FIG. 44).

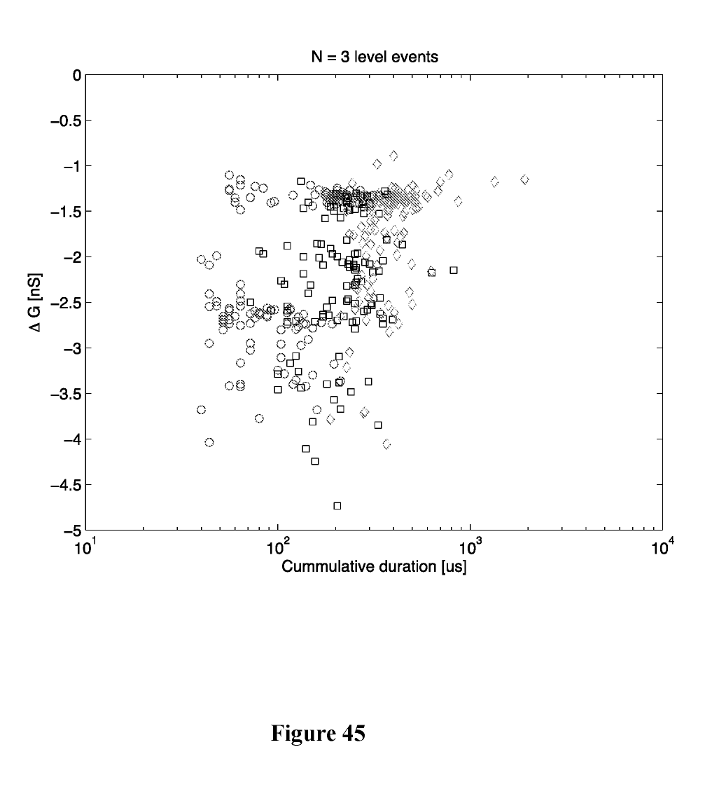

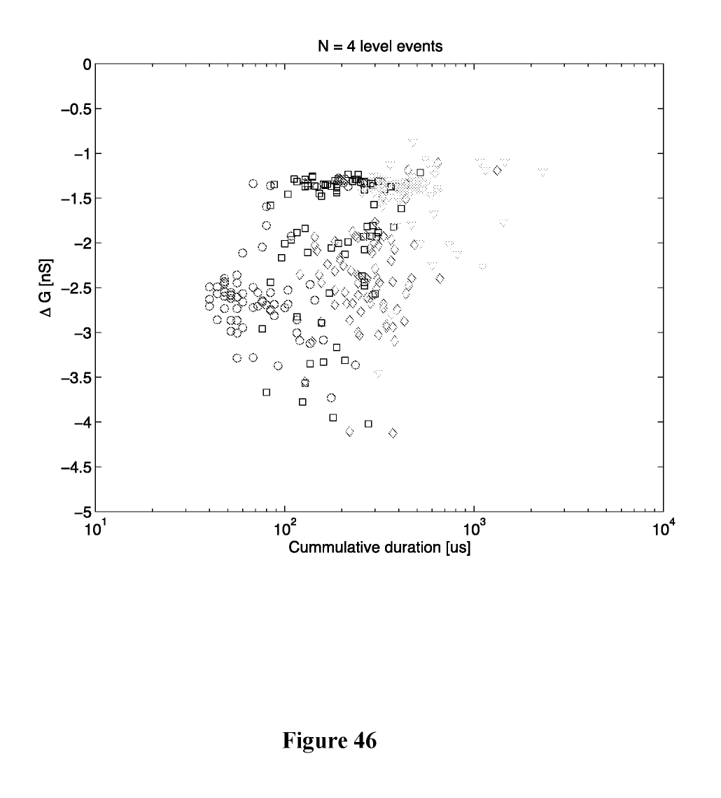

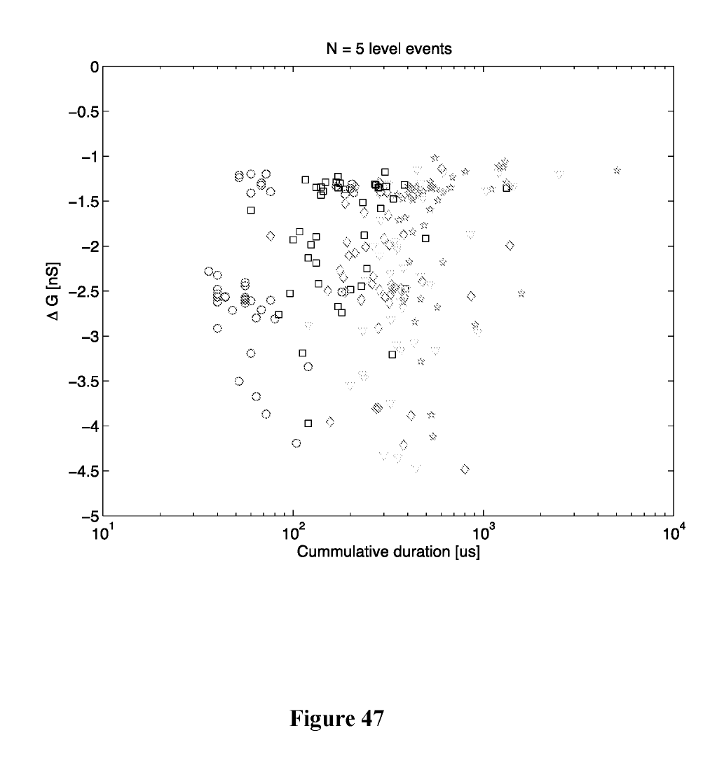

Out of 711 counted events for 5.6 kb DNA with 10×PNA-PEG, the breakdown by number of levels detected are: Level 1 is 20.5345% (146), Level 2 is 36.7089% (261), Level 3 is 19.2686% (137), Level 4 is 11.8143% (84), Level 5 is 6.1885% (44), Level 6 is 2.9536% (21) and Level 7 is 2.5316% (18). FIGS. 45, 46 and 47 shows all N=3, 4 and 5 level identified events, respectively, from the 5.6 kb DNA with 10×PNA-PEG experiment.

Out of 743 counted events for 5.6 kb DNA with 25×PNA-PEG, the breakdown by number of levels detected are: Level 1 is 14.6703% (109), Level 2 is 23.8223% (177), Level 3 is 21.6689% (161), Level 4 is 17.6312% (131), Level 5 is 11.8439% (88), Level 6 is 6.4603% (48), and Level 7 is 3.9031% (29).

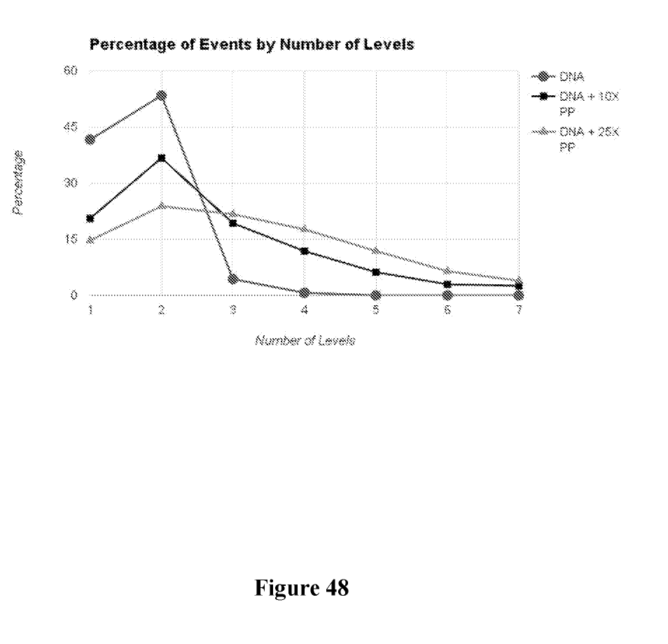

FIG. 48 shows the breakdown (by percentage) of the events by number of identified levels, for DNA alone, DNA with 10×PNA-PEG and DNA with 25×PNA-PEG data sets. In increasing the amount of PNA-PEG from 10× to 25×, the trend in FIG. 48 reflects the increase in the number of events with both sites on the DNA occupied with a PNA-PEG payload. The fraction of events with 3 or more levels is 5.0135%, 42.7566% and 61.5074% for DNA alone, DNA with 10× and 25×PNA-PEG, respectively. The margin of detection of at least one payload, and the ability to discriminate between 1 and 2 payloads, can be improved further still. For example, 3 level event patterns as shown in the DNA alone case (FIG. 43) can be excluded. Additionally, the 2-level event patterns showing a terminal spike can be included (FIG. 42). More broadly, improved detection of “spikes” within events is the most critical step toward improving the margin of detection. Discrimination of payload free, single payload, and double-payload scaffolds, as described herein, improves assays for target analyte detection, and for encoding information.

### Example 10—Multi-Level Detection Method Applied to 5631 bp DNA Scaffold with a PNA-PEG Payload and a PNA-Peptide for HIV Antibody Binding and Detection

HIV Ab Detection with Scaffold-Fusion (Payload Fee Case)

Before establishing that HIV Ab detection can be accomplishing using payload-bound scaffolds, we first show nanopore detection results using a scaffold-fusion without a payload.

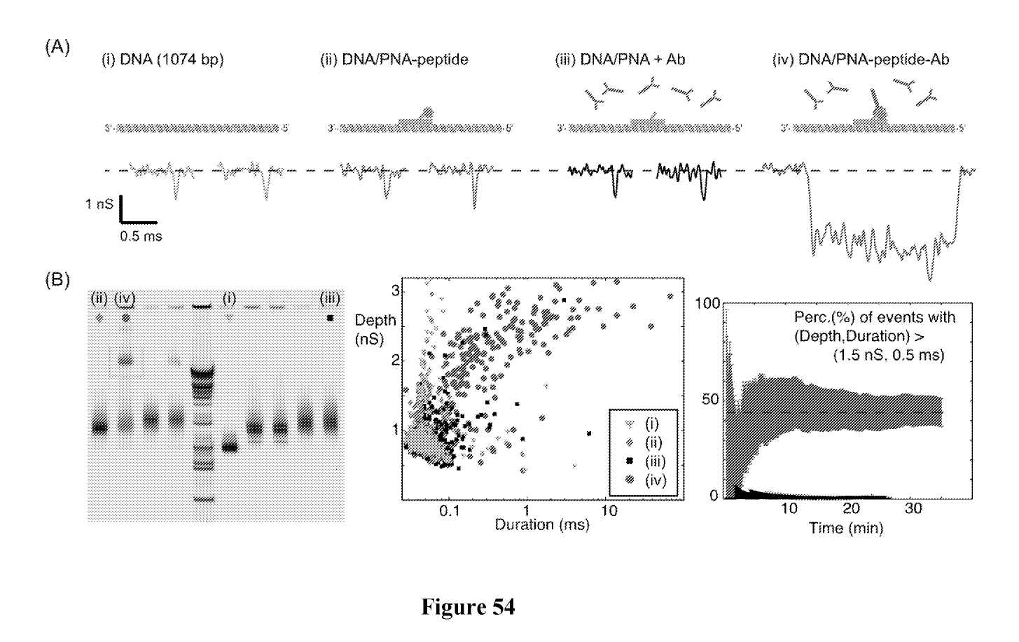

The fusion molecule links a bisPNA to a V3-loop peptide antigen (a maleimido-peptide that mimics the highly immunogenic V3 loop of the HIV Envelope (Env) protein for capturing genotype clade E HIV antibodies) and the HIV antibody show that the full complex (DNA/PNA-peptide-Antibody) alone gives a distinct nanopore event signature that can be discriminate this complex from all other background (FIG. 54). The scaffold in these results is a linear 1074 bp dsDNA molecule (Genewiz) that contains one bisPNA binding sequence (CCTTTCCCTTCC) positioned in the center of the molecule. The 12-mer bisPNA (24mer including anti- and parallel strands, PNABio) was chosen since this length is easily synthesized and is long enough to remain bound to the target dsDNA sequence even in high salt (1M LiCl). Each reagent shown in FIG. 54 (i-iv) was sequentially tested for 20-30 minutes, separated by event-free buffer only periods, using a 30 nm diameter pore (100 mV, 1M LiCl). In the absence of the peptide component of the fusion molecule (reagent iii), the DNA/PNA+HIV antibody reagent produced nanopore signatures indistinguishable from DNA/PNA (ii) and DNA (i) alone. A separate HIV antibody alone control (not shown) produced no events. When the reagent with gel-verified full complex (iv, DNA/PNA-peptide-Ab) was present in the chamber above the pore, over 40% of events produced a deeper and longer event signature not observed without the full complex present. These results were replicated in a separate experiment with a 25 nm diameter pore also. In this way, the scaffold/fusion reagent is an key component in our assay for selective detection of target protein analytes. Critically, all other target proteins (like the HIV Ab) either pass through the pore undetected, do not pass through the pore, or produce a fast transient signal that is indistinguishable from other molecules comparable in size/charge that do pass through the pore [Plesa, Calin, Stefan W. Kowalczyk, Ruben Zinsmeester, Alexander Y. Grosberg, Yitzhak Rabin, and Cees Dekker. “Fast translocation of proteins through solid state nanopores.” Nano letters 13, no. 2 (2013): 658-663]. Using the mathematical framework derived previously, the full HIV target-bound complex was detected with 99% confidence in less than 2 minutes (FIG. 54B, lower right). Building on this preliminary success at designing and testing a fusion complex for the HIV biomarkers, we next tested the ability of our method of target analyte detection to work in concert with payload detection.

Detection of 5.6 kb Scaffold with One Payload and One HIV Ab-Bound Site

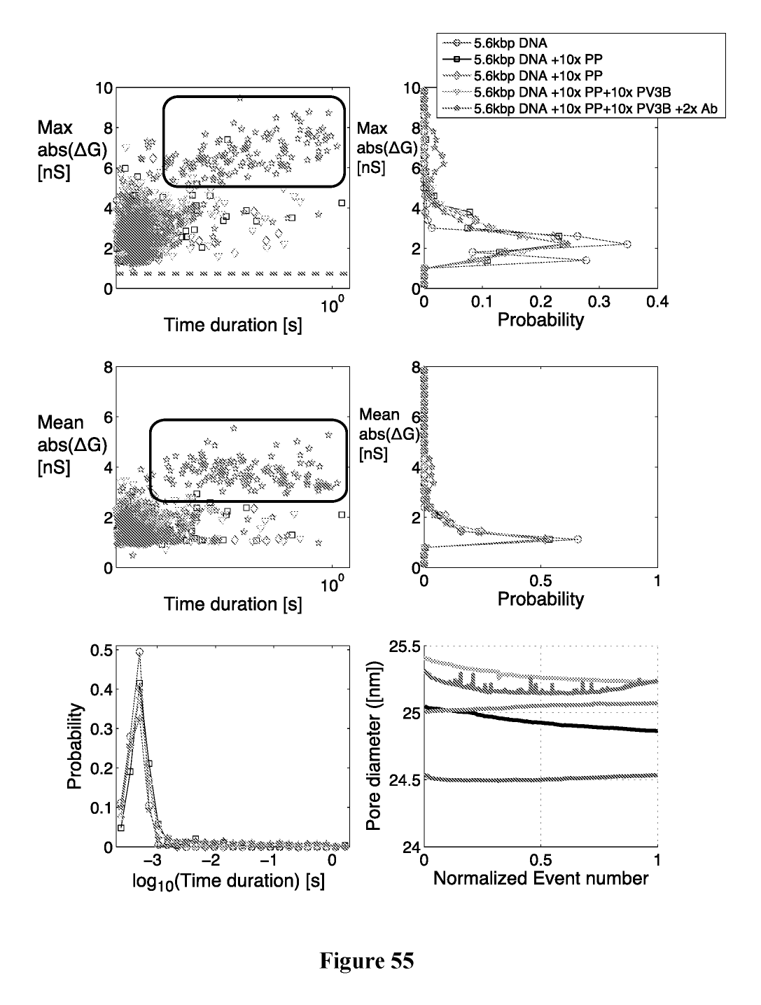

FIG. 55 shows the event plots and histograms for 0.3 nM 5631 bp DNA alone (circles); DNA with 10×PNA-10 kDa PEG (PP) (repeated twice: squares, diamonds); DNA with 10× PP and 10×PNA-V3 loop peptide (PV3B) (triangles); and DNA with 10×PP, 10×PV3B and 2×HIV Ab to binding sites (stars). The 5.6 kb DNA comprised 2 sites per DNA for PNA binding (FIG. 26), and the PP and PV3B used the same PNA binding sequence in this work. Note that unique PNA sequences can be used to increase the likelihood of having only 1 PP and 1 PV3B per scaffold. Since the PNA sequences were the same, the initial 10×PP was meant to bind the majority of scaffolds with only 1 of the PNA binding sites, followed by 10×PV3B meant to occupy the remaining PNA binding site on the majority of scaffolds. The reagents were sequentially tested on the same 24.5-25.5 nm diameter pore.

Between all reagent sets, event-free (i.e., 10 or less events) periods of buffer only were recorded for 5-10 min, to ensure each reagent tested was measured with minimal cross-sample contamination. The 5.6 kb DNA alone at 0.3 nM produced 457 events in 10 minutes, followed by 0.3 nM DNA with 10× (6 nM=2×3 nM per site) PNA-PEG (10 kDa) producing 294 events in 8 minutes, and subsequently a repeat of 0.3 nM DNA with 10×PNA-PEG (10 kDa) producing 316 events in 7 minutes. Next, DNA with 10×PP and 10×V3B produced 787 events in 15 minutes, followed by DNA with 10×PP and 10×V3B and 2×Ab which produced 828 events in 25 minutes. As a final control, 2×Ab alone (0.6 nM) was run, producing only 7 events in 10 minutes.

The DNA alone population is distinguishable from the others, producing the standard folded, partially folded and unfolded event profiles. The DNA with 10×PP and PV3B populations are comparable in event distributions, while the full complex with Ab present (as in the payload free case, FIG. 54) produces a deeper and longer lasting population (FIG. 55, box around events, upper and middle left). These aggregate trends using the standard event plots shows that payload binding is detectable, and target-analyte binding is subsequently detectable also.

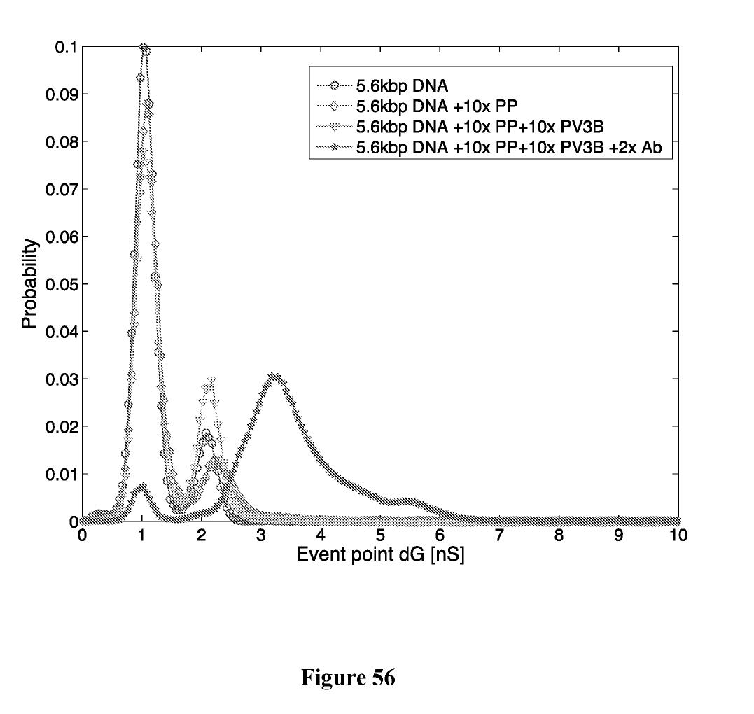

To compare the populations further, FIG. 56 shows an all point histogram for the five sets of reagents. As in Example 9, the DNA alone shows two peaks, 1 nS for unfolded and 2 nS for folded passage. The peaks are shifted to smaller values compared to Example 9 since the pore is larger (25 nm vs. 19 nm). The presence of the PNA-PEG payload creates a shift toward 3 nS, again which is less pronounced that will the smaller pore (FIG. 39). The addition of PNA-peptide does not appear to alter the histogram compared to the payload+DNA population, while the addition of Ab creates a large shift toward deeper events. This is anticipated since Ab-bound to DNA/PNA-peptide complexes produce longer lasting events (FIGS. 54 and 55).

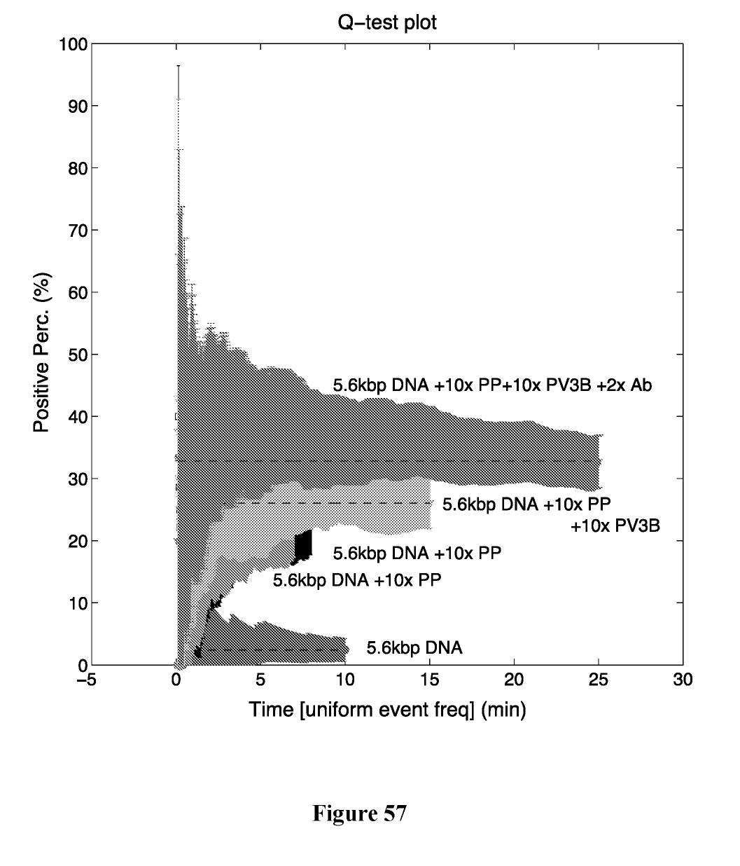

FIG. 57 shows the percentage of events with max abs(ΔG)>3 nS. The fraction and error bars (reporting at 99% confidence) are computed using the method cited in Example 8. The DNA alone produced a final Q=2.407+/−1.8467; DNA with 10×PNA-PEG had Q=24.1497+/−6.4295 and 23.4177+/−6.1363, showing conservation in the event populations when this reagents was twice measured; DNA with 10×PNA-PEG and 10×PNA-peptide had Q=26.0483+/−4.0299, only marginally higher than without the PNA-peptide, suggesting it did not register the way that a second payload would (which is consistent with the peptide being significantly smaller than the 10 kDa PEG payload); and DNA with 10×PNA-PEG, 10×PNA-peptide and 2×Ab had Q=32.7295+/−4.2003. As a result, positive detection of HIV Ab-bound scaffold/fusion is accomplished with 99% confidence since 32.7-4.2=28.5% is larger than the percentages of all the negative controls (the time of first-detection at 99% was 3 minutes, which is the first time the purple lower error bar exceeds 26% in FIG. 57).

### Applying Multi-Level Detection Method

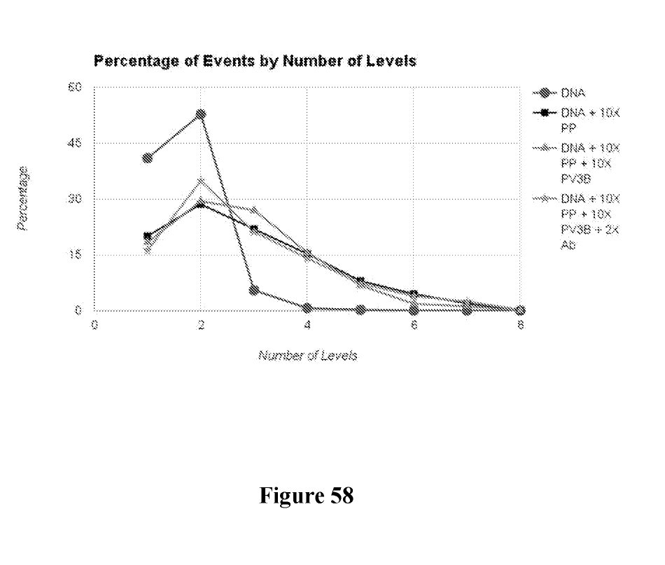

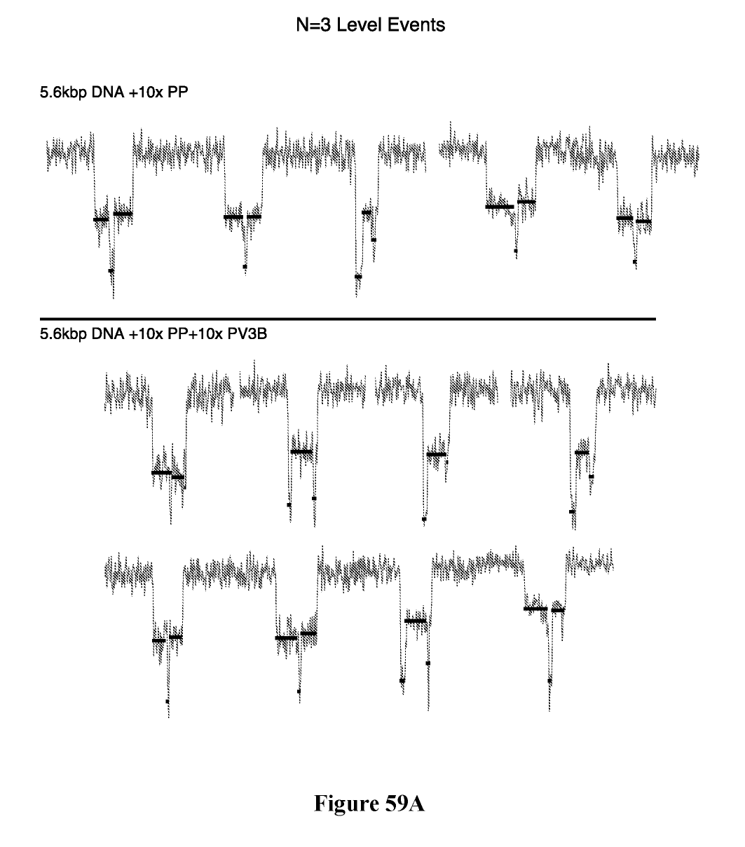

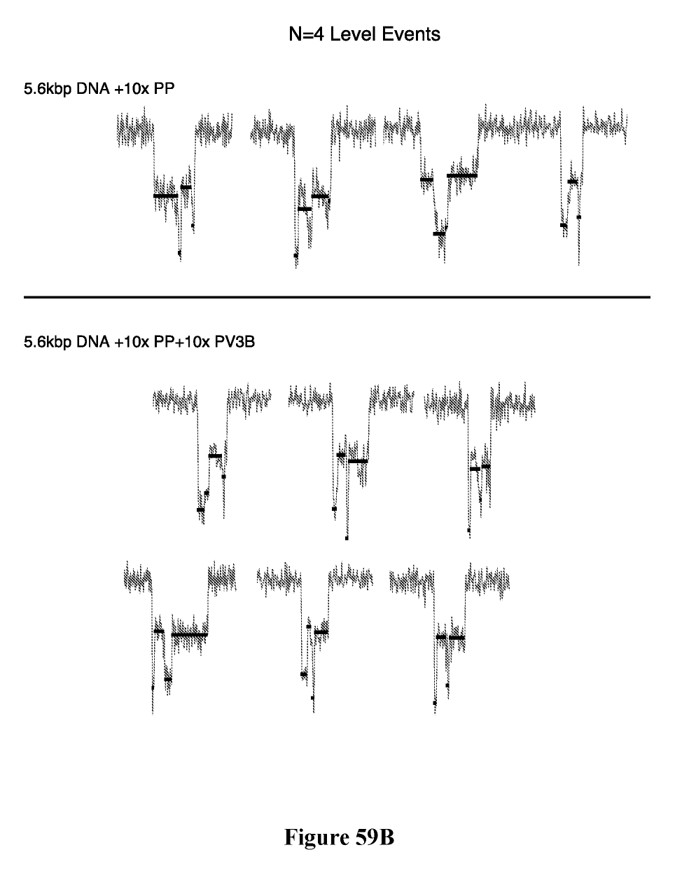

FIG. 58 shows the breakdown (by percentage) of the events by number of identified levels, for DNA alone, DNA with 10×PNA-PEG (second set only), DNA with 10×PNA-PEG and 10×PNA-peptide, and DNA with 10×PNA-PEG and 10×PNA-peptide and 2×Ab. As before in Example 9, the addition of 10×PNA-PEG increases the number of events with 3 or more levels (from 6% for DNA alone to 52% for DNA-PNA-PEG), with the majority of representative events showing a single payload “spike” (N=3 and 4 level examples are in FIGS. 59A and B, respectively).

In adding 10×PNA-peptide to the DNA/PNA-PEG reagent, the trend in FIG. 58 shows that the fraction of events by level is conserved. That is, there is not an visible increase in the number of events with both sites on the DNA occupied, one with a PNA-PEG payload and one with the PNA-peptide. One rationale is that the peptide is too small (1.5 kDa), compared to the 10 kDa PEG, to produce a visible “spike”. This is also clear from the N=3 and N=4 representative events in FIGS. 59A and 59B, where a middle spike or a terminal spike, but not both, tends to occur with both PNA-PEG and PNA-peptide present.

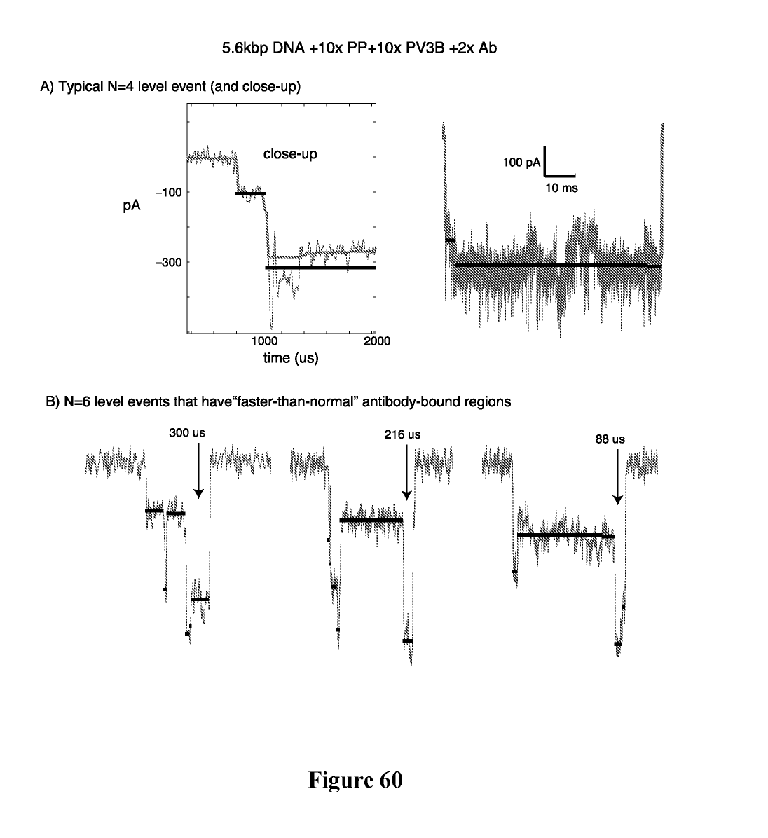

With the addition of antibody, the number of detected levels increases dramatically, and the algorithm uses optimization (interior-point LP for denoising) to smooth the signal before level detection is reapplied. FIG. 60A shows a representative event with N=4 levels detected, the most significant being in a lower amplitude state than previously observed (consistent with the other HIV Ab assays reported). A close-up of the event at the initial transition reveals what may be a payload feature, though it is difficult to tell compared to the other fluctuations within the signal that occur closer to the deepest region of the event amplitude. FIG. 60B shows representative events that are also in the presence of HIV Ab. Interestingly, although the aggregate signals may or may not have been flagged as having Ab bound, the level information reveals a pattern not observed in the other controls, suggesting specifically that the terminal level(s) at the deepest amplitudee are antibody-bound portions of the molecule passing through the pore.

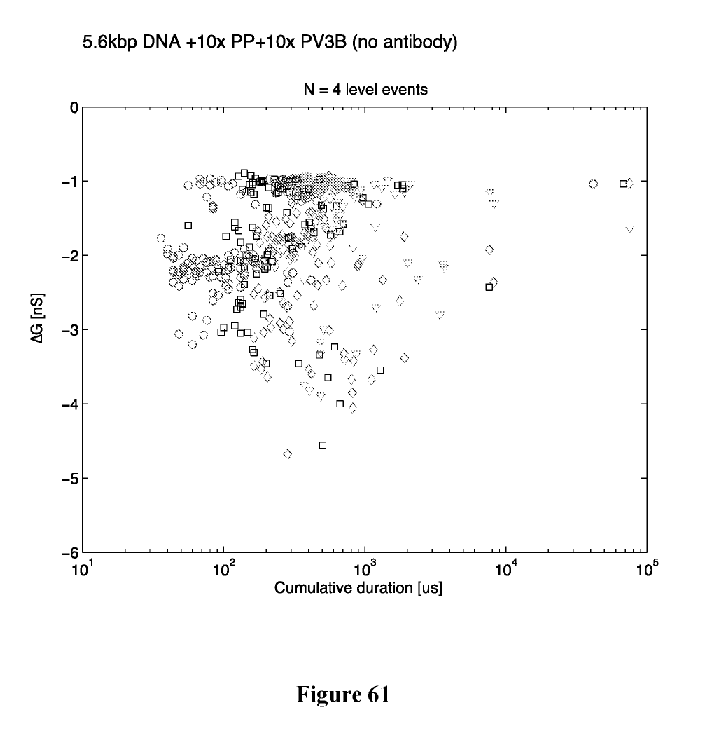

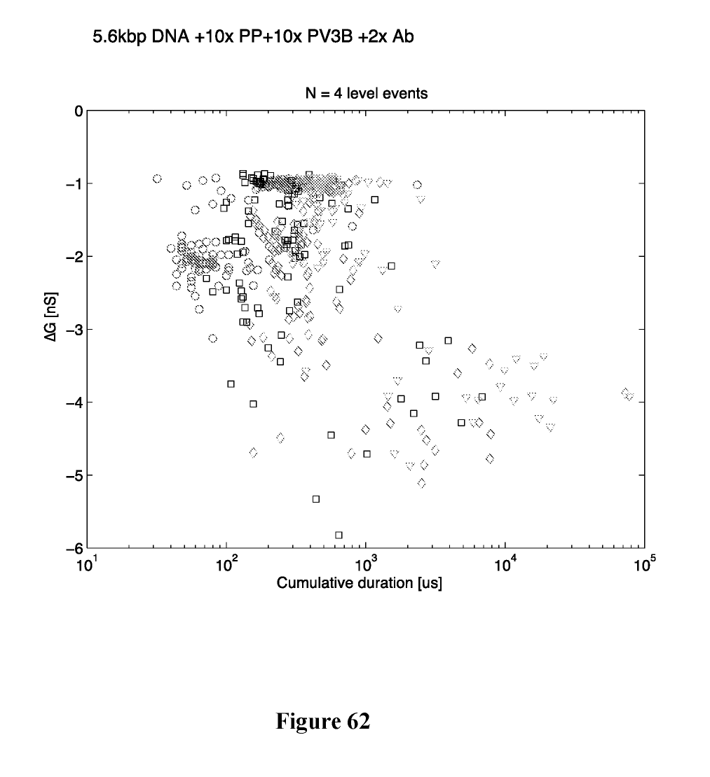

Examining the N=4 level plots for DNA+10×PP+10×PV3B without (FIG. 61) and with (FIG. 62) the HIV antibody shows that, by comparison, the presence of antibody causes one or more of the terminal levels (square=2, diamond=3, triangle=4) to be longer lasting and/or deeper in amplitude. Such detail is not possible when looking only at the aggregate (max or mean absolute value shift vs. duration) information for every event.

Aggregate event information can be used to mathematically model the binding interaction between the scaffold/fusion and the target analyte (e.g., to predict Kd, and predict concentration of analyte when known). An aspect of the novelty of this application is to exploit the multi-level information that can be gleaned from each event, and to leverage that data to advance the modeling goals of each assay, including identifying if more than one binding mode exists, and to compare estimates (e.g., Kd) when using subpopulations of events based on their multi-level characteristics.

Improved detection of “spikes” within events can enable more accurate classification of event types, particularly whether a scaffold has a payload or not, and whether a scaffold has both payload and target bound. In the data set shown here, the target-bound events often swamp the effect of the payload bound within the event signature (FIG. 60A). This has the advantage of have a low false negative for such events. Other assays may produce subtler binding signatures that are comparable to the “spike” that the 10 kDa PEG payload produces, in which case the two payload detection results from Example 9 can be informative. As the multi-level fitting method improves, and tagging of antibody binding consequently becomes more robust, new methods for modeling can be developed.

### Example 11: Conjugation and Purification of a Locked Nucleic Acid (LNA) Molecule to 10 kDa PEG

This Example shows that the scaffold binding domain for a payload (i.e., a label) or for a target analyte binding domain (i.e., a fusion molecule) can be accomplished by using alternatives to PNAs, namely by employing a locked nucleic acid (LNA). A custom made LNA molecule was synthesized and purified by a commercial vendor (Exiqon). The sequence of the LNA that was produced is as follows:

### Disulfide—G GAG C T+G+A+T G G C GTA

where A, G, C, and T are standard DNA bases and + denotes the position of a locked nucleic acid modified base. The 15 residue LNA was conjugated on its 5′ terminus with a disulfide modification for downstream reduction, and PEG conjugation.

In order to conjugate PEG to the LNA, 80 uL of 30 uM LNA was first reduced with 1000-fold molar excess of the reducing agent (tris(2-carboxyethyl)phosphine), otherwise known as TCEP (Thermofisher, Cat #20490). This reduction was carried out in 0.01M sodium phosphate, pH 7.4, for a period of 1 hr at 37° C.

Following reduction of the disulfide modified LNA, reverse phase chromatography was conducted to separate the reduced molecule from excess TCEP and free reduced thiol groups. Samples were ran at 0.8 mL/min using the following protocol, where Buffer A is 50 mM triethylammonium acetate, also known as TEAA (Glen Research), and Buffer B is acetonitrile:

| Time (min) | % Buffer B |
|---|---|
| 0 | 5 |
| 5 | 5 |
| 30 | 90 |
| 31 | 100 |
| 41 | 100 |
| 43 | 5 |
| 53 | 5 |

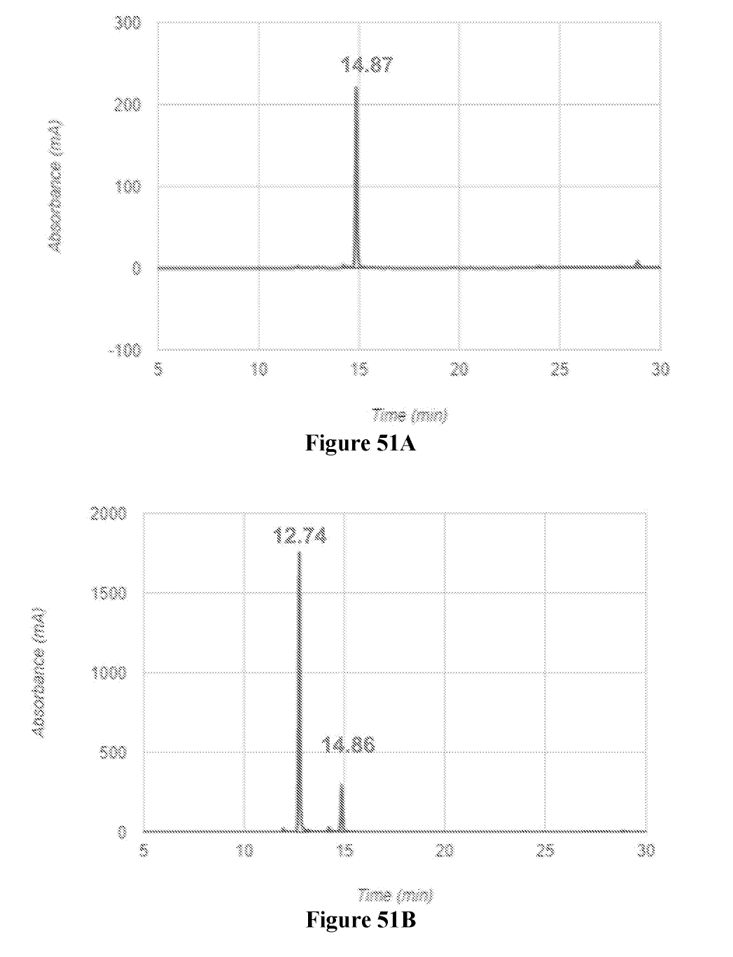

Non-reduced LNA was found to elute at 14.87 min (FIG. 51A) as shown by its absorbance at 260 nm. Following reduction as described above, the peak eluted at 12.74 min (FIG. 51B). This peak representing the reduced LNA molecule was collected in a 1.5 mL centrifuge tube and dried down in a Speed Vac Plus (Savant, Cat #SC110) to rid the sample of HPLC buffers.

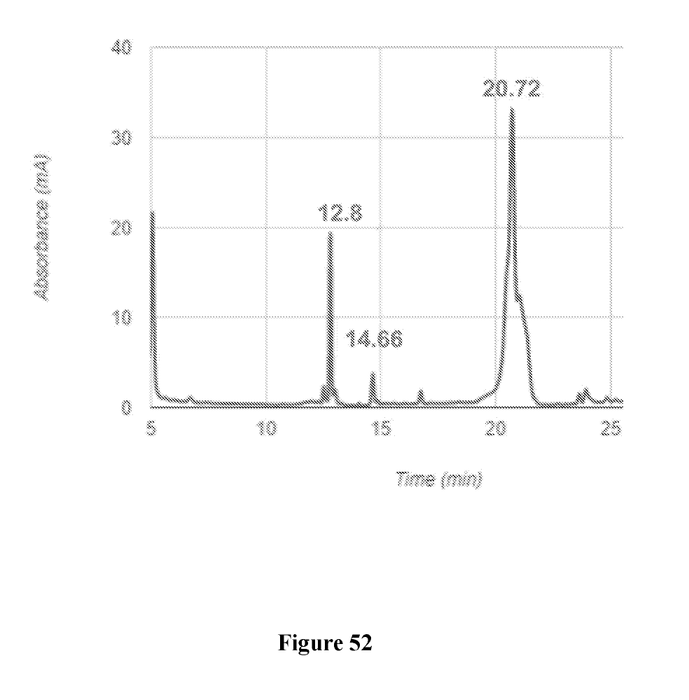

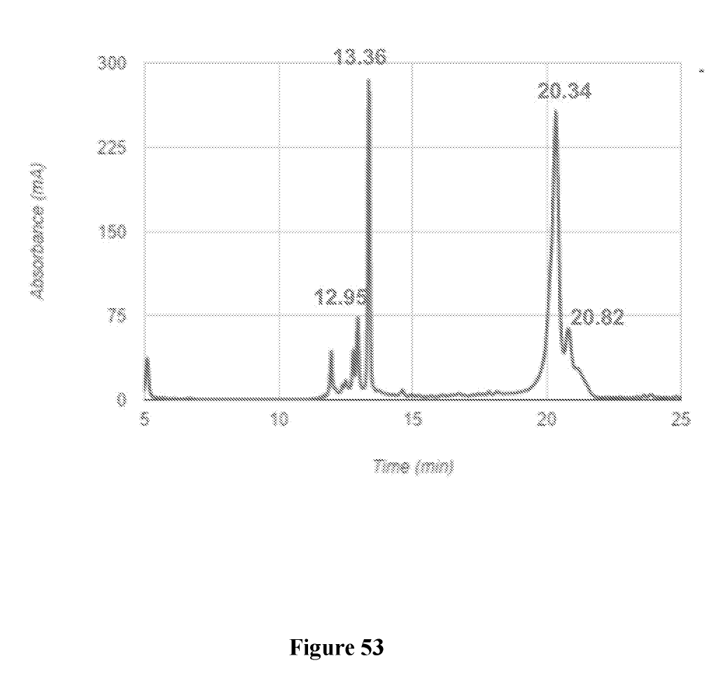

Following collection and dry down of purified, reduced LNA, the sample was resuspended in 80 uL of 0.01 sodium phosphate pH 7.4 for an approximate concentration of 15 uM. This resuspended, purified LNA was then allowed to incubate with a 100-fold molar excess of 10 kDa PEG for a period of 30 minutes at room temperature. After this brief incubation, the conjugation product was analyzed via RP-HPLC using the same protocol as previous. Compared to the main elution peak of 10 kDa PEG at 20.72 min as seen in FIG. 52, the conjugated sample exhibited a new peak at 260 nm that eluted at 20.34 min, indicative of successful LNA conjugation to the 10 kDa PEG molecule (FIG. 53). This peak was again collected and dried down for downstream DNA binding.

### Example 12: Conjugation and Purification of BNA-PEG

HPLC purified bridged nucleic acid (BNA) containing a cysteine (Cys) residue for downstream conjugation was purchased directly from a commercial vendor (Bio-Synthesis, Inc.) of the following sequence:
Cys-PEG₆-T G+G T+A+G T T G+G A G C T G+A+T
   where PEG6 is a 6 unit polyethylene glycol linker, A, G, T and C are standard DNA bases, and (+) denotes the position of a modified nucleic acid analogue known as “bridged”. Bridged nucleic acid 2′,4′-BNANC (2′-0,4′-aminoethylene bridged nucleic acid) is a compound containing a six-member bridged structure with an N—O linkage.

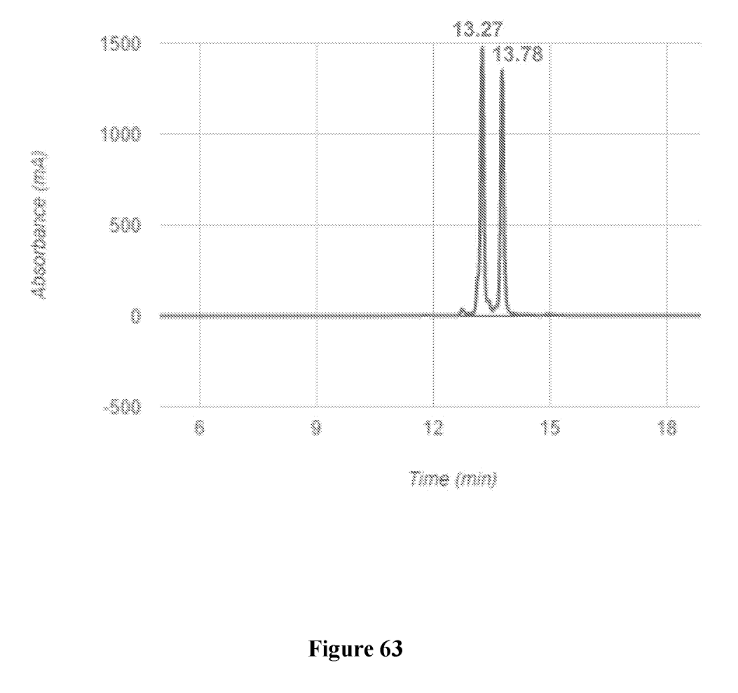

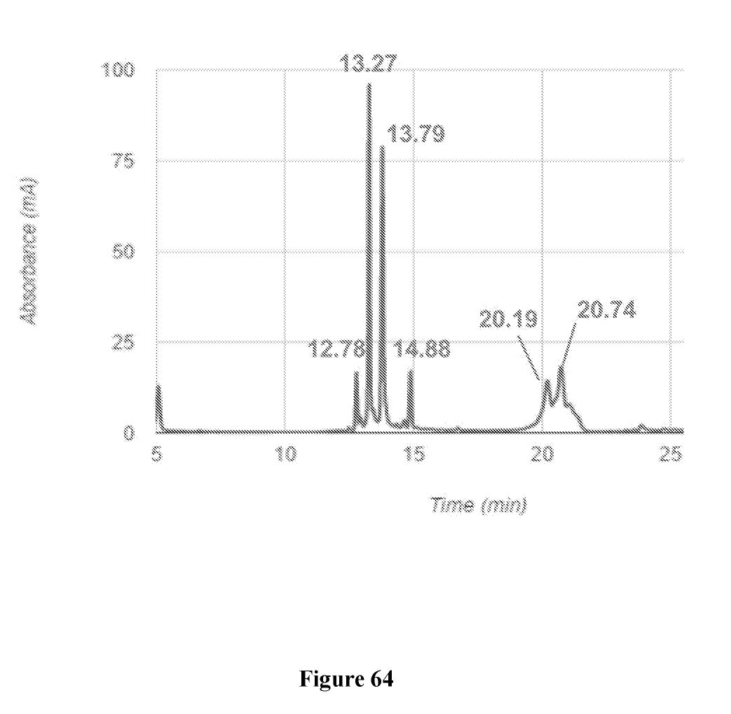

In order to link a 10 kDa maleimide-tagged PEG to the cysteine containing BNA molecule, the two were allowed to incubate together for a period of 30 minutes at room temperature with 100-fold excess PEG (0.01M sodium phosphate, pH 7.4) relative to BNA. Following the brief incubation, the reaction mixture was submitted to HPLC for comparison to the reactants traces alone. Compared to the trace of PEG alone (FIG. 52), and BNA molecule alone (FIG. 63), the reaction product mixture exhibited a new peak at 260 nm at an elution time of 20.19 min, indicative of a PEG-PNA conjugate (FIG. 64). This molecule can then be collected, dried down, and used in DNA binding assays analogous to the LNA-PEG conjugate.

### Example 13: Conjugation and Purification of a Locked Nucleic Acid (LNA) Molecule to an HIV Peptide

The conjugation of the previously described HIV peptide to the LNA molecule proceeded exactly analogous to the LNA-PEG conjugation procedure. The LNA was reduced, purified via HPLC, allowed to react with the HIV peptide, and the reaction product was analyzed and purified by reverse phase chromatography for downstream DNA hybridization.

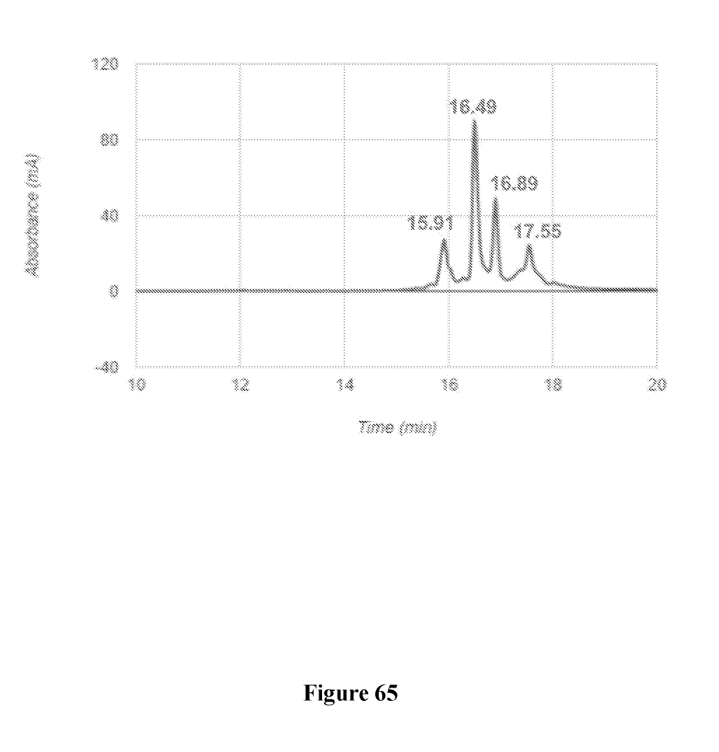

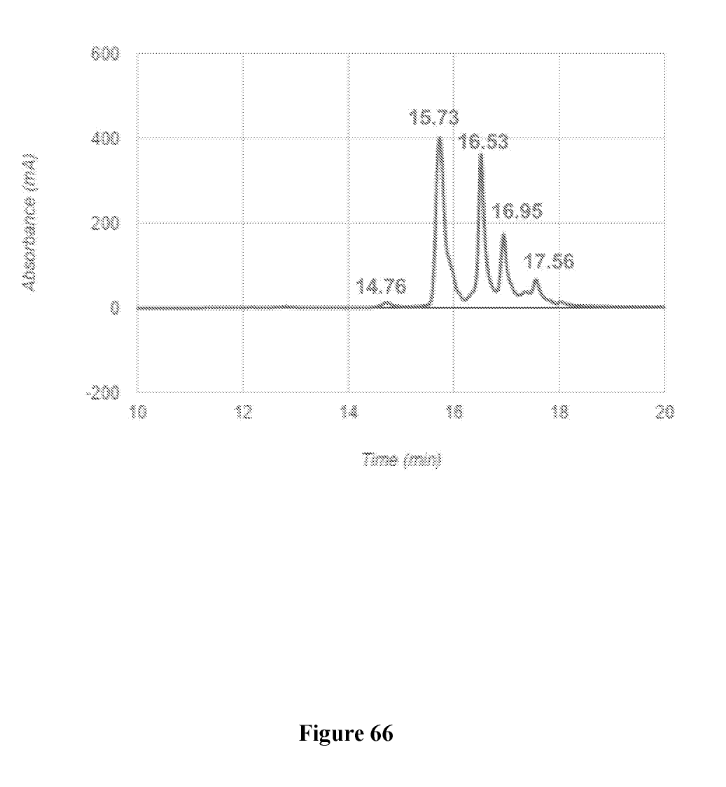

Compared to the HPLC trace of peptide alone (FIG. 65), the reaction mixture produced a new, strong absorbance peak at 15.73 min, indicative of a conjugation product between LNA and the HIV peptide. The peak of LNA alone normally seen at 12.74 min (FIG. 51B) is not present in FIG. 66, indicating complete reaction of all available LNA probe with the HIV peptide.

### Probe Conjugate Hybridization Assay

The LNA portion of these LNA probe conjugates encodes sequence labeling specificity. It was designed to hybridize and form a duplex with a 15-nucleotide sequence located on the reverse strand of the 89 bp dsDNA.

Probe-flanking oligos were added to hybridization reactions. These oligos also bound to the reverse strand of the 89 bp dsDNA; specifically to the remaining nucleotides upstream and downstream from those targeted by the LNA-probe and its conjugates. The function of these flanking oligonucleotides was to both guide the LNA probe to its target, and critically, to prevent the LNA probe from being displaced by the original forward strand of the dsDNA during the reaction cool down.

The LNA-HIV peptide or LNA-10 kDa PEG probe conjugates were then hybridized to an 89 bp fragment of double stranded (ds)DNA containing the LNA probe target sequence which enabled its specific detection by nanopore.

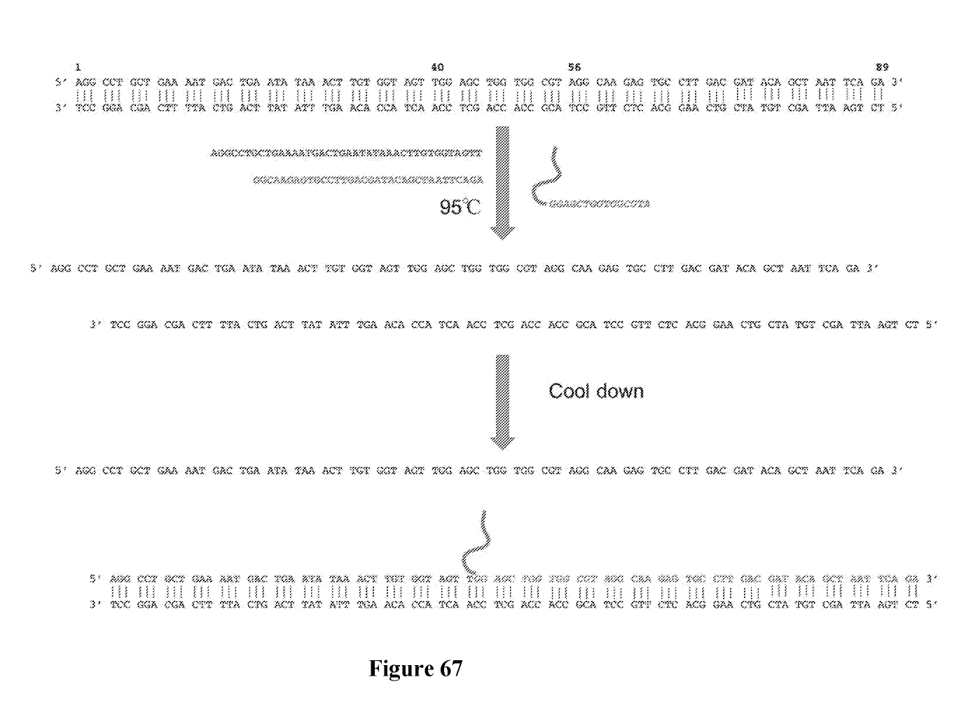

LNA-HIV peptide probe and the probe-flanking oligonucleotides were added at 20-fold excess to the 89 bp dsDNA in sodium phosphate buffer solution. The mixture heated to 95 C for 60 seconds in order to denature the dsDNA into forward and reverse single strands. (FIG. 67) This melting process was completed in the presence of the purified LNA-HIV conjugate (FIG. 67, right of arrow), in addition to two separate competing oligonucleotides flanking the LNA-HIV conjugate on either side (FIG. 67, left of arrow).

Following melting the dsDNA in the presence of the LNA-HIV peptide and flanking oligonucleotides, the mixture was cooled from 95° C. to 60° C. at a rate of 2° C. per second (FIG. 67). This cooling process allowed the LNA-HIV peptide as well as the flanking oligonucleotides to effectively anneal to the reverse strand before the native forward strand due to the 20-fold molar excess.

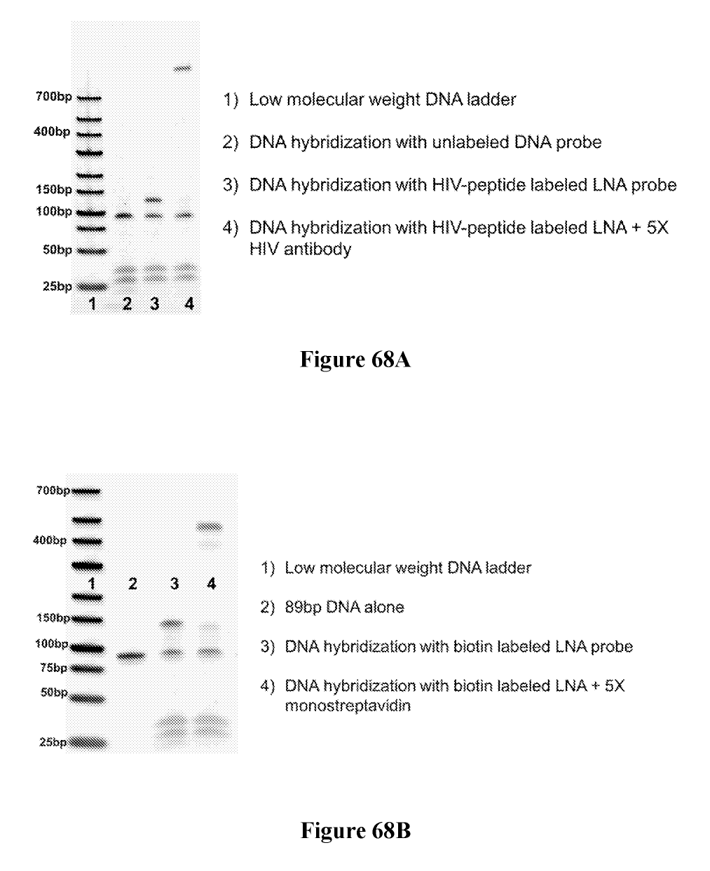

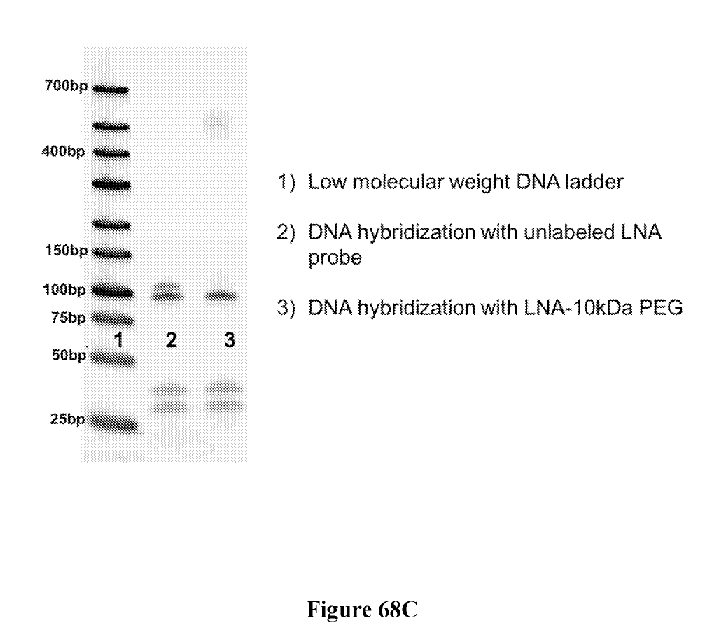

The hybridization products were subsequently ran in a 6% TBE gel (ThermoFisher, Cat # EC62652) for a period of 25 min. at 200V and their electrophoretic mobility was gauged relative to a molecular weight DNA ladder (ThermoFisher, Cat #SM1203). The presence of the HIV peptide on the new dsDNA fragment lowered the electrophoretic mobility of the molecule relative to a hybridization assay completed with a naked DNA probe (FIG. 68A, lanes 3 and 2 respectively). A 5-fold molar excess of HIV antibody (Creative BioLabs, Cat. #DrMAb-136) directed towards the HIV peptide was added to a LNA-HIV peptide hybridized sample and allowed to bind for a period of 5 minutes at room temperature. Successful binding is demonstrated by a significant shift up in the gel of the main band paired with the disappearance of the LNA-peptide hybridization product band (FIG. 68A, lane 4).

This same hybridization approach was also conducted for an LNA probe that was ordered pre-conjugated to a biotin moiety (Exiqon). This biotin group allows downstream conjugation to the protein monostreptavidin (mSA), an analogue of the protein streptavidin (gifted from Mark Howarth, Oxford University). mSA has a single active binding site as opposed to the native four sites of streptavidin, ensuring a 1:1 ratio of LNA-biotin to the labeling protein, while retaining its extremely high affinity towards biotin. In contrast to the native 89 bp dsDNA fragment, hybridization of the LNA-biotin probe and competing nucleotides to the reverse strand resulted in a reduced mobility indicative of a new dsDNA molecule inclusive of the LNA-biotin probe. This resulted in 50% of the DNA being hybridized as described (FIG. 68B, upper band, lane 3) and 50% of the DNA being left as a single stranded forward strand (FIG. 68B, lower band, lane 3). A sample was also prepared that was subsequently labeled with a 5-fold molar excess of mSA. This molecular complex again resulted in an upward shift in the polyacrylamide gel matrix, indicating successful coupling of the new hybridization fragment to the labeling protein (FIG. 68B, lane 4).

Similar to as done previously, the DNA hybridization assay was also completed with an LNA-10 kDa PEG probe that had been purified via HPLC. Successful hybridization by the LNA-10 kDa PEG is observed by an upward shift in an EMSA assay relative to the same assay completed with an unlabeled LNA probe (FIG. 68C).

It is to be understood that while the invention has been described in conjunction with the above embodiments, the foregoing description and examples are intended to illustrate and not limit the scope of the invention. Other aspects, advantages and modifications within the scope of the invention will be apparent to those skilled in the art to which the invention pertains.

1. A method of detecting a target analyte suspected to be present in a mixed sample, comprising:
a. providing a nanopore device comprising a nanopore that separates an interior space of the device into a first volume and a second volume;
b. loading a mixed sample suspected to contain a target analyte into the first volume of said nanopore device;
c. loading a polymer scaffold into the first volume of said nanopore device;
d. configuring the device to pass the polymer scaffold through the nanopore from the first volume to the second volume, wherein said polymer scaffold comprises a label or a detectable tag, and wherein said polymer scaffold comprises a target analyte binding site adapted to bind to said target analyte;
e. recording an electrical signal generated by passage of said polymer scaffold through said nanopore from the first volume to the second volume; and
f. analyzing said electrical signal to determine the presence or absence of a label and the presence or absence of a bound target analyte.

2. The method of claim 1, wherein said analysis of said electrical signal comprises detecting a step transition within an event.

3. The method of claim 2, wherein detecting a step transition event comprises identifying changes in said electrical signal wherein the finite difference exceeds a defined threshold.

4. The method of claim 2, further comprising detecting the presence of at least 2, 3, 4, 5, 6, or 7 levels in an electrical signal.

5. The method of claim 4, further comprising identifying the duration and amplitude of each of said levels.

6. The method of claim 4, further comprising assigning at least one of said levels to a physical status of the polymer scaffold.

7. The method of claim 6, wherein said physical status of the polymer scaffold is selected from the group consisting of: unfolded, folded, label-bound, label-unbound, target analyte-unbound, and target analyte-bound.

8. The method of claim 6, wherein said physical status is assigned using a binning scheme to correlate said level with said physical status.

9. The method of claim 1, wherein said analysis of said electrical signal comprises linear filtering of the electronic signal.

10. The method of claim 1, wherein said analysis of said electrical signal comprises fitting a multi-level approximation to said electrical signal.

11. The method of claim 1, wherein said analysis of said electrical signal distinguishes detection of secondary structure of said polymer scaffold from said label bound to said polymer scaffold.

12. The method of claim 1, wherein said analysis of said electrical signal is computer-implemented.

13. The method of claim 1, wherein said polymer scaffold is bound to a fusion molecule comprising said target analyte binding site.

14. The method of claim 13, wherein said fusion molecule comprises a modified nucleic acid.

15. The method of claim 14, wherein said modified nucleic acid is a conformationally-stabilized nucleic acid.

16. The method of claim 14, wherein said modified nucleic acid is selected from the group consisting of: PNA, LNA, modified DNA, and BNA.

17. The method of claim 13, wherein said fusion molecule comprises an antigen or antibody.

18. The method of claim 13, wherein said polymer scaffold forms a complex comprising said polymer scaffold bound to said fusion molecule bound to said target analyte in the presence of said target analyte, and wherein said complex is adapted to translocate through said nanopore from the first volume to the second volume under an applied voltage.

19. The method of claim 1, wherein said label comprises a modified nucleic acid.

20. The method of claim 19, wherein said modified nucleic acid is a conformationally stabilized nucleic acid.

21. The method of claim 19, wherein said modified nucleic acid is selected from the group consisting of: PNA, LNA, BNA, RNA, and DNA

22. The method of claim 1, wherein said label comprises a molecule selected from the group consisting of: PEG, protein, antibody, DNA, and structured DNA.

23. The method of claim 1, wherein said polymer scaffold comprises dsDNA.

24. The method of claim 1, wherein said polymer scaffold comprises at least one fusion molecule binding domain capable of binding to the fusion molecule.

25. The method of claim 1, wherein said polymer scaffold comprises at least one label binding domain capable of binding to the label.

26. The method of claim 1, wherein said fusion molecule comprises a scaffold binding domain capable of binding to the polymer scaffold at a first target.

27. The method of claim 1, wherein said label comprises a scaffold binding domain capable of binding to the polymer scaffold at a second target

28. The method of claim 1, wherein said fusion molecule provides a unique and detectable electrical signal in a target analyte-bound state as compared to a target analyte-unbound state upon translocation through the nanopore when bound to said polymer scaffold.

29. The method of claim 1, wherein said fusion molecule comprises PNA bound to a molecule comprising a target binding moiety.

30. The method of claim 29, wherein said molecule comprising a target binding moiety comprises an antibody, an aptamer, a affibody, a nanobody, an antibody fragment, an epitope, a hormone, a neurotransmitter, a cytokine, a growth factor, a cell recognition molecule, a nucleic acid, a peptide, a chemical group, chemical modification, or a receptor.

31. The method of claim 1, wherein said target analyte comprises a protein, a peptide, a polynucleotide, a hormone, steroid, intra/extra cellular vesicle, liposome, endosome, nucleated or enucleated cell, mitochondria, virus, viral particle, bacterium, a chemical compound, an ion, or an element.

32. The method of claim 1, wherein said mixed sample comprises an environmental sample or a biological sample.

33. The method of claim 1, wherein said mixed sample comprises whole blood, red blood cells, white blood cells, hair, nails, swabs, urine, sputum, saliva, semen, lymphatic fluid, amniotic fluid, cerebrospinal fluid, peritoneal effusions, pleural effusions, fluid from cysts, synovial fluid, vitreous humor, aqueous humor, bursa fluid, eye washes, eye aspirates, plasma, serum, pulmonary lavage, lung aspirates, liver, spleen, kidney, lung, intestine, brain, heart, muscle, pancreas, primary cell lines, secondary cell lines, or any combination thereof.

34. The method of claim 1, wherein said mixed sample comprises food, water, soil, or waste.

35. The method of claim 1, wherein said device comprises at least two nanopores in series, and wherein said polymer scaffold is simultaneously in said at least two nanopores during translocation.

36. A method of detecting a target analyte suspected to be present in a mixed sample, comprising:
a. loading a polymer scaffold, a fusion molecule, a label, and a mixed sample suspected to contain a target analyte into a device comprising a nanopore that separates an interior space of the device into two volumes, under conditions that allow said label to bind to said polymer scaffold, that allow said fusion molecule to bind to said polymer scaffold, and that allow said fusion molecule to bind to said target analyte,
i. wherein said polymer scaffold is bound to at least one fusion molecule,
ii. wherein said polymer scaffold is bound to at least one label, and
iii. wherein said fusion molecule comprises a target binding domain capable of binding to the target analyte;
b. configuring the device to pass the polymer scaffold in any orientation through the nanopore from one volume to the other volume; and
c. collecting an electrical signal correlated to passage of said polymeric scaffold in any orientation through the nanopore.

37. The method of claim 36, wherein said attachments are covalent.

38. The method of claim 36, wherein said attachments are non-covalent.

39. The method of claim 36, wherein said polymer scaffold comprises at least one fusion molecule binding domain capable of binding to the fusion molecule.

40. The method of claim 36, wherein said polymer scaffold comprises at least one label binding domain capable of binding to the label.

41. The method of claim 36, wherein said fusion molecule comprises a scaffold binding domain capable of binding to the polymer scaffold at a first target.

42. The method of claim 36, wherein said label comprises a scaffold binding domain capable of binding to the polymer scaffold at a second target

43. The method of claim 36, wherein said polymer scaffold comprises dsDNA.

44. The method of claim 36, wherein said polymeric scaffold is dsDNA.

45. The method of claim 36, wherein said fusion molecule provides a unique and detectable electrical signal in a target analyte-bound state as compared to a target analyte-unbound state upon translocation through the nanopore when bound to said polymer scaffold.

46. The method of claim 36, wherein said fusion molecule comprises PNA bound to a molecule comprising a target binding moiety.

47. The method of claim 46, wherein said molecule comprising a target binding moiety comprises an antibody.

48. The method of claim 36, wherein said fusion molecule comprises a polymer scaffold binding domain and an analyte binding domain

49. The method of claim 48, wherein said fusion molecule comprises a zing finger protein, RecA or VspR.

50. The method of claim 36, wherein said mixed sample comprises an environmental sample or a biological sample.

51. The method of claim 36, wherein said mixed sample comprises whole blood, red blood cells, white blood cells, hair, nails, swabs, urine, sputum, saliva, semen, lymphatic fluid, amniotic fluid, cerebrospinal fluid, peritoneal effusions, pleural effusions, fluid from cysts, synovial fluid, vitreous humor, aqueous humor, bursa fluid, eye washes, eye aspirates, plasma, serum, pulmonary lavage, lung aspirates, liver, spleen, kidney, lung, intestine, brain, heart, muscle, pancreas, primary cell lines, secondary cell lines, or any combination thereof.

52. The method of claim 36, wherein said mixed sample comprises food, water, soil, or waste.

53. The method of claim 36, wherein said device comprises at least two nanopores in series, and wherein said polymer scaffold is simultaneously in said at least two nanopores during translocation.

54. A method of analyzing data to detect the presence or absence of a target analyte in a mixed sample, comprising
a. applying a voltage to said mixed sample in a nanopore device comprising a nanopore to translocate molecules in said mixed sample through said nanopore, wherein said mixed sample is suspected of containing a target analyte, wherein said mixed sample comprises background molecules capable of generating an electrical signal in said nanopore, and wherein said mixed sample comprises a polymer scaffold bound to a label and a fusion molecule adapted to bind to said target analyte;
b. obtaining an electrical signal from an event generated by said polymer scaffold translocating through said nanopore;
c. analyzing said electrical signal to detect the presence or absence of a first signature indicating detection of a label bound to the polymer scaffold;
d. analyzing said electrical signal to detect the presence of a second signature indicating detection of a fusion molecule that is bound to said target analyte, or a third signature indicating detection of a fusion molecule that is not bound to said target analyte, wherein the presence of said first and said second signatures in said event indicates the presence of said target analyte in said mixed sample, and wherein the presence of said first and said third signatures indicate the absence of said target analyte in said mixed sample.

55. The method of claim 54, wherein said polymer scaffold comprises dsDNA.

56. The method of claim 54, wherein said polymer scaffold is dsDNA.

57. The method of claim 54, wherein said polymer scaffold comprises at least one fusion molecule binding domain capable of binding to the fusion molecule.

58. The method of claim 54, wherein said polymer scaffold comprises at least one label binding domain capable of binding to the label.

59. The method of claim 54, wherein said fusion molecule comprises a scaffold binding domain capable of binding to the polymer scaffold at a first target.

60. The method of claim 54, wherein said label comprises a scaffold binding domain capable of binding to the polymer scaffold at a second target

61. The method of claim 54, wherein analyzing said event to detect the presence or absence of a first signature comprises comparing said electrical signal to a database comprising a correlation of a signature to a label bound to the polymer scaffold.

62. The method of claim 54, wherein analyzing said event to detect the presence of a second signature comprises comparing said electrical signal to a database comprising a correlation of a signature to a fusion molecule bound to the polymer scaffold that is bound to the target analyte.

63. The method of claim 54, wherein analyzing said event to detect the presence of a third signature comprises comparing said electrical signal to a database comprising a correlation of a signature to a fusion molecule bound to the polymer scaffold that is not bound to the target analyte.

64. The method of claim 54, wherein said mixed sample comprises an environmental sample or a biological sample.

65. The method of claim 54, wherein said mixed sample comprises whole blood, red blood cells, white blood cells, hair, nails, swabs, urine, sputum, saliva, semen, lymphatic fluid, amniotic fluid, cerebrospinal fluid, peritoneal effusions, pleural effusions, fluid from cysts, synovial fluid, vitreous humor, aqueous humor, bursa fluid, eye washes, eye aspirates, plasma, serum, pulmonary lavage, lung aspirates, liver, spleen, kidney, lung, intestine, brain, heart, muscle, pancreas, primary cell lines, secondary cell lines, or any combination thereof.

66. The method of claim 54, wherein said mixed sample comprises food, water, soil, or waste.

67. A kit, comprising (a) a polymer scaffold, (b) a label capable of binding to said polymer scaffold, (c) and a fusion molecule capable of binding to a target ligand and to said polymer scaffold.

68. A method for identifying binding sequences on a polymer scaffold, comprising:
a. providing a polymer scaffold comprising a label binding domain;
b. loading said polymer scaffold and a label adapted to bind to said label binding domain into a device comprising a nanopore that separates an interior space of the device into two volumes, under conditions that allow said label to bind to said label binding sequence;
c. configuring the device to pass the polymer scaffold through the nanopore from one volume to the other volume; and
d. detecting an electrical signal correlated to passage of said polymeric scaffold through the nanopore.

69. The method of claim 68, wherein said polymer scaffold comprises dsDNA.

70. The method of claim 68, wherein said polymer scaffold is dsDNA.

71. The method of claim 68, wherein said electrical signal comprises a measure of current impedance in said nanopore over time.

72. The method of claim 68, wherein said polymer comprises a plurality of label binding sequences, wherein said label binding sequences each bind to a unique label, and wherein each label bound to said nanopore provides a unique electrical signal upon translocation through said nanopore.

73. The method of claim 68, wherein said polymer comprises a plurality of label binding sequences, wherein said label binding sequences each bind to a unique label, and wherein each label provides a unique electrical signature.

74. The method of claim 68, wherein said device comprises at least two nanopores in series, and wherein said polymer scaffold is simultaneously spanning said at least two nanopores during translocation.

75. A compound comprising a polymeric scaffold, a fusion molecule bound to said polymeric scaffold, and a label bound to said polymeric scaffold.

76. The compound of claim 75, wherein said attachment is covalent.

77. The compound of claim 75, wherein said attachment is non-covalent.

78. The compound of claim 75, wherein said polymeric scaffold comprises dsDNA.

79. The compound of claim 75, wherein said polymeric scaffold is dsDNA.

80. The compound of claim 75, wherein said polymeric scaffold comprises a fusion molecule binding domain.

81. The compound of claim 80, wherein said fusion molecule binding domain is bound to a fusion molecule.

82. The compound of claim 75, wherein said polymeric scaffold comprises a label binding domain.

83. The compound of claim 82, wherein said label binding domain is bound to a label.

84. The compound of claim 75, wherein said fusion molecule comprises an antibody, an antibody fragment, an epitope, a hormone, a neurotransmitter, a cytokine, a growth factor, a cell recognition molecule, a nucleic acid, a peptide, an aptamer, a chemical group, a chemical modification, or a receptor, or a receptor.

85. The compound of claim 75, wherein said fusion molecule comprises PNA bound to a molecule comprising a target binding moiety.

86. The compound of claim 85, wherein said molecule comprising a target binding moiety is an antibody or an aptamer.

87. The compound of claim 75, wherein said fusion molecule comprises RecA or VspR.

88. The compound of claim 75, wherein said fusion molecule comprises protein, peptide, PNA, BNA, LNA, CRISPR, TALEN, RNA, or DNA.

89. The compound of claim 88, wherein said DNA is single stranded, double-stranded or multi-stranded.

90. The compound of claim 75, wherein said polymeric scaffold comprises at least two unique fusion molecules bound to said polymeric scaffold.

91. The compound of claim 90, wherein said at least two unique fusion molecules are each bound to a unique target analyte.

92. The compound of claim 75, wherein said fusion molecule is bound to a target analyte.

93. The compound of claim 92, wherein said target analyte comprises a protein, a peptide, a polynucleotide, a chemical compound, an ion, or an element.

94. The compound of claim 93, wherein said target analyte comprises a protein complex or aggregate, a protein/nucleic acid complex, a hormone, a steroid, an intra/extra cellular vessicle, a liposome, an endosome, a nucleated or enucleated cell, a mitochondria, a fragmented or fully assembled virus, a bacterium, a cell, or a cellular aggregate. The compound of claim 75, wherein said fusion molecule is bound to a target analyte via one or more intermediary molecules.

95. The compound of claim 95, wherein said target analyte is further bound to an additional molecule that specifically binds to the target analyte or to the target analyte/fusion molecule complex.

96. The compound of claim 75, wherein said fusion molecule comprises a target binding domain capable of binding to the target analyte, and wherein said fusion molecule comprises a scaffold binding domain capable of binding to the polymer scaffold at a specific target.

97. The compound of claim 96, wherein said specific target comprises a specific polymer sequence.

98. The compound of claim 75, wherein said label comprises PNA.

99. The compound of claim 75, wherein said label comprises a detectable tag.

100. The compound of claim 99, wherein said detectable tag comprises PEG.

101. The compound of claim 75, wherein said label comprises an oligonucleotide, a PNA, a polypeptide, a protein, or an aptamer.

102. The compound of claim 75, wherein said label comprises a scaffold binding domain capable of binding to the polymer scaffold at a specific target.

103. The compound of claim 102, wherein said specific target comprises a specific polymer sequence.

104. The compound of claim 75, wherein said polymeric scaffold comprises a DNA molecule, a PNA molecule, an RNA molecule, or a polypeptide molecule.

105. The compound of claim 75, wherein said polymer scaffold is bound to a plurality of labels, wherein at least two labels have a unique size, shape, hydrophobicity or charge that renders each capable of generating detectably distinct electrical signals in a nanopore device.

106. The compound of claim 75, wherein said polymer scaffold is bound to a plurality of labels, wherein at least two labels are bound consecutively along said polymer scaffold to facilitate generation of a unique and detectable electrical signal in a nanopore device generated by the size, shape, hydrophobicity, or charge of the label as it translocates through a nanopore in a nanopore device.

107. A kit comprising (a) two or more labels each having different size, charge and/or shape and comprising a polymeric scaffold and (b) a nanopore device comprising a nanopore that separates and connects two volumes in the nanopore device, wherein the nanopore device is configured to identify each of the labels when the label is bound to said polymeric scaffold and said polymeric scaffold translocates through said nanopore.


SEQUENCE LISTING (9 sequences)

SEQ ID NO: 1 - protein, 6 aa, Artificial Sequence
  Description of Artificial Sequence Synthetic 6xHis tag
HHHHHH

SEQ ID NO: 2 - protein, 14 aa, Artificial Sequence
  Description of Artificial Sequence Synthetic peptide
  modified_base (1)..(1): modified with Mpa-Ttds
KSIHIGPGRAFYTT

SEQ ID NO: 3 - DNA, 12 bp, Artificial Sequence
  Description of Artificial Sequence Synthetic oligonucleotide
cctttcccttcc

SEQ ID NO: 4 - DNA, 15 bp, Artificial Sequence
  Description of Artificial Sequence Synthetic oligonucleotide
  modified_base (6)..(8): Locked nucleic acid
ggagctgatggcgta

SEQ ID NO: 5 - DNA, 17 bp, Artificial Sequence
  Description of Artificial Sequence Synthetic oligonucleotide
  modified_base (1)..(1): modified with PEG6-Cys
  modified_base (2)..(2): 2′-O,4′-aminoethylene bridged nucleic acid
  modified_base (4)..(5): 2′-O,4′-aminoethylene bridged nucleic acid
  modified_base (9)..(9): 2′-O,4′-aminoethylene bridged nucleic acid
  modified_base (15)..(16): 2′-O,4′-aminoethylene bridged nucleic acid
tggtagttggagctgat

SEQ ID NO: 6 - DNA, 89 bp, Artificial Sequence
  Description of Artificial Sequence Synthetic oligonucleotide
aggcctgctgaaaatgactgaatataaacttgtggtagttggagctggtggcgtaggcaagagtgccttgacgatacagctaattcaga

SEQ ID NO: 7 - DNA, 40 bp, Artificial Sequence
  Description of Artificial Sequence Synthetic oligonucleotide
aggcctgctgaaaatgactgaatataaacttgtggtagtt

SEQ ID NO: 8 - DNA, 34 bp, Artificial Sequence
  Description of Artificial Sequence Synthetic oligonucleotide
ggcaagagtgccttgacgatacagctaattcaga

SEQ ID NO: 9 - DNA, 15 bp, Artificial Sequence
  Description of Artificial Sequence Synthetic oligonucleotide
ggagctggtggcgta